SEQUENCE LISTING (17 sequences)

SEQ ID NO: 1 - DNA, 1232 bp, Homo sapiens
  CDS (19)..(1044)
tttcccgagatcaccaagatgctgggcgccgagtggagcaagctgcagccaacggaaaagcagcggtacctggatgaggccgagagagagaagcagcagtacatgaaggagctgcgggcgtaccagcagtctgaagcctataagatgtgcacggagaagatccaggagaagaagatcaagaaagaagactcgagctctgggctcatgaacactctcctgaatggacacaagggtggggactgcgatggcttctccaccttcgatgttcccatcttcactgaagagttcttggaccaaaacaaagcgcgtgaggcggagcttcggcgcttgcggaagatgaatgtggccttcgaggagcagaacgcggtactgcagaggcaaaacgcagagcatgagcagcgcgcgcgagcgtctggagcaggagctggcgctggaggagcggaggacgctggcgctgcagcagcagctccaggccgtgcgccaggcgctcaccgccagcttcgcctcactgccggtgccgggcacgggcgaaacgcccacgctgggcactctggacttctacatggcccggcttcacggagccatcgagcgcgaccccgcccagcacgagaagctcatcgtccgcatcaaggaaatcctggcccaggtcgccagcgagcacctgtgaggagtgggcgggcccacgatgcagaggagaagctgtgggcgcggccctgccacaccccaccccgtggacgagaggctgggggtccaccctttggggcctggtcccatcctgcacctttgggggctccagcccccctaaaattaaatttctgcagcatccctttagctttcaatctccccagccccctgaacccggaaaaagcactcgctgcgcgatacacccagaagaacctcacagccgagggtgcccctcctcggaggacagccacgcgctacactggctctccgggccacccccaggacacagggcagacgaaacccacccccagcacacggcaggaccccccaaattactcactacggggggctgtgccataggccacacaggaagctgccttgtggggacttacctggggtgtcccccgcatgcctgtaccccagatgggtgggggccggctttgcccatcctgctctcctccagccgagggaccctggtgggggtggctccttctcactgctggatccggactttttaaataaaaacaagtaaaatttgtgttttaaaa

SEQ ID NO: 2 - protein, 341 aa, Homo sapiens
MLGAEWSKLQPTEKQRYLDEAEREKQQYMKELRAYQQSEAYKMCTEKIQEKKIKKEDSSSGLMNTLLNGHKGGDCDGFSTFDVPIFTEEFLDQNKAREAELRRLRKMNVAFEEQNAVLQRQNAEHEQRARASGAGAGAGGAEDAGAAAAAPGRAPGAHRQLRLTAGAGHGRNAHAGHSGLLHGPASRSHRARPRPAREAHRPHQGNPGPGRQRAPVRSGRAHDAEEKLWARPCHTPPRGREAGGPPFGAWSHPAPLGAPAPLKLNFCSIPLAFNLPSPLNPEKALAARYTQKNLTAEGAPPRRTATRYTGSPGHPQDTGQTKPTPSTRQDPPNYSLRGAVP

SEQ ID NO: 3 - DNA, 3870 bp, Homo sapiens
  CDS (179)..(1642)
cagggagacatacaggatttaagaagcccatcatggagaagaccttcaattacagagataaaaagtttttcttgtggaacaagttaacactagatggcagataacagactgaggagtgagctgcttctgactcgattaaaaagggagtgagccataactggcggctgctctttcgccaatgagcctccccaattcctcctgcctcttagaagacaagatgtgtgagggcaacaagaccactatggccagcccccagctgatgcccctggtggtggtcctgagcactatctgcttggtcacagtagggctcaacctgctggtgctgtatgccgtacggagtgagcggaagctccacactgtggggaacctgtacatcgtcagcctctcggtggcggacttgatcgtgggtgccgtcgtcatgcctatgaacatcctctacctgctcatgtccaagtggtcactgggccgtcctctctgcctcttttggctttccatggactatgtggccagcacagcgtccattttcagtgtcttcatcctgtgcattgatcgctaccgctctgtccagcagcccctcaggtaccttaagtatcgtaccaagacccgagcctcggccaccattctgggggcctggtttctctcttttctgtgggttattcccattctaggctggaatcacttcatgcagcagacctcggtgcgccgagaggacaagtgtgagacagacttctatgatgtcacctggttcaaggtcatgactgccatcatcaacttctacctgcccaccttgctcatgctctggttctatgccaagatctacaaggccgtacgacaacactgccagcaccgggagctcatcaataggtccctcccttccttctcagaaattaagctgaggccagagaaccccaagggggatgccaagaaaccagggaaggagtctccctgggaggttctgaaaaggaagccaaaagatgctggtggtggatctgtcttgaagtcaccatcccaaacccccaaggagatgaaatccccagttgtcttcagccaagaggatgatagagaagtagacaaactctactgctttccacttgatattgtgcacatgcaggctgcggcagaggggagtagcagggactatgtagccgtcaaccggagccatggccagctcaagacagatgagcagggcctgaacacacatggggccagcgagatatcagaggatcagatgttaggtgatagccaatccttctctcgaacggactcagataccaccacagagacagcaccaggcaaaggcaaattgaggagtgggtctaacacaggcctggattacatcaagtttacttggaagaggctccgctcgcattcaagacagtatgtatctgggttgcacatgaaccgcgaaaggaaggccgccaaacagttgggttttatcatggcagccttcatcctctgctggatcccttatttcatcttcttcatggtcattgccttctgcaagaactgttgcaatgaacatttgcacatgttcaccatctggctgggctacatcaactccacactgaaccccctcatctaccccttgtgcaatgagaacttcaagaagacattcaagagaattctgcatattcgctcctaagggaggctctgaggggatgcaacaaaatgatccttatgatgtccaacaaggaaatagaggacgaaggcctgtgtgttgccaggcaggcacctgggctttctggaatccaaaccacagtcttaggggcttggtagtttggaaagttcttaggcaccatagaagaacagcagatggcggtgatcagcagagagattgaactttgaggaggaagcagaatctttgcaagaaagtcagacctgtttcttgtaactgggttcaaaaagaaaaaaataataaaaataaaagagagagagaatcagacctgggtggaactctcctgctcctcaggaactatgggagcctcagactcattgtaattcaagctttccgagtcaagtgattgacaactgaagagacacgtggctagggttccactggagaattgaaaaggactcttgagccctcctggaatggagctgtataactgtgcagagactttatccatgccaatagttgctgtccccttccaggggtcaccttgagaggcatgacagctgttccacaggggctatcccttctcagaaaacttctcttctgagcctctttaacagctttctccagaaccagtgtctgaaccaccctggaaattctgccttattatttcttactcaaacatgtttagagtggatagaaaattatgcagcttgcacacccatcatctttaaccccaaatttcctttggctattaaaaaagtggtggcaaaaggcatcctcaaaagaaagagaaatgaaatatttttgaatggttgcacgttaaaaattaaaagaaggaatgggggcagaatgccatatttttgagggctgtactaggtttatctcatttaagccccacaacaccccacaggagggtaattttctaactctagtttgcagaggagcaaattgaggttcagcaaggtgagagaggtacccaaggtcacatagctagttatgtgagaaagttagagtacagatcctctggggtttcagcttattgtagcatattttctccgaaaggcaaaaatgtgcccttttggccgggcatggtagctcaagcctataatcccagcatgttgagaggctgaggtgggcagatcatttgaggccaggagttcaagaccagtctggccaatatggagaaaccttgtctctactaaaaacacaaaaattatctgggcatggtggggcatgcctgtagtcccacttacttgggaggccgaggcacgagaatcgcttgaacccgggaggtggaggttgccgtgagccaagatcacgccactgcactccagcctgggcaacagagcaagactctgtctcaaaaaaaaaaatacaatattttaacaatgtgccctcttaagtgtgcacagatacacatacacggtattcccaagagtggtggcagctcaaaatgatatgtttgagtagacgaacagctgacatggagttcccgtgcacctacggaaggggacgctttgaaggaaccaagtgcatttttatctgtgagttctgttgtgtttgtcaaaaagtcattgtaatctttcatagccatacctggtaagcaaaaactagtaaagacataggaacatgtagttttacttggtgtttatgttgcaatctggttgtgatttatattttaaagcttggtgctaaaccacaatatgtatagcacatggagtgcctgtacaagctgatgttttgtattttgtgttcctctttgcatgatctgtcaaagtgagatatttttacctgcctaaaatatgatgtttaaaagcatactctatgtgatttatttatttctacctttctgagtctcttggactaagaagatgttttgaaatgtaccatcaaatgttaacagagtttgatatgggctttctctttggtttctcatcacatttgtaaatgtcttttcaaaaggatttactttttgtaaaaagcttcattctcactctgctttgcatcccccaaacttcttgttcaaaacggggggagtttaggagactttaatcccggtttcagaagctgcagctggtctgtttccaggtcagaaaccattgttcagaagacctccctgtgagagagttgctcctcagggtccctcaggaccaaagaacactcgaaaagagcacttcacacagacaagtggctaagtgtccattatttaccttgaacaatcaaggcaactagtggagagaactgattgtgagctc

SEQ ID NO: 4 - protein, 487 aa, Homo sapiens
MSLPNSSCLLEDKMCEGNKTTMASPQLMPLVVVLSTICLVTVGLNLLVLYAVRSERKLHTVGNLYIVSLSVADLIVGAVVMPMNILYLLMSKWSLGRPLCLFWLSMDYVASTASIFSVFILCIDRYRSVQQPLRYLKYRTKTRASATILGAWFLSFLWVIPILGWNHFMQQTSVRREDKCETDFYDVTWFKVMTAIINFYLPTLLMLWFYAKIYKAVRQHCQHRELINRSLPSFSEIKLRPENPKGDAKKPGKESPWEVLKRKPKDAGGGSVLKSPSQTPKEMKSPVVFSQEDDREVDKLYCFPLDIVHMQAAAEGSSRDYVAVNRSHGQLKTDEQGLNTHGASEISEDQMLGDSQSFSRTDSDTTTETAPGKGKLRSGSNTGLDYIKFTWKRLRSHSRQYVSGLHMNRERKAAKQLGFIMAAFILCWIPYFIFFMVIAFCKNCCNEHLHMFTIWLGYINSTLNPLIYPLCNENFKKTFKRILHIRS

SEQ ID NO: 5 - DNA, 3465 bp, Homo sapiens
agtattggagtgttacagggagacatacaggatttaagaagcccatcatggagaagaccttcaattacagagataaaaagtttttcttgtgaacaagttacactagatggaagataacagactgaggagtgagctgcttctgactcgattaaaaagggagtgagccataactggcggctgctcttgcgccaatgagcctccccaattcctcctgcctcttagaagacaagatgtgtgagggcaacaagaccactatggccagcccccagctgatgcccctggtggtggtcctgagcactatctgcttggtcacagtagggctcaacctgctggtgctgtatgccgtacggagtgagcggaagctccacactgtggggaacctgtacatcgtcagcctctcggtggcggacttgatcgtgggtgccgtcgtcatgcctatgaacatcctctacctgctcatgtccaagtggtcactgggccgtcctctctgcctcttttggctttccatggactatgtggccagcacagcgtccattttcagtgtcttcatcctgtgcattgatcgctaccgctctgtccagcagcccctcaggtaccttaagtatcgtaccaagacccgagcctcggccaccattctgggggcctggtttctctcttttctgtgggttattcccattctaggctggaatcacttcatgcagcagacctcggtgcgccgagaggacaagtgtgagacagacttctatgatgtcacctggttcaaggtcatgactgccatcatcaacttctacctgcccaccttgctcatgctctggttctatgccaagatctacaaggccgtacgacaacactgccagcaccgggagctcatcaataggtccctcccttccttctcagaaattaagctgaggccagagaaccccaagggggatgccaagaaaccagggaaggagtctccctgggaggttctgaaaaggaagccaaaagatgctggtggtggatctgtcttgaagtcaccatcccaaacccccaaggagatgaaatccccagttgtcttcagccaagaggatgatagagaagtagacaaactctactgctttccacttgatattgagcacatgcaggctgcggcagaggggagtagcagggactatgtagccgtcaaccggagccatggccagctcaagacagatgagcagggcctgaacacacatggggccagcgagatatcagaggatcagatgttaggtgatagccaatccttctctcgaacggactcagataccaccacagagacagcaccaggcaaaggcaaattgaggagtgggtctaacacaggcctggattacatcaagtttacttggaagaggctccgctcgcattcaagacagtatgtatctgggttgcacatgaaccgcgaaaggaaggccgccaaacagttgggttttatcatggcagccttcatcctctgctggatcccttatttcatcttcttcatggtcattgccttctgcaagaactgttgcaatgaacatttgcacatgttcaccatctggctgggctacatcaactccacactgaaccccctcatctaccccttgtgcaatgagaacttcaagaagacattcaagagaattctgcatattcgctcctaagggaggctctgaggggatgcaacaaaatgatccttatgatgtccaacaaggaaatagaggacgaaggcctgtgtgttgccaggcaggcacctgggctttctggaatccaaaccacagtcttaggggcttggtagtttggaaagttcttaggcaccatagaagaacagcagatggcggtgatcagcagagagattgaactttgaggaggaagcagaatctttgcaagaaagtcagacctgtttcttgtaactgggttcaaaaagaaaaaaataataaaaataaaagagagagagaatcagacctgggtggaactctcctgctcctcaggaactatgggagcctcagactcattgtaattcaagctttccgagtcaagtgattgacaactgaagagacacgtggctagggttccactggagaattgaaaaggactcttgagccctcctggaatggagctgtataactgtgcagagactttatccatgccaatagttgctgtccccttccaggggtcaccttgagaggcatgacagctgttccacaggggctatcccttctcagaaaacttctcttctgagcctctttaacagctttctccagaaccagtgtctgaaccaccctggaaattctgccttattatttcttactcaaacatgtttagagtggatagaaaattatgcagcttgcacacccatcgtctttaaccccaaatttcctttggctattaaaaaagtggtggcaaaaggcatcctcaaaagaaagagaaatgaaatatttttgaatggttgcacgttaaaaattaaaagaaggaatgggggcagaatgccatatttttgagggctgtactaggtttatctcatttaagccccacaacaccccacaggagggtaattttctaactctagtttgcagaggagcaaattgaggttcagcaaggtgagagaggtacccaaggtcacatagctagttatgtgagaaagttagagtacagatcctctggggttttcagcttattgtagcatattttctccgaaaggcaaaaatgtgcccttttggccgggcatggtagctcaagcctataatcccagcatgttgagaggctgaggtgggcagatcatttgaggccaggagttcaagaccagtctggccaatatggagaaaccttgtctctactaaaaacacaaaaattatctgggcatggtggggcatgcctgtagtcccacttacttgggaggccgaggcacgagaatcgcttgaacccgggaggtggaggttgccgtgagccaagatcacgccactgcactccagcctgggcaacagagcaagactctgtctcaaaaaaaaaaaatacaatattttaacaatgtgccctcttaagtgtgcacagatacacatacacggtattcccaagagtggtggcagctcaaaatgatatgtttgagtagacgaacagccgacatggagttcccgtgcacctacggaaggggacgctttgaaggaaccaagtgcatttttatctgtgagttctgttgtgtttgtcaaaaagtcattgtaatctttcatagccatacctggtaagcaaaaactagtaaagacataggaacatgcagttttacttggtgtttatgttgcaatctggttgtgatttatattttaaagcttggtgctaaaccacaatatgtatagcacatggagtgcctgtacaagctgatgttttgtattttgtgttcctctttgcatgatctgtcaaagtgagatatttttacctgcctaaaatatgatgtttaaaagcataaaaaaaaaaaaaaaa

SEQ ID NO: 6 - DNA, 1418 bp, Homo sapiens
  CDS (110)..(979)
aactgtgcgaaccagacccggcagccttgctcagttcagcatagcggagcggatccgatcggatcggagcacaccggagcaggctcatcgagaaggcgtctgcgagaccatggagaacggatacacctatgaagattataagaacactgcagaatggcttctgtctcatactaagcaccgacctcaagttgcaataatctgtggttctggattaggaggtctgactgataaattaactcaggcccagatctttgactacagtgaaatccccaactttcctcgaagtacagtgccaggtcatgctggccgactggtgtttgggttcctgaatggcagggcctgtgtgatgatgcagggcaggttccacatgtatgaagggtacccactctggaaggtgacattcccagtgagggttttccaccttctgggtgtggacaccctggtagtcaccaatgcagcaggagggctgaaccccaagtttgaggttggagatatcatgctgatccgtgaccatatcaacctacctggtttcagtggtcagaaccctctcagagggcccaatgatgaaaggtttggagatcgtttccctgccatgtctgatgcctacgaccggactatgaggcagagggctctcagtacctggaaacaaatgggggagcaacgtgagctacaggaaggcacctatgtgatggtggcaggccccagctttgagactgtggcagaatgtcgtgtgctgcagaagctgggagcagacgctgttggcatgagtacagtaccagaagttatcgttgcacggcactgtggacttcgagtctttggcttctcactcatcactaacaaggtcatcatggattatgaaagcctggagaaggccaaccatgaagaagtcttagcagctggcaaacaagctgcacagaaattggaacagtttgtctccattcttatggccagcattccactccctgacaaagccagttgacctgccttggagtcgtctggcatctcccacacaagacccaagtagctgctaccttctttggccccttgctggagtcatgtgcctctgtccttaggttgtagcagaaaggaaaagattcctgtccttcacctttcccactttcttctaccagacccttctggtgccagatcctcttctcaaagctgggattacaggtgtgagcatagtgagaccttggcgctacaaaataaagctgttctcattcctgttctttcttacacaagagctggagcccgtgccctaccacacatctgtggagatgcccaggatttgactcgggccttagaactttgcatagcagctgctactagctctttgagataatacattccgaggggctcagttctgccttatctaaatcaccagagaccaaacaaggactaatccaatacctcttgga

SEQ ID NO: 7 - protein, 289 aa, Homo sapiens
MENGYTYEDYKNTAEWLLSHTKHRPQVAIICGSGLGGLTDKLTQAQIFDYSEIPNFPRSTVPGHAGRLVFGFLNGRACVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAECRVLQKLGADAVGMSTVPEVIVARHCGLRVFGFSLITNKVIMDYESLEKANHEEVLAAGKQAAQKLEQFVSILMASIPLPDKAS

SEQ ID NO: 8 - DNA, 4343 bp, Homo sapiens
  CDS (11)..(1642)
agatttgataatgggctgcattaaaagtaaagaaaacaaaagtccagccattaaatacagacctgaaaatactccagagcctgtcagtacaagtgtgagccattatggagcagaacccactacagtgtcaccatgtccgtcatcttcagcaaagggaacagcagttaatttcagcagtctttccatgacaccatttggaggatcctcaggggtaacgccttttggaggtgcatcttcctcattttcagtggtgccaagttcatatcctgctggtttaacaggtggtgttactatatttgtggccttatatgattatgaagctagaactacagaagacctttcatttaagaagggtgaaagatttcaaataattaacaatacggaaggagattggtgggaagcaagatcaatcgctacaggaaagaatggttatatcccgagcaattatgtagcgcctgcagattccattcaggcagaagaatggtattttggcaaaatggggagaaaagatgctgaaagattacttttgaatcctggaaatcaacgaggtattttcttagtaagagagagtgaaacaactaaaggtgcttattccctttctattcgtgattgggatgagataaggggtgacaatgtgaaacactacaaaattaggaaacttgacaatggtggatactatatcacaaccagagcacaatttgatactctgcagaaattggtgaaacactacacagaacatgctgatggtttatgccacaagttgacaactgtgtgtccaactgtgaaacctcagactcaaggtctagcaaaagatgcttgggaaatccctcgagaatctttgcgactagaggttaaactaggacaaggatgtttcggcgaagtgtggatgggaacatggaatggaaccacgaaagtagcaatcaaaacactaaaaccaggtacaatgatgccagaagctttccttcaagaagctcagataatgaaaaaattaagacatgataaacttgttccactatatgctgttgtttctgaagaaccaatttacattgtcactgaatttatgtcaaaaggaagcttattagatttccttaaggaaggagatggaaagtatttgaagcttccacagctggttgatatggctgctcagattgctgatggtatggcatatattgaaagaatgaactatattcaccgagatcttcgggctgctaatattcttgtaggagaaaatcttgtgtgcaaaatagcagactttggtttagcaaggttaattgaagacaatgaatacacagcaagacaaggtgcaaaatttccaatcaaatggacagctcctgaagctgcactgtatggtcggtttacaataaagtctgatgtctggtcatttggaattctgcaaacagaactagtaacaaagggccgagtgccatatccaggtatggtgaaccgtgaagtactagaacaagtggagcgaggatacaggatgccgtgccctcagggctgtccagaatccctccatgaattgatgaatctgtgttggaagaaggaccctgatgaaagaccaacatttgaatatattcagtccttcttggaagactacttcactgctacagagccacagtaccagccaggagaaaatttataattcaagtagcctattttatatgcacaaatctgccaaaatataaagaacttgtgtagattttctacaggaatcaaaagaagaaaatcttctttactctgcatgtttttaatggtaaactggaatcccagatatggttgcacaaaaccacttttttttccccaagtattaaactctaatgtaccaatgatgaatttatcagcgtatttcagggtccaaacaaaatagagctaagatactgatgacagtgtgggtgacagcatggtaatgaaggacagtgaggctcctgcttatttataaatcatttcctttctttttttccccaaagtcagaattgctcaaagaaaattatttattgttacagataaaacttgagagataaaaagctataccataataaaatctaaaattaaggaatatcatgggaccaaataattccattccagttttttaaagtttcttgcatttattattctcaaaagttttttctaagttaaacagtcagtatgcaatcttaatatatgctttcttttgcatggacatgggccaggtttttcaaaaggaatataaacaggatctcaaacttgattaaatgttagaccacagaagtggaatttgaaagtataatgcagtacattaatattcatgttcatggaactgaaagaataagaactttttcacttcagtccttttctgaagagtttgacttagaataatgaaggtaactagaaagtgagttaatcttgtatgaggttgcattgattttttaaggcaatatataattgaaactactgtccaatcaaaggggaaatgttttgatctttagatagcatgcaaagtaagacccagcattttaaaagccctttttaaaaactagacttcgtactgtgagtattgcttatatgtccttatggggatgggtgccacaaatagaaaatatgaccagatcagggacttgaatgcacttttgctcatggtgaatatagatgaacagagaggaaaatgtatttaaaagaaatacgagaaaagaaaatgtgaaagttttacaagttagagggatggaaggtaatgtttaatgttgatgtcatggagtgacagaatggctttgctggcactcagagctcctcacttagctatattctgagactttgaagagttataaagtataactataaaactaatttttcttacacactaaatgggtatttgttcaaaataatgaagttatggcttcacattcattgcagtgggatatggtttttatgtaaaacatttttagaactccagttttcaaatcatgtttgaatctacattcacttttttttgttttcttttttgagacggagtctcgctctgccgcccaggctggagtgcagtggcgcgatctcggctcactgcaagctctgcctcccaggttcacaccattctcctgcctcagcctcccgagtagctgggactacaggtgcccaccaccacgcctggctagttttttgtatttttagtagagacgcagtttcaccgtgttagccaggatggtctcgatctcctgaccttgtgatctgcccgcctcggcctcccaaagtgctgggattacaggcgtgagccaccgcgcccagcctacattcacttctaaagtctatgtaatggtggtcattttttcccttttagaatacattaaatggttgatttggggaggaaaacttattctgaatattaacggtggtgaaaaggggacagtttttaccctaaagtgcaaaagtgaaacatacaaaataagactaatttttaagagtaactcagtaatttcaaaatacagatttgaatagcagcattagtggtttgagtgtctagcaaaggaaaaattgatgaataaaatgaaggtctggtgtatatgttttaaaatactctcatatagtcacactttaaattaagccttatattaggcccctctattttcaggatataattcttaactatcattatttacctgattttaatcatcagattcgaaattctgtgccatggcgtatatgttcaaattcaaaccatttttaaaatgtgaagatggacttcatgcaagttggcagtggttctggtactaaaaattgtggttgttttttctgtttacgtaacctgcttagtattgacactctctaccaagagggtcttcctaagaagagtgctgtcattatttcctcttatcaacaacttgtgacatgagattttttaagggctttatgtgaactatgatattgtaatttttctaagcatattcaaaagggtgacaaaattacgtttatgtactaaatctaatcaggaaagtaaggcaggaaaagttgatggtattcattaggttttaactgaatggagcagttccttatataataacaattgtatagtagggataaaacactaacttaatgtgtattcattttaaattgttctgtatttttaaattgccaagaaaaacaactttgtaaatttggagatattttccaacagcttttcgtcttcagtgtcttaatgtggaagttaacccttaccaaaaaaggaagttggcaaaaacagccttctagcacacttttttaaatgaataatggtagcctaaacttaatatttttataaagtattgtaatattgttttgtggataattgaaataaaaagttctcattgaatgcacctattaaaaaaaaaaaaaaaaaa

SEQ ID NO: 9 - protein, 543 aa, Homo sapiens
MGCIKSKENKSPAIKYRPENTPEPVSTSVSHYGAEPTTVSPCPSSSAKGTAVNFSSLSMTPFGGSSGVTPFGGASSSFSVVPSSYPAGLTGGVTIFVALYDYEARTTEDLSFKKGERFQIINNTEGDWWEARSIATGKNGYIPSNYVAPADSIQAEEWYFGKMGRKDAERLLLNPGNQRGIFLVRESETTKGAYSLSIRDWDEIRGDNVKHYKIRKLDNGGYYITTRAQFDTLQKLVKHYTEHADGLCHKLTTVCPTVKPQTQGLAKDAWEIPRESLRLEVKLGQGCFGEVWMGTWNGTTKVAIKTLKPGTMMPEAFLQEAQIMKKLRHDKLVPLYAVVSEEPIYIVTEFMSKGSLLDFLKEGDGKYLKLPQLVDMAAQIADGMAYIERMNYIHRDLRAANILVGENLVCKIADFGLARLIEDNEYTARQGAKFPIKWTAPEAALYGRFTIKSDVWSFGILQTELVTKGRVPYPGMVNREVLEQVERGYRMPCPQGCPESLHELMNLCWKKDPDERPTFEYIQSFLEDYFTATEPQYQPGENL

SEQ ID NO: 10 - DNA, 25 bp, artificial sequence
  HMG20B PMO antisense oligomer
cgcccagcatcttggtgatctcggg

SEQ ID NO: 11 - DNA, 25 bp, artificial sequence
  HRH1-specific PMO antisense oligo
gcgaaagagcagccgccagttatgg

SEQ ID NO: 12 - DNA, 25 bp, artificial sequence
  NP-specific PMO antisense oligo
cttcataggtgtatccgttctccat

SEQ ID NO: 13 - DNA, 25 bp, artificial sequence
  YES-specific PMO antisense oligo
tttctttacttttaatgcagcccat

SEQ ID NO: 14 - DNA, 25 bp, artificial sequence
  ARF1-specific PMO antisense oligo
atgcttgtggacaggtggaaggaca

SEQ ID NO: 15 - RNA, 21 nt, Homo sapiens
aacgccuuuuggaggugcauc

SEQ ID NO: 16 - RNA, 21 nt, Homo sapiens
aacgccuuuuggaggugcauc

SEQ ID NO: 17 - RNA, 4343 nt, Homo sapiens
  misc_feature (214)..(234): region corresponding to siRNA-214
  misc_feature (318)..(338): region corresponding to siRNA-318
agauuugauaaugggcugcauuaaaaguaaagaaaacaaaaguccagccauuaaauacagaccugaaaauacuccagagccugucaguacaagugugagccauuauggagcagaacccacuacagugucaccauguccgucaucuucagcaaagggaacagcaguuaauuucagcagucuuuccaugacaccauuuggaggauccucagggguaacgccuuuuggaggugcaucuuccucauuuucaguggugccaaguucauauccugcugguuuaacaggugguguuacuauauuuguggccuuauaugauuaugaagcuagaacuacagaagaccuuucauuuaagaagggugaaagauuucaaauaauuaacaauacggaaggagauuggugggaagcaagaucaaucgcuacaggaaagaaugguuauaucccgagcaauuauguagcgccugcagauuccauucaggcagaagaaugguauuuuggcaaaauggggagaaaagaugcugaaagauuacuuuugaauccuggaaaucaacgagguauuuucuuaguaagagagagugaaacaacuaaaggugcuuauucccuuucuauucgugauugggaugagauaaggggugacaaugugaaacacuacaaaauuaggaaacuugacaaugguggauacuauaucacaaccagagcacaauuugauacucugcagaaauuggugaaacacuacacagaacaugcugaugguuuaugccacaaguugacaacuguguguccaacugugaaaccucagacucaaggucuagcaaaagaugcuugggaaaucccucgagaaucuuugcgacuagagguuaaacuaggacaaggauguuucggcgaaguguggaugggaacauggaauggaaccacgaaaguagcaaucaaaacacuaaaaccagguacaaugaugccagaagcuuuccuucaagaagcucagauaaugaaaaaauuaagacaugauaaacuuguuccacuauaugcuguuguuucugaagaaccaauuuacauugucacugaauuuaugucaaaaggaagcuuauuagauuuccuuaaggaaggagauggaaaguauuugaagcuuccacagcugguugauauggcugcucagauugcugaugguauggcauauauugaaagaaugaacuauauucaccgagaucuucgggcugcuaauauucuuguaggagaaaaucuugugugcaaaauagcagacuuugguuuagcaagguuaauugaagacaaugaauacacagcaagacaaggugcaaaauuuccaaucaaauggacagcuccugaagcugcacuguauggucgguuuacaauaaagucugaugucuggucauuuggaauucugcaaacagaacuaguaacaaagggccgagugccauauccagguauggugaaccgugaaguacuagaacaaguggagcgaggauacaggaugccgugcccucagggcuguccagaaucccuccaugaauugaugaaucuguguuggaagaaggacccugaugaaagaccaacauuugaauauauucaguccuucuuggaagacuacuucacugcuacagagccacaguaccagccaggagaaaauuuauaauucaaguagccuauuuuauaugcacaaaucugccaaaauauaaagaacuuguguagauuuucuacaggaaucaaaagaagaaaaucuucuuuacucugcauguuuuuaaugguaaacuggaaucccagauaugguugcacaaaaccacuuuuuuuuccccaaguauuaaacucuaauguaccaaugaugaauuuaucagcguauuucaggguccaaacaaaauagagcuaagauacugaugacagugugggugacagcaugguaaugaaggacagugaggcuccugcuuauuuauaaaucauuuccuuucuuuuuuuccccaaagucagaauugcucaaagaaaauuauuuauuguuacagauaaaacuugagagauaaaaagcuauaccauaauaaaaucuaaaauuaaggaauaucaugggaccaaauaauuccauuccaguuuuuuaaaguuucuugcauuuauuauucucaaaaguuuuuucuaaguuaaacagucaguaugcaaucuuaauauaugcuuucuuuugcauggacaugggccagguuuuucaaaaggaauauaaacaggaucucaaacuugauuaaauguuagaccacagaaguggaauuugaaaguauaaugcaguacauuaauauucauguucauggaacugaaagaauaagaacuuuuucacuucaguccuuuucugaagaguuugacuuagaauaaugaagguaacuagaaagugaguuaaucuuguaugagguugcauugauuuuuuaaggcaauauauaauugaaacuacuguccaaucaaaggggaaauguuuugaucuuuagauagcaugcaaaguaagacccagcauuuuaaaagcccuuuuuaaaaacuagacuucguacugugaguauugcuuauauguccuuauggggaugggugccacaaauagaaaauaugaccagaucagggacuugaaugcacuuuugcucauggugaauauagaugaacagagaggaaaauguauuuaaaagaaauacgagaaaagaaaaugugaaaguuuuacaaguuagagggauggaagguaauguuuaauguugaugucauggagugacagaauggcuuugcuggcacucagagcuccucacuuagcuauauucugagacuuugaagaguuauaaaguauaacuauaaaacuaauuuuucuuacacacuaaauggguauuuguucaaaauaaugaaguuauggcuucacauucauugcagugggauaugguuuuuauguaaaacauuuuuagaacuccaguuuucaaaucauguuugaaucuacauucacuuuuuuuuguuuucuuuuuugagacggagucucgcucugccgcccaggcuggagugcaguggcgcgaucucggcucacugcaagcucugccucccagguucacaccauucuccugccucagccucccgaguagcugggacuacaggugcccaccaccacgccuggcuaguuuuuuguauuuuuaguagagacgcaguuucaccguguuagccaggauggucucgaucuccugaccuugugaucugcccgccucggccucccaaagugcugggauuacaggcgugagccaccgcgcccagccuacauucacuucuaaagucuauguaaugguggucauuuuuucccuuuuagaauacauuaaaugguugauuuggggaggaaaacuuauucugaauauuaacgguggugaaaaggggacaguuuuuacccuaaagugcaaaagugaaacauacaaaauaagacuaauuuuuaagaguaacucaguaauuucaaaauacagauuugaauagcagcauuagugguuugagugucuagcaaaggaaaaauugaugaauaaaaugaaggucugguguauauguuuuaaaauacucucauauagucacacuuuaaauuaagccuuauauuaggccccucuauuuucaggauauaauucuuaacuaucauuauuuaccugauuuuaaucaucagauucgaaauucugugccauggcguauauguucaaauucaaaccauuuuuaaaaugugaagauggacuucaugcaaguuggcagugguucugguacuaaaaauugugguuguuuuuucuguuuacguaaccugcuuaguauugacacucucuaccaagagggucuuccuaagaagagugcugucauuauuuccucuuaucaacaacuugugacaugagauuuuuuaagggcuuuaugugaacuaugauauuguaauuuuucuaagcauauucaaaagggugacaaaauuacguuuauguacuaaaucuaaucaggaaaguaaggcaggaaaaguugaugguauucauuagguuuuaacugaauggagcaguuccuuauauaauaacaauuguauaguagggauaaaacacuaacuuaauguguauucauuuuaaauuguucuguauuuuuaaauugccaagaaaaacaacuuuguaaauuuggagauauuuuccaacagcuuuucgucuucagugucuuaauguggaaguuaacccuuaccaaaaaaggaaguuggcaaaaacagccuucuagcacacuuuuuuaaaugaauaaugguagccuaaacuuaauauuuuuauaaaguauuguaauauuguuuuguggauaauugaaauaaaaaguucucauugaaugcaccuauuaaaaaaaaaaaaaaaaaa

(19) United States
(12) Patent Application Publication
(10) Pub. No.: US 2007/0087982 A1
(43) Pub. Date: Apr. 19, 2007

(54) Methods of treatment and diagnosis using modulators of virus-induced cellular gene sequences

(76) Inventors: Jay A. Nelson, Tualatin, OR (US); Alec Hirsch, Portland, OR (US); Klaus Fruh, Portland, OR (US); Victor DeFilippis, Hillsboro, OR (US); Jeffrey S. King, Portland, OR (US); Ashlee V. Moses, Portland, OR (US); Laura Jelinek, Tigard, OR (US)

Correspondence Address: KLARQUIST SPARKMAN, LLP, 121 SW SALMON STREET, SUITE 1600, PORTLAND, OR, 97204, US

(21) Appl. No.: 10/564,369

(22) PCT Filed: Apr. 15, 2004
(86) PCT No.: PCT/US04/11988

§ 371 Date: Aug. 23, 2006

Related U.S. Application Data
(60) Provisional application No. 60/486,694, filed on Jul. 11, 2003.
(60) Provisional application No. 60/533,103, filed on Dec. 29, 2003.

(51) Int. Cl. A61K 48/00; A61K 31/675; A61K 31/519; A61K 31/405

(52) U.S. Cl. 514/44; 514/81; 514/262.1; 514/265.1; 514/414; 514/264.11

(57) ABSTRACT

Applicants have used microarrays, gene expression profiling, and gene silencing methods to identify and provide a plurality of ‘validated’ virus-induced cellular gene sequences (e.g., HMG20B, HRH1, NP and c-YES (src family kinases)) and pathways useful as therapeutic targets for modulation of viral-mediated cellular effects. Particular embodiments provide therapeutic compositions, and methods for modulation of viral infection, replication, maturation, progression, or other virally-related conditions or diseases, comprising inhibition of virally-induced gene sequences and gene products. Additional embodiments provide screening assays for compounds useful to modulate viral infection, replication, maturation or progression, or viral-related conditions or diseases. Further embodiments provide diagnostic and/or prognostic assays for viral infection, replication, maturation or progression. Preferably, the viruses all selected from the group consisting of retroviruses (e.g., human immunodeficiency virus (HIV), and viruses of the family Flaviviridae that includes the flaviviruses (e.g., West Nile virus (WNV), Japanese encephalitis virus (JEV), yellow fever virus (YFV) and Dengue fever virus (DEN)), and hepatitis C virus (HCV).

### CROSS REFERENCE TO RELATED APPLICATIONS

This application claims benefit of priority to U.S. Provisional Patent Application Ser. Nos. 60/486,694, filed 11 Jul. 2003, and 60/533,103, filed 29 Dec. 2003, both incorporated by reference herein in their entirety.

### STATEMENT REGARDING FEDERALLY SPONSORED RESEARCH

This work was partially funded by NIH/NIAID grant number 1R41 AIo55218-01, and the United States government has, therefore, certain rights to the present invention.

### FIELD OF THE INVENTION

The present invention relates to the identification and use, including therapeutic use, of modulators of virus-induced gene expression, including, but not limited to, modulators of cellular genes or gene products induced by human immunodeficiency virus (HIV), and viruses of the family Flaviviridae, which includes the flaviviruses (e.g., West Nile virus (WNV), Japanese encephalitis virus (JEV), yellow fever virus (YFV) and Dengue fever virus (DEN)), as well as hepatitis C virus (HCV). In particular embodiments, the invention relates to the novel use of inhibitors of src-family kinases for inhibition of viral progression, maturation or infection.

### BACKGROUND

Human immunodeficiency virus (HIV). The AIDS epidemic continues to spread, and urgent efforts are required to stem this global health crisis. While modern antiretroviral drugs have enabled many HIV-positive individuals to live longer and delay progression to AIDS, these drugs do not ultimately cure infection, and long term use is associated with toxicity and the emergence of drug resistant strains. Cost and delivery issues also make such therapies prohibitive in much of the developing world, and the development of an effective vaccine has, at least to date, proved elusive.

Significantly, the high mutation rate of HIV enables immune escape, as well establishment of eradication-resistant latent reservoirs, favoring persistence of the virus despite immunization or antiretroviral therapy. Another reason for the success of HIV as an intracellular pathogen is its remarkable ability to exploit the molecular machinery of the host cell to facilitate persistence, replication and spread of the virus. The virus has likely evolved mechanisms to modulate expression levels of these cellular co-factors to promote its life cycle.

Therefore, there is a strong need in the art to disrupt key interactions between HIV and the host cell facilitators of infection and viral replication by counteracting the viral modulation of those genes. Central to the success of this approach however is the initial identification of these cellular co-factors and clarification, and further, validation, of their role in the virus life cycle.

Limited prior art identification. Sheehy et al. (Nature 418:646-650, 2002) describe the identification of an anti-HIV cellular factor that is thought to be the human cell target of the HIV-vif protein. This protein, named CEM15, possesses significant amino acid similarity to the mRNA-editing cytidine deaminase apobec-1. Although wild type levels of vif are sufficient to overcome the action of CEM15, this factor renders vif-defective virions non-infectious, and thus expression of a vif-resistant form of this protein may be of therapeutic interest. Significantly, however, the studies of Sheehy et al were limited by their utilization of a PCR-based cDNA subtraction strategy to identify CEM15. Additionally, there is a need for further validation of such potential therapeutic targets.

There is, therefore, a need in the art for additional methods and studies to distinguish, from among those HIV-regulated cellular gene sequences, those actually required for HIV-induced proliferative and phenotypic/developmental changes and which could therefore provide validated intervention targets for the inhibition of HIV-induced cellular phenomena and the treatment of HIV-related diseases and hyperproliferative disorders such as cancer. There is a need in the art for compositions and methods to affect such validated targets, and for screening a diagnostic assays premised on such validated HIV-relevant targets.

Flaviviruses. The family Flaviviridae includes the flaviviruses, hepatitis C virus (HCV), the animal pathogenic pestiviruses, and likely the GB virus A (GBV-A), GBV-B and GBV-C/hepatitis G viruses (Murphy et al., Virus Taxonomy. Sixth Report of the International Committee on Taxonomy of Viruses, pp. 424-426, 1995; Vienna & New York: Springer-Verlag.

The prototypical flavivirus, yellow fever virus (YFV), was first isolated in 1927. Since that time, the membership of the genus Flavivirus has grown to over 70 known viruses, of which more than half are associated with human disease. These include West Nile virus (WNV), Japanese encephalitis virus (JEV), St Louis encephalitis (SLE), and Dengue (DEN). The majority of the flaviviruses are vector borne, with approximately 50% transmitted by mosquitoes, and 30% carried by ticks. The remaining 20% are classified as “non-vector,” which are transmitted by an as yet unidentified vector or zoonotically from rodents or bats (e.g., see 2001 review by Burke & Monath; Flaviviruses, p. 1043-1125; in D. M. Knipe, and P. M. Howley (eds), Knipe, D. M. Howley, P. M., Fourth ed. Lippincott Williams & Wilkins, Philadelphia). The general transmission cycle of the vector borne viruses involves the acquisition of the virus by the arthropod through feeding on an infected host; typically birds, small mammals, or primates. The virus replicates in the insect host, which in turn can infect an immunologically naïve bird (or small mammal or primate, depending on the virus).

In the case of WNV, human infection through the bite of an infected mosquito results in fever in 20 percent of cases, and 1 in 150 infections result in neurological disease. The greatest risk factor for neurological disease following infection appears to be advanced age. Treatment for WNV is supportive, there being neither a widely approved therapy available, nor a vaccine approved for use in humans, although a vaccine consisting of formalin-inactivated WNV is approved for veterinary use in horses.

The flaviviruses have been sub-classified on the basis of antigenic relatedness, or more recently, on sequence similarity. Sequence information has been used to classify the viruses into 14 clades, which correlate closely with the previous antigenic classifications (Kuno, et al., J Virol. 72:73-83, 1998).

Epidemiology and pathology of flaviviruses. A large number of flaviviruses are associated with human disease, and the epidemiology and pathology of three of these, West Nile virus (WNV), Dengue virus (DEN), and Japanese Encephalitis virus (JEV), are briefly summarized here.

West Nile Virus (WNV) is a mosquito borne pathogen associated with fever and encephalitis. WNV was first identified in Uganda in 1937 (Smithburn, et al., 1940. American Journal of Tropical Medicine, 20:471-92, 1940). Although outbreaks of WNV have been sporadic and associated with mild illness since its discovery, the frequency and severity of WNV disease, in horses as well as in humans, has increased since the mid 1990s (Petersen & Roehrig, Emerg Infect Dis, 7:611-4, 2001). Outbreaks have occurred in Romania (1996), Morocco (1996), Tunisia (1997), Italy (1998), Russia (1999), Israel (1999 and 2000) and the U.S. (1999, and each summer since). The outbreak in New York in 1999 appears to mark the beginning of the spread of WNV throughout the U.S. In 1999, there were a total of 62 reported human cases isolated to the state of New York, 59 of which required hospitalization. In 2000, there were 21 cases in three states, increasing to 66 cases in ten states in 2001. The CDC, in 2002, reported 3,580 laboratory-positive human cases over 38 states (anonymous 2002 on-line posting date, West Nile Virus, Centers for Disease Control). If the spread of the virus is measured by the presence of infected birds or mosquitoes, the geographic extent is even greater, likely encompassing 43 states. It is expected that the virus will spread further. Transmission involves cyclic transfer from mosquitoes of the genus Culex to birds and back. Humans and horses are dead-end hosts (Campbell, et al., Lancet Infect Dis. 2:519-29, 2002).

As stated above, approximately 20% of individuals infected with WNV develop fever, as estimated by a serological survey conducted subsequent to the 1999 New York outbreak (Mostashari, et al., Lancet, 358:261-4, 2001). This study estimates that the total number of infections during this period was 8,200 of which 62 were reported. The fever is sometimes accompanied by weakness, nausea, headache, myalgia, arthralgia, and rash. About 1 in 150 infections results in neurological disease such as encephalitis or meningitis (Mostashari, et al., Lancet, 358:261-4, 2001; Petersen & Marfin, Ann Intern Med. 137:173-9, 200). Of the 59 WNV patients hospitalized in New York in 1999, 54 were diagnosed with encephalitis or meningitis; 12% of these hospitalized patients later died. In 2002, 211 of the reported cases resulted in death (approximately a 6% fatality rate). The greatest risk factor for death is advanced age (Nash, et al., N Engl J. Med. 344:1807-14, 2001). Significantly, there are currently no approved antiviral therapies for WNV; treatment is supportive.

Dengue Virus (DEN). Dengue virus infects approximately 100 million people a year. It is endemic in virtually all the tropic areas of the world. There are four serotypes of DEN (Dengue type 1-4). All are spread primarily by the mosquito Aedes Aegypti, which lives in close proximity to humans (i.e. a “domestic” mosquito). Unlike the case for most flaviviruses, humans are a natural host for dengue, and can produce high enough titers in the blood to continue the transmission cycle (Burke & Monath, 2001, supra; Gibbons & Vaughn, Bmj. 324:1563-6, 2002; and Solomon & Mallewa 2001. J Infect. 42:104-15, 2001).

DEN infection may result in one of several syndromes (McBride & Bielefeldt-Ohmann, Microbes Infect. 2:1041-50, 2000). Dengue infection is characterized by fever, headache and rash. A more severe form, Dengue hemorrhagic fever (DHF) may include increased vascular permeability and leakage of plasma from blood vessels into tissue. Mild hemorrhage may also occur. DHF is graded on a scale of I through IV. Grade II includes greater bleeding (gum, nose, GI tract), while grades III and IV feature increased vascular leakage, accompanied by loss of blood pressure and shock. Grades III and IV are also known as Dengue shock syndrome. DHF is more likely to occur when DEN infection is followed by a second infection of a different serotype. This may be due to the presence of circulating antibody that reacts with, but does not neutralize, the second infecting strain. The presence of these antibodies allows antibody-dependent enhancement of infection of macrophages, which take up antibody-bound DEN via their Fc receptors. It is postulated that macrophage infection results in increased T-cell activation and cytokine production, leading to severe immunopathology (Halstead, S. B., Science 239:476-81, 1988). This model does not explain, however, the relative rarity of DHF even in patients experiencing a second DEN infection, or the occasional appearance of DHF during primary DEN infection. Other theories of DHF pathogenesis include the possibility of virulence factors present only in specific DEN strains or “quasispecies,” or the possibility of an autoimmune response elicited by the similarity of DEN antigens to various human clotting factors (Bielefeldt-Ohmann, H., Trends Microbiol. 5:409-13, 1997; Leitmeyer, et al., J. Virol. 73:4738-4, 1999; and Markoff, et al., J Infect Dis. 164:294-30, 1991).

Japanese Encephalitis Virus (JEV). JEV is endemic in much of Southeast Asia, ranging from Japan and Korea at its northern range, to India in the west, and Indochina and Indonesia to the South. Sporadic cases have also been reported as far south as Papua New Guinea and Australia. Annually, there are approximately 35,000 cases and 10,000 deaths, and these figures may underestimate the true toll of the disease due to incomplete surveillance and reporting. JEV is a member of an antigenic complex and clade that also include WNV. It is spread primarily by the mosquito Culex tritaeniorhynchus, cycling through its natural viremic hosts, pigs and birds.

Most JEV infections are sub-clinical, with only 1 in 250 infections resulting in symptoms. The primary clinical manifestation is encephalitis. Symptoms begin with headache, fever, and gastrointestinal problems after a 5-15 day incubation period. These symptoms may be followed by irritability, nausea, and diarrhea with decline to generalized weakness, stupor, or coma. In children, seizures are common, and 5-30% of such cases are fatal.

Significantly, there is no specific treatment for JEV, other than supportive care. However, vaccines do exist for JEV. These include a formalin-inactivated vaccine, as well as a live attenuated strain. The inactivated version has been used widely in Japan and China since the 1960s, and is also licensed for use in the U.S. and Europe for those traveling to areas in which JEV is endemic. The attenuated virus has also seen wide use in China. Both vaccines, when delivered with appropriate booster regimens, have shown efficacies greater than 90% (Burke & Monath, supra; Tsai, et al., 1999, Japanese Encephalitis Vaccines, p. 672-710; in S. A. Plotkin, and W. A. Orenstein (eds), Vaccines. W.B. Saunders Company, Philadelphia).

Flavivirus replication. The flaviviruses are small enveloped viruses that contain a single, positive-sense RNA genome of approximately 11 kilobases (kb). The RNA is capped at its 5′ end, but not 3′ polyadenylated. The RNA encodes a single large open reading frame (ORF) that is processed into 10 subunits that comprise the structural components of the virion and the viral replication complex (Lindenbach & Rice, 2001, Flaviviridae: The Viruses and Their Replication, p. 991-1041; in D. M. Knipe, and P. M. Howley (eds), c, Fourth ed. Lippincott Williams & Wilkins, Philadelphia). The flaviviruses all possess a common organization to the coding sequence of the genome. The structural subunits are located at the 5′ end. These include the core (C), membrane (prM/M), and envelope (E) proteins. These are followed by the non-structural proteins NS1, NS2A, NS2B, NS3, NS4A, NS4B, and NS5. NS2B and NS3 function as the serine protease that is responsible for processing much of the viral polyprotein. NS5, the most highly conserved of the flavivirus proteins, acts as the RNA dependent RNA polymerase necessary for viral replication, and may also function as a methyltransferase that provides the genomic 5′ cap. The other members of the non-structural group are largely hydrophobic and of unknown function (Id).

Flavivirus infection of the host cell begins via attachment of the E-protein to a cellular receptor. Definitive identification of a receptor for any of the flavivirus species is still absent, but glycosaminoglycans appear to be involved in the initial attachment (Chen, et al., Nat. Med. 3:866-71, 1997). Entry of the virus into the host cell probably occurs by receptor mediated endocytosis, followed by low-pH dependent fusion of the virion with the endosome membrane, releasing the nucleocapsid and genomic RNA into the cytoplasm (Lindenbach & Rice, 2001, supra; Kuhn, et al., Cell. 108:717-2, 2002).

Translation of the RNA by the host cell follows, and the polyprotein is cleaved into its constituent subunits by a combination of a host cell ER resident protease and the NS2B/NS3 virally encoded serine protease. Replication of the genomic RNA occurs through a negative sense intermediate, and can be detected as early as three hours after infection in the case of YFV. Flavivirus infection induces a proliferation of ER membranes in the host cell and the formation of “smooth membrane structures,” that are groups of vesicle-like structures in the ER lumen. The smooth membrane structures co-localize with double-stranded RNA (presumably the replicative intermediate), as well as NS1 and NS3, and are believed to be the sites of RNA replication. NS2B and NS3, the constituents of the viral protease, localize to an adjacent region of induced membranes (dubbed “convoluted membranes”), suggesting that polyprotein processing and NA replication are spatially separated within the infected cell (Westaway, et al., J Virol. 71:6650-61, 1997).

Assembly and release of virions largely remains a black box. Cis-acting packaging signals in the RNA have not been identified, although the viral nucleocapsid protein C has been shown to interact with the 5′ and 3′ ends of the genome (Khromykh & Westaway, Arch Virol. 141:685-99, 1996). The envelope is most likely acquired by budding of the nucleocapsid precursor into the ER. At a later point in virus maturation, the prM protein is cleaved into the mature form (M) by the cellular protease furin (Stadler, et al., J Virol. 71:8475-81, 1997). It is currently believed that prM functions to prevent the E protein from undergoing the low pH dependent conformational change while in the cell. In agreement with this hypothesis, prevention of prM cleavage results in the release of virus particles that are less infectious than wild-type (Heinz & Allison, Adv Virus Res. 55:231-69, 2000).

Infection of the host is thought to begin in the Langerhans cells of the skin following the bite of a carrier arthropod. Viral replication continues in the regional tissue and lymph nodes, which results in the dissemination of the virus into the bloodstream. Replication then proceeds at several sites, including connective tissue, smooth muscle, liver and spleen. Neural invasion appears to occur through the olfactory epithelium in experimentally infected rodents. It is unclear if this is the primary route used by virus to gain access to the CNS in infected humans (McMinn et al., Virology. 220:414-23, 1996; Monath, et al., Lab Invest. 48:399-410, 1983).

Treatment and vaccine development. Treatment for most flavivirus infections resulting in disease is supportive (e.g., fluid management, mechanical ventilation, transfusion in case of severe hemorrhage, etc.). Recent reports show efficacy of ribavirin and interferon-α2b in WNV infection, although controlled clinical trials have not been completed (Petersen & Marfin, Ann Intern Med. 137:173-9, 2002). Preventive vaccines exist for YFV and JEV, both based on live, attenuated strains. Similar strategies as well as the construction of chimeric viruses based on the backbones of approved flavivirus vaccines are being used to develop vaccines against WNV, Dengue, and others (Monath, T. P., Ann N Y Acad Sci. 951:1-12, 2001).

There is, therefore, a pronounced need in the art for novel therapeutic methods and compositions having utility for preventing or inhibiting flavivirus infection, and for treatment and/or prevention of conditions related to flavivirus infection.

Hepatitis C Virus (HCV). Hepatitis C Virus (HCV) is closely related to flaviviruses; belonging to same Flaviviridae family.

There is, additionally, a pronounced need in the art for novel therapeutic methods and compositions having utility for preventing or inhibiting HCV infection, and for treatment and/or prevention of conditions related to HCV infection.

### SUMMARY OF THE INVENTION

The present invention relates to the identification and use, including therapeutic use, of modulators of virus-induced gene expression, including, but not limited to, modulators of cellular genes induced by human immunodeficiency virus (HIV), flaviviruses (e.g., West Nile virus (WNV), Japanese encephalitis virus (JEV), St. Louis encephalitis (SLE), yellow fever virus (YFV) and Dengue fever virus (DEN)) and hepatitis C (and G) virus (HCV; closely related to flaviviruses). Preferred modulators are inhibitors capable of reducing the expression of virus (e.g., retroviral, flavivirus or flavivirus-related viruses; e.g., HIV, WNV, JEV, SLE, YFV, DEN and HCV)-induced cellular genes, reducing or preventing the expression of mRNA from such virus-induced genes, or reducing the biological activity of such corresponding virus-induced cellular gene products. The invention provides therapeutic methods, diagnostic methods and compositions useful for the treatment of virus-related disorders and disease. Particular embodiments also provide drug candidate screening assays.

In preferred embodiments, the invention relates to the novel use of inhibitors of src-family kinases for treatment or inhibition of viral (e.g., HIV, WNV, JEV, SLE, YFV, DEN and HCV) progression, maturation, infection, etc. Preferably, the inventive methods and compositions are directed to inhibition of the src family kinase c-yes.

Preferably the retrovirus is HIV. Preferably, the flavivirus is selected from the group consisting of West Nile virus (WNV), Japanese encephalitis virus (JEV), St. Louis encephalitis (SLE), and Dengue (DEN). Preferably, the flavivirus is WNV. Preferably, the flavivirus-related virus is HCV.

Particular embodiments provide modulators of virus (e.g., HIV, WNV, JEV, SLE, YFV, DEN and HCV)-induced cellular gene expression including, but not limited to, antisense molecules, siRNA agents, ribozymes, antibodies or antibody fragments, proteins or polypeptides as well as small molecules. The inventive modulators are useful for reducing the expression of such virus-induced genes, reducing or preventing the expression of mRNA from such virus-induced genes, or reducing the biological activity of such corresponding virus-induced cellular gene products. Preferably, the inventive modulators are directed to one or more validated virus (e.g., HIV, WNV, JEV, SLE, YFV, DEN and HCV)-induced gene targets, the expression of which is required, at least to some extent, for respective virus infection, replication, maturation or progression, and corresponding virus-mediated cellular effects, conditions and diseases.

Particular embodiments of the present invention provide therapeutic methods and compositions for modulation of virus (e.g., retroviral, flavivirus or flavivirus-related viruses; e.g., HIV, WNV, JEV, SLE, YFV, DEN and HCV) infection, replication, maturation or progression comprising use of inventive modulators for inhibition of the expression of such virus-induced cellular genes, reducing or preventing the expression of mRNA from such virus-induced genes, or reducing the biological activity of such corresponding virus-induced cellular gene products.

Preferred inventive modulators are small molecule modulators, and induced cellular gene-specific oligonucleotides (such as antisense molecules; e.g., phosphorodiamidate morpholino oligomers; PMOs), ribozymes, RNA interference (siRNA) methods and agents, for targeting and/or modulating the expression of polynucleotides (e.g., mRNA) comprising HIV-induced cellular gene sequences.

Preferred antisense molecules or the complements thereof comprise at least 10, 15, 17, 20, 22 or 25 consecutive complementary nucleotides of, or hybridize under stringent or highly stringent conditions to at least one of the nucleic acid sequences from the group consisting of SEQ ID NOS:1, 3, 5, 6, and 8, and complements thereof. Preferably, such antisense molecules are PMO antisense molecules.

Preferred compositions comprise one or more of such modulators or preferred modulators, along with a pharmaceutically acceptable carrier, excipient or diluent.

Additional embodiments provide screening assays for compounds useful to modulate viral (e.g., HIV, WNV, JEV, SLE, YFV, DEN and HCV) infection. Preferably, such screening assays are based on the use of respective validated viral-induced cellular gene targets. Preferred embodiments provide a method for identification of agents having potential therapeutic utility for the treatment of virus infection and virus-related conditions, comprising: obtaining cells suitable to support a viral (e.g., HIV, WNV, JEV, SLE, YFV, DEN and HCV) infection; infecting the cells with a virus; contacting the infected cells with an agent that inhibits particular viral-induced cellular gene expression and/or activity (e.g., a src family kinase inhibitor); and determining whether the respective viral infection is diminished in some respect relative to control infected cells that are not contacted by the agent, whereby potential therapeutic agents are, at least in part, identified.

Preferably, the cells suitable to support flavivirus or hepatitis C virus (HCV) infection are selected from the group consisting of primary human hepatocellular carcinoma derived cells or cell-lines derived therefrom, Huh 7 cells, neuroblastoma cells or cell-lines derived therefrom, SKN-MC cells, peripheral blood mononuclear cells (PBMC), macrophages, endothelial cells, and combinations thereof. Preferably, the cells suitable to support flavivirus or HCV infection are selected from the group consisting of primary human hepatocellular carcinoma derived cells or cell-lines derived therefrom, Huh 7 cells, neuroblastoma cells or cell-lines derived therefrom, SKN-MC cells, and combinations thereof.

Preferably, the cells suitable to support HIV infection are selected from the group consisting of MT-2 cells (human T cell leukemia cell line MT-2), THP-1 cells (human myeloid (monocyte/macrophage) cell line derived from an acute monocytic leukemia), T-cells, endothelial cells, and combinations thereof. Preferably, the cells suitable to support HIV infection are selected from the group consisting of MT-2 cells, THP-1 cells, and combinations thereof.

In particular embodiments, viral (e.g., HIV, WNV, JEV, SLE, YFV, DEN and HCV) infection precedes contacting of the cells with the agent. Alternatively, in other embodiments, such viral infection is subsequent to contacting of the cells with the agent.

Further embodiments provide a method for inhibiting viral (e.g., HIV, WNV, JEV, SLE, YFV, DEN and HCV) infection, replication, maturation or progression, comprising administration of a src family kinase inhibitor selected from the group consisting of src family kinase-specific antisense RNA, src family kinase-specific siRNA, and small molecule inhibitors of a src family kinase. Preferably, the src family kinase is c-yes kinase.

Additional embodiments provide a method for the treatment of flavivirus infection and related conditions, comprising administration, to a subject in need thereof, of a therapeutically effective amount of an inhibitor of viral (e.g., HIV, WNV, JEV, SLE, YFV, DEN and HCV)-induced cellular gene expression (e.g., an inhibitor of src family kinase expression or activity), whereby virus infection, replication, maturation or progression, and/or related conditions are diminished relative to non-treated subjects. Preferably, the inhibitor is selected from the group consisting of src family kinase-specific antisense RNA, src family kinase-specific siRNA, and a small molecule inhibitor of a src family kinase. Preferably, the src family kinase is c-yes kinase.

Preferably, the small molecule src family kinase inhibitor is a compound having the structure of Formula I, Formula I(b), or Formula II, or salts thereof: 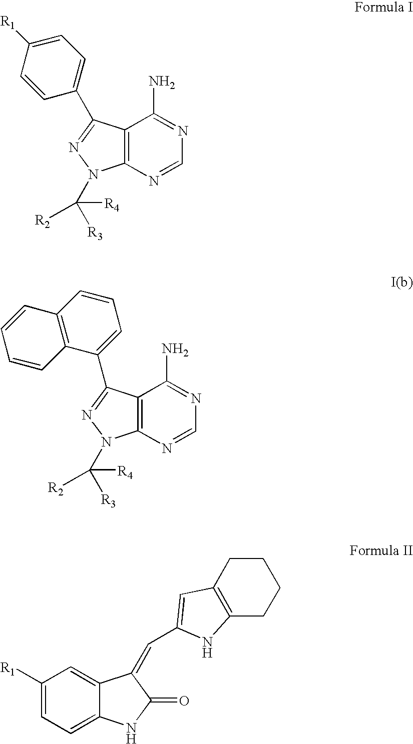
Preferably, for Formula I or I(b), R₁ is halogen or methyl, and R₂, R₃ and R₄ are independently a C1-C3 straight or branched alkyl. Preferably, for Formula I, R₁ is —SO₂N(CH₃)₂, or —SO₂NH₂. Preferably, the inhibitor is 4-Amino-5-(4-chlorophenyl)-7-(t-butyl)pyrazolo[3,4-d]pyrimidine (“PP2”). Preferably, the inhibitor of Formula I(b) is 4-Amino-1-tert-butyl-3-(1′-naphthyl)pyrazolo[3,4-d]pyrimidine (Calbiochem/Merck Biosciences; 529579 PP1 analog) (Bishop, A. C., et al., Nature 407:395, 2000; and Bishop, A. C., et al., J. Am. Chem. Soc. 121:627, 1999). Preferably, for Formula II, the inhibitor is 2-oxo-3-(4,5,6,7-tetrahydro-1H-indol-2-ylmethylene)-2,3-dihydro-1H-indole-5-sulfonic acid dimethylamide (“SU6656”).

In additional preferred embodiments, the SFK inhibitor is a tetrahydroindole-based indolinone, or a pharmaceutically acceptable salt thereof (SUGEN, San Francisco, Calif.) (see Tables 1 and 2 of Guan et al., Bioorg. Med. Chem. Lett. 14:187-190, 2004; incorporated by reference herein in its entirety).

Preferably, the tetrahydroindole-based indolinone is a substituted 3-[3-(3-dimethylamino-propyl)-4,5,6,7-tetrahydro-1H-indol-2-ylmethylene]-1,3-dihydro-indole-2-one. Preferably, the tetrahydroindole-based indolinone is 3-[3-(3-dimethylamino-propyl)-4,5,6,7-tetrahydro-1H-indol-2-ylmethylene]-2-oxo-2,3-dyhydro-1H-indole-5-sulphonic acid methylamide (compound 1a of Guan et al., 2004, supra).

Preferably, in these compounds, there is a sulfone group directly attached to the C-5 position of the oxindole ring (i.e., to the oxindole core). Preferably, the C-3′ position of the tetrahydroindole core is substituted (Formula III): 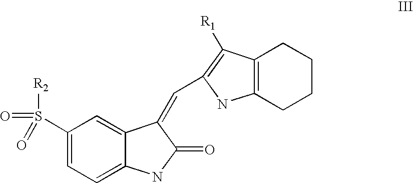
wherein R₂ is C₂H₅ or NHR₃, wherein R₃ is a C1 to C3 linear or branched alkyl moiety, and wherein R₁ is independently —(CH₂)₃N(CH₃)₂, —CH₂N(CH₂CH₂)₂O, —(CH₂)₂N(CH₂CH₂)₂O, —(CH₂)₃N(CH₂CH₂)₂O, or —(CH₂)₃N(CH₂CH₂)₂N CH₃.

Preferably, the substituents at the C-5 position of the oxindole ring are 5-ethylsulfone, or 5-methylsulfonaamide.

Preferably, the C-3′ position substituent is a basic (3-amino)propyl substituent. Preferably, the C-3′ position basic (3-amino)propyl substituent is: —(CH₂)₃N(CH₃)₂; —(CH₂)₃N(CH₂CH₂)₂O; or —(CH₂)₃N(CH₂CH₂)₂NCH₃.

Preferably the tetrahydroindole-based indolinone compound is compound: 1a [R₂ is NH CH₃ and R₁ is —(CH₂)₃N(CH₃)₂]; 1i [R₂ is C₂H₅, and R₁ is —(CH₂)₃N(CH₃)₂]; 3f [R₂ is NH CH₃ and R₁ is —(CH₂)₃N(CH₂CH₂)₂O]; 3g [R₂ is NH CH₃ and R₁ is —(CH₂)₃N(CH₂CH₂)₂N CH₃]; or 4g [R₂ is C₂H₅, and R₁ is —(CH₂)₃N(CH₂CH₂)₂N CH₃] of Guan et al., 2004, supra.

In alternate preferred embodiments, the SFK inhibitor is compound with a pyrido-[2,3-d]pyrimidine core structure, or a pharmaceutically acceptable salt thereof. Preferably, the pyrido-[2,3-d]pyrimidine compound is PD173955 (Formula IV) (Parke-Davis/Warner-Lambert, Ann Arbor, Mich.) (see FIG. 1 of Moasser et al., Cancer Research 59:6145-6152, 1999; incorporated by reference herein in its entirety):

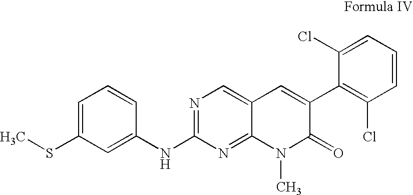

In yet additional preferred embodiments, the SFK inhibitor is a substituted 5,7-diphenyl-pyrolo[2,3-d]pyrimidine, or a pharmaceutically acceptable salt thereof (Novartis, Basel, Switzerland) (see FIG. 1 of Missbach et al., Bone 24:437-449, 1999; incorporated by reference herein in its entirety).

Preferably, the substituted 5,7-diphenyl-pyrolo[2,3-d]pyrimidine is one of the compounds 1-8 of Missbach et al (Id) according to Formula V:

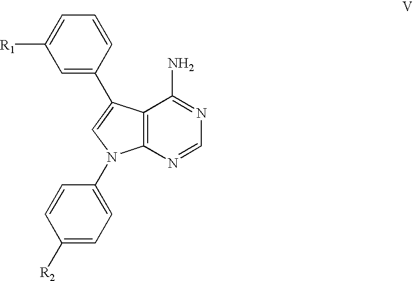

Wherein R₁ is either H or —OCH₃, and wherein R₂ is independently —(CH₂)₂OH, —CH₂COOH, —(CH₂)₂N(CH₃)₂, —(CH₂)₂NH(CH₂)₂OH, —(CH₂)₂NCH₃(CH₂)₂OCH₃, —CH₂)₂N(CH₂CH₂)₂NCH₃, or —(CH₂)₂N(CH₂CH₂)₂CHOH.

Preferably the substituted 5,7-diphenyl-pyrolo[2,3-d]pyrimidine is CGP77675 (i.e., where R₁ is —OCH₃R₂, and R₂ is —(CH₂)₂N(CH₂CH₂)₂CHOH; compound 8 of Missbach et al) (Id).

Further embodiments provide diagnostic or prognostic assays for viral (e.g., HIV, WNV, JEV, SLE, YFV, DEN and HCV) infection, replication, maturation or progression, and related conditions and disorders.

### BRIEF DESCRIPTION OF THE DRAWINGS

FIG. 1A shows the potency of concentrated HIV virus (89.6) stocks using MAGI cells infected with 1 μl, or 0.1 μl of concentrated virus (leftmost panel, and center panel, respectively), as compared to 1 μl of unconcentrated virus (rightmost panel). The left (0.1 μl) and center (1 μl) panels show that the number of blue-staining cells increases in a dose-responsive manner with the use of concentrated HIV-1 89.6 stocks.

FIG. 2A shows, by fluorescence microscopy (left panel), MT-2 cells (human T cell leukemia cell line) efficiently loaded with a FITC-tagged phosphorodiamidate morpholino oligomer (PMO). The right panel shows control cells (treated EPEI without the PMO).

FIG. 2B shows typical, extensive HIV-induced syncytia in control (no PMO anti-sense oligomer) MT-2 cells at 48 hpi hours post-infection).

FIG. 3A illustrates curves showing inhibition of HIV replication in HIV-infected THP-1 cells by PMOs specific for particular HIV-induced cellular genes. The HIV-1 P24 ELISA assay monitors HIV p24 gag production (in pg/ml along the Y-axis) by PMO-treated HIV-infected THP-1 cells. The X-axis shows time. PMOs corresponding to HMG20, HRH-1, NP and c-YES were particularly effective at reproducibly inhibiting HIV replication.

FIG. 3B illustrates curves showing inhibition of HIV replication in HIV-infected MT-2 cells by PMOs specific for particular HIV-induced cellular genes. The HIV-1 P24 ELISA assay monitors HIV p24 gag production (in pg/ml along the Y-axis) by PMO-treated HIV-infected MT-2 cells. The X-axis shows time. As in the case of HIV-infected THP-1 cells (FIG. 3A), PMOs corresponding to HMG20, HRH-1, NP and c-YES were particularly effective at reproducibly inhibiting HIV replication.

FIG. 4 shows inhibition of HIV p24 production in MT-2 cells infected with HIV 89.6 in the continued presence (10 μM) of the src family kinase inhibitor PP2 (4-Amino-5-(4-chlorophenyl)-7-(t-butyl)pyrazolo[3,4-d]pyrimidine). PP3, and DMSO correspond to inactive analog, and carrier controls, respectively. The X-axis shows time. The HIV-1 P24 ELISA assay monitors HIV p24 gag production (in pg/ml along the Y-axis) by the various treated HIV-infected MT-2 cells.

FIG. 5 shows that c-Yes-siRNA molecules inhibited c-YES as well as HIV replication. The left panel shows p24 ELISA assay results of HIV infected THP-1 cells treated with siRNA (316 c-Yes siRNA, from Dharmacon, Lafayette, Colo.). Time post-infection is plotted on the x-axis, and optical density at 450 nm (a measure of p24 production as described herein) is plotted on the y-axis. The right panel shows a Western blot analysis of a SDS-PAGE gel, using antibodies to c-Yes and paxillin (as a cellular control). For this analysis, Huh7 cells were transfected with: c-Yes-specific siRNA (rightmost lane); m33 (negative control; second lane from right), transfection reagent only (oligofectamine; third lane from right), or left untreated (no transfectin; leftmost lane).

FIG. 6 (left panel) shows testing of compounds that inhibit validated targets (e.g., NP, HRH1, c-Yes) for anti-HIV activity and toxicity. As elsewhere herein, p24 ELISA assays were used to measure p24 production in HIV-infected cells treated with various test compounds. The SFK inhibitor PP2 resulted in substantial inhibition of HIV. The right panel shows use of XTT respiration assays to assess compound toxicity. Except for the higher concentration of doxepin, the compounds were not toxic at the concentrations used.

FIG. 7 shows that treatment of HIV-infected MT-2 cells with AZT and PP2 resulted in reduced syncytia formation. Treatments with AZT and PP2 resulted in substantially reduced syncytia formation, reflecting inhibition of HIV replication within the infected MT-2 cells.

FIG. 8A shows that the scr family kinase inhibitor PP2 (4-Amino-5-(4-chlorophenyl)-7-(t-butyl)pyrazolo[3,4-d]pyrimidine) inhibits accumulation of infectious WNV in cell culture supernatants (see EXAMPLE I, below).

FIG. 8B (shows the effect of PP2 in inhibiting accumulation of infectious virus within infected cells. SKN-MC cells were infected with WNV as above. Infectious virus in soluble lysate was measured by plaque assay on Vero cells. Samples were collected 4 hours post addition of fresh PP2 (gray bars) and 24 hours post addition of fresh PP2 (black bars).

FIG. 8C shows that levels of WNV RNA within infected SKN-MC cells did not change with PP2 addition. WNV RNA quantities were normalized to β-actin values.

FIG. 8D shows that inhibition of Src-family kinase (SFK) activity prevented maturation of the WNV E-protein (reflected by the presence of endo H-resistant E-protein). As shown elsewhere herein, inhibition of SFK activity resulted in the inhibition of the production of infectious WNV, and without limitation to any particular theory, transit of the E-protein to post-ER compartments of the secretory pathway (reflected by the presence of endo H-resistant protein) is impaired, whereby inhibition of SFK activity likely results in inhibition of the production of infectious virus.

FIG. 9A shows reduction of c-yes mRNA in transfected Huh7 cells in response to c-yes specific siRNA. 1 μg total cellular RNA was used for quantitative RT-PCR with c-yes specific primers.

FIG. 9B shows WNV in siRNA treated cultures. Culture supernatant from Huh7 cells transfected with c-yes specific siRNAs (above) were harvested 24 hpi and virus measured by plaque assay on Vero cells.

FIG. 10 shows K-means clusters corresponding to 238 gene with changes at any one time point post-WNV infection.

FIG. 11 shows a classification by function of genes up-regulated at 15 h post-infection (hpi) in West Nile Virus cells (strain NY1999).

FIGS. 12A and 12B show plots of the observed relative gene expression, for exemplary up-regulated genes (c-Yes, “A”; ribosomal protein S6 kinase, “B”; Rho GTPase activating protein 5, “C”; and CD46, “D”); that is, gene expression that was upregulated at 15 hpi according to the spotted array experiment described herein was confirmed by this RT-PCR analysis, at various times (hours) post-infection with Japanese encephalitis virus (JEV; filled diamond symbols), West Nile virus (WNV; filled square symbols) and mock infection (light triangles). Expression values are given relative to mock controls.

FIG. 13 shows the effect of tyrosine kinase inhibitors on WNV (NY1999) infection in a human hepatocellular carcinoma-derived cell line (Huh7 cells).

FIG. 14 shows the effect of src family tyrosine kinase (SFK) inhibitors on WNV (NY1999) infection in SKN-MC neuroblastoma cells.

FIG. 15 shows the amount of intracellular infectious WNV after SFK inhibitor treatment in SKN-MC cells.

FIG. 16 shows the amount of intracellular WNV RNA following PP2 treatment in SKN-MC cells.

FIG. 17 shows that C-yes-specific siRNA inhibits WNV replication in Huh7 cells.

### DETAILED DESCRIPTION OF THE INVENTION

I. Identification of HIV-Regulated Genes and Pathways, Validation of Same as Therapeutic Targets, and Provision of Therapeutic Modulators; and

II. Identification of flavivirus-Regulated, and flavivirus-Related Virus-Regulated Genes and Pathways, Validation of same as Therapeutic Targets, and Provision of Therapeutic Modulators

The present invention uses nucleic acid microarrays and gene expression profiling, along with antisense oligonucleotide, RNA interference (siRNA) methods, antibody, and small molecule modulators to identify and validate therapeutically useful gene targets that are regulated upon viral (e.g., retroviruses, human immunodeficiency virus (HIV), flaviviruses (e.g., West Nile virus (WNV), Japanese encephalitis virus (JEV), St. Louis encephalitis (SLE), yellow fever virus (YFV) and Dengue fever virus (DEN)), and flavivirus-related viruses (e.g., hepatitis C virus (HCV) that is closely related to flaviviruses)) infection, replication, maturation or progression.

The present invention provides new classes of drugs to combat, e.g., human immunodeficiency virus (HIV) infection through the identification of novel target cellular genes essential for HIV replication in human cells. Cellular drug targets were identified through gene expression profiling of viral-infected cells, and validated using target-specific gene silencing techniques with, e.g., HIV replication assays as the readout.

Recent advances in microarray technology have made possible the analysis of global gene expression patterns in cells in response to viral infection, including HIV infection. However, many of these studies utilized gene expression profiling to examine the consequences of expressing only certain HIV proteins (e.g., Tat and Nef), individually in target cells, and are not as relevant for evaluating the more complex consequences of a dynamic HIV infection. Significantly, a comprehensive analysis utilizing cell lines to examine the effects of retroviruses (e.g., HIV), flaviviruses and flavivirus-related viruses on cellular gene expression has yet to be performed. For example, only two studies published to date have analyzed acute infection by replication competent HIV (Geiss, G. K. et al., Virology 266:8-16, 2000; and Corbeil, J. et al., Genome Res 11:1198-204, 2001). Both studies utilized the CEM T cell line and a single strain of HIV, HIV_{LAI}. The study by Geiss et al (supra) analyzed only 2 time points at 48 and 72 hours post infection and the potential number of genes of interest was constrained by the use of a gene array representing only 1500 genes (a cDNA microarray). The study by Corbeil et al (supra) was more extensive, examining 6800 genes at 8 time points over a 72-hour period.

The present invention uses high-throughput gene expression profiling on an extensive platform, and gene silencing methods to identify and provide a plurality of ‘validated’ virally-induced cellular gene sequences and pathways useful as targets for modulation of viral-mediated effects and phenotype associated with HIV and other viruses. Validated gene targets correspond to those viral (e.g., retroviral, flavivirus or flavivirus-related viruses; e.g., HIV, WNV, JEV, SLE, YFV, DEN and HCV)-induced gene sequences the expression of which is required, at least to some extent, for such viral infection, replication, maturation or progression, or for such viral-mediated cellular effects, conditions, diseases and phenotypes. Inventive modulators of validated targets are agents that act by inhibiting the expression of such validated virally-induced cellular genes, by reducing or preventing the expression of mRNA from such validated virally-induced genes, or by reducing the biological activity of corresponding virally-induced cellular gene products. Inventive modulators of virally-induced cellular gene expression include, but are not limited to antisense molecules, siRNA agents, ribozymes, antibodies or antibody fragments, proteins or polypeptides as well as small molecules.

Applicants have previously used gene expression profiling, and gene silencing methods involving a KSHV-DMVEC model to identify and provide a plurality of ‘validated’ KSHV (Kaposi's Sarcoma)-induced cellular gene sequences and pathways useful as targets for modulation of KSHV-mediated effects and phenotype associated with KSHV (see Moses, A. V. et al., Ann. N.Y. Acad. Sci 975:1-12, 2002, incorporated herein by reference).

Such nucleic acid microarray techniques were used herein, in combination with HIV-infected myeloid (e.g., THP-1) and T cell (e.g., MT-2) lines, to identify and validate cellular genes and pathways useful in modulating virus during the complete replication cycle of Human immunodeficiency virus (HIV-1; e.g., the dual-tropic (X4/R5) isolate 89.6). The present EXAMPLES 1-5 (below) show that modulators of the expression of particular novel validated HIV-induced cellular gene targets are suitable agents for treating HIV and HIV-related cancer and hyperplastic/neoplastic conditions.

Likewise, such nucleic acid microarray techniques were used herein, in combination with flavivirus-infected human hepatocellular carcinoma-derived cell line (Huh7 cells) and SKN-MC neuroblastoma cells, to identify and validate cellular genes and pathways useful in modulating flavivirus maturation and progression. The present EXAMPLES 6-8 (below) show that modulators of the expression of particular novel validated flavivirus-induced cellular gene targets are suitable agents for treating flavivirus and flavivirus-related conditions.

Definitions

The term “HIV” refers to the human immunodeficiency virus.

The term “89.6” or “HIV 89.6” refers to isolate 89.6 of HIV.

The term “siRNA” or “RNAi” refers to small interfering RNA as is known in the art (see e.g.: U.S. Pat. No. 6,506,559; Milhavet et al., Pharmacological Reviews 55:629-648, 2003; and Gitlin et al., J. Virol. 77:7159-7165, 2003; incorporated herein by reference).

The term “MT-2 cells” refers cells of the art-recognized human T cell leukemia cell line MT-2.

The term “THP-1 cells” refers cells of the art-recognized human myeloid (monocyte/macrophage) cell line derived from an acute monocytic leukemia.

“MAGI cells” are Hela CD4 cells stably transfected with the β-galactosidase gene under the control of the HIV LTR. When MAGI cells are productively infected with HIV, β-galactosidase expression is induced by tat-transactivation and the number of blue cells revealed by staining is a measure of virus titer.

The term “PP2” (see formula below) refers to 4-Amino-5-(4-chlorophenyl)-7-(t-butyl)pyrazolo[3,4-d]pyrimidine (e.g., Calbiochem; catalog no. 529573), and physiologically acceptable salts thereof:

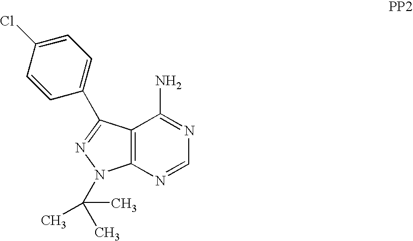

The term “PP3” refers to 4-amino-7-phenylpyrazol[3,4-D]pyrimidine (PP3) (Calbiochem), a negative control for PP2, and salts thereof:

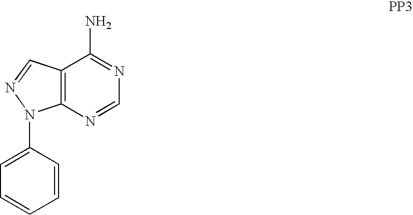

The term “SU6656” refers to (2-oxo-3-(4,5,6,7-tetrahydro-1H-indol-2-ylmethylene)-2,3-dihydro-1H-indole-5-sulfonic acid dimethylamide) (see Blake et al., Mol. Cell. Biol. 20:9018-9027, December 2000), and physiologically acceptable salts thereof.

The term “SU6657” refers to (2-oxo-3-(4,5,6,7-tetrahydro-1H-indol-2-ylmethylene)-2,3-dihydro-1H-indole-5-sulfonic acid amide) (Id), and salts thereof.

The term “PP1” refers to 4-Amino-5-(4-methylphenyl)-7-(t-butyl)pyrazolo[3,4-d]pyrimidine (e.g., from A.G. Scientific, Inc.)

The term “HMG20B” is used in reference to the gene, cDNA, mRNA or corresponding protein sequences of homo sapiens high-mobility group 20B (accession number NM_{—}006339, and known variants) (see SEQ ID NOS:1 and 2).

The term “HRH1” is used in reference to the gene, cDNA, mRNA or corresponding protein sequences of homo sapiens histamine receptor H1 (accession numbers NM_{—}00861 and BC060802, and known variants of both of these) (see SEQ ID NOS:3, 4 and 5).

The term “NP” is used in reference to the gene, cDNA, mRNA or corresponding protein sequences of homo sapiens nucleoside phosphorylase (accession number NM_{—}000270, and known variants) (see SEQ ID NOS:6 and 7).

The term “YES” or “c-YES” or “YES1” is used in reference to the gene, cDNA, mRNA or corresponding protein sequences of homo sapiens v-yes-1 Yamaguchi sarcoma viral oncogene homolog 1 (accession number NM_{—}005433, and known variants) (see SEQ ID NOS:8 and 9).

The phrase “HIV-mediated cellular effects, conditions and diseases” or “HIV-related (or mediated) conditions or diseases” refers to those illnesses and conditions included in, but not necessarily limited to the CDC 1993 AIDS surveillance case definition, as follows: Bacillary angiomatosis; Candidiasis of bronchi, trachea, or lungs; Candidiasis, esophageal; Candidiasis, oropharyngeal (thrush); Candidiasis, vulvovaginal; persistent, frequent, or poorly responsive to therapy; Cervical dysplasia (moderate or severe)/cervical carcinoma in situ; Cervical cancer, invasive *; Coccidioidomycosis, disseminated or extrapulmonary; Constitutional symptoms, such as fever (38.5 C) or diarrhea lasting greater than 1 month; Cryptococcosis, extrapulmonary; Cryptosporidiosis, chronic intestinal (greater than 1 month's duration); Cytomegalovirus disease (other than liver, spleen, or nodes); Cytomegalovirus retinitis (with loss of vision); Encephalopathy, HIV-related; Herpes simplex: chronic ulcer(s) (greater than 1 month's duration); or bronchitis, pneumonitis, or esophagitis; Hairy leukoplakia, oral; Herpes zoster (shingles), involving at least two distinct episodes or more than one dermatome; Histoplasmosis, disseminated or extrapulmonary; Idiopathic thrombocytopenic purpura; Isosporiasis, chronic intestinal (greater than 1 month's duration); Kaposi's sarcoma; Listeriosis; Lymphoma, Burkitt's (or equivalent term); Lymphoma, immunoblastic (or equivalent term); Lymphoma, primary, of brain; Mycobacterium avium complex or M. kansasii, disseminated or extrapulmonary; Mycobacterium tuberculosis, any site (pulmonary or extrapulmonary); Mycobacterium, other species or unidentified species, disseminated or extrapulmonary; Peripheral neuropathy; Pelvic inflammatory disease, particularly if complicated by tubo-ovarian abscess; Pneumocystis carinii pneumonia; Pneumonia, recurrent; Progressive multifocal leukoencephalopathy; Salmonella septicemia, recurrent; Toxoplasmosis of brain; and Wasting syndrome due to HIV. The underlying premise for the use of these phrases in the present inventive context is that the CD4+ T-lymphocyte is the primary target for HIV infection because of the affinity of the virus for the CD4 surface marker. The CD4+ T-lymphocyte coordinates a number of important immunologic functions, and a loss of these functions results in progressive impairment of the immune response. Studies of the natural history of HIV infection have documented a wide spectrum of disease manifestations, ranging from asymptomatic infection to life-threatening conditions characterized by severe immunodeficiency, serious opportunistic infections, and cancers. Other studies have shown a strong association between the development of life-threatening opportunistic illnesses and the absolute number (per microliter of blood) or percentage of CD4+ T-lymphocytes. As the number of CD4+ T-lymphocytes decreases, the risk and severity of opportunistic illnesses increase. Accordingly, treatment of HIV infection and/or replication address many related conditions and illnesses according to the present invention.

The term “WNV” refers to West Nile virus, a flavivirus.

The term “JEV: refers to Japanese encephalitis virus, a flavivirus.

The term “SLE” refers to St. Louis encephalitis, a flavivirus.

The term “DEN” refers to Dengue, a flavivirus.

The term “Huh7” refers to the art-recognized human hepatocellular carcinoma derived cell line.

The term “SKN-MC” refers to art-recognized neuroblastoma cells.

The term “SU6656” refers to (2-oxo-3-(4,5,6,7-tetrahydro-1H-indol-2-ylmethylene)-2,3-dihydro-1H-indole-5-sulfonic acid dimethylamide) (see Blake et al., Mol. Cell. Biol. 20:9018-9027, December 2000).

Myeloid cell line model system for in vivo HIV-related effects. Inventive HIV-related therapeutic targets were identified by the use of the monocyte/macrophage cell line “THP-1”. This is a human myeloid cell line derived from an acute monocytic leukemia. The cells grow in suspension and exhibit many features of monocytes, including but not limited to the ability to differentiate into macrophage-like cells following phorbol ester treatment. They express CD4 and HIV co-receptors, and are susceptible to HIV infection. THP-1 were chosen for this study because they more closely represent native monocyte/macrophages than other available human myeloid cell lines (e.g., HL60, U937, KG-1 or HEL cells) (for review see Auwerx, J. Experimentia 47:22-31, 1991, entitled “The human leukemia cell line, THP-1: a multi-facetted model for the study of monocyte-macrophage differentiation”). THP-1 cells are available through the ATCC.

Additionally, the T cell line “MT-2” was employed in the present model system. This is a human T cell line leukemia cell line. The cells grow in suspension and are very susceptible to acute infection with HIV. The cells can be efficiently loaded with antisense oligonucleotides. In addition, they have been shown by other investigators to provide a sensitive and reproducible system to test antiviral agents (see, e.g., Haertle et al, J. Biol. Chem. 263:5870-5875, 1988). MT-2 cells are available through the NIH AIDS Research and Reference Reagent Program.

Finally, the HIV-1 strain used in the model system was the 89.6 strain. This is a dual tropic (X4/R5) HIV strain, meaning that it can infect cells utilizing CD4 and either the CXCR4 or the CCR5 co-receptor. Thus, both T cells (e.g., MT-2) and macrophages (e.g., THP-1) are susceptible to infection by the same virus strain. HIV-1 89.6 was originally provided by the investigator who isolated and characterized it, Dr Ronald Collman (Collman et al, J. Virology 66:7517, 1992). Applicant's expanded the virus by culture in PBMC, and concentrated it for use in the inventive system as described in EXAMPLE 2, herein below.

Identification of HIV-induced cellular genes using microarrays. Cellular genes involved in HIV replication cycle were identified by using DNA microarrays to examine the differential gene expression profiles of THP1 monocytes before and after HIV-infection. Such microarray technology is well known in the art (see, e.g., Moses et al., J. Virol. 76:8383-8399, 2002; WO 02/10339 A2, published 7 Feb. 2002; Salunga et al., In M. Schena (ed.), DNA microarrays, A practical approach; Oxford Press, Oxford, United Kingdom, 1999; and see Simmen et al., Proc. Natl. Acad. Sci. USA 98:7140-7145, 2001; all of which are incorporated by reference herein), and can be performed using commercially available arrays (e.g., Affymetrix U133A, U133B and U95A GeneChip® arrays) (Affymetrix, Santa Clara, Calif.). The Human Genome U133 (HG-U133) set, consists of two GeneChip® arrays, and contains almost 45,000 probe sets representing more than 39,000 transcripts derived from approximately 33,000 well-substantiated human genes (Affymetrix technical information). The set design uses sequences selected from GenBank®, dbEST, and RefSeq (Id).

Specifically, as described in detail under EXAMPLE 2 herein, nucleic acid microarray technology was used for gene expression profiling of HIV-infected (synchronized) THP1 cells, relative to non-infected control cells, to identify cellular genes whose expression is regulated by HIV. Each of the TPH1 cell infected/uninfected sample comparisons resulted in multiple probe sets with increased expression, with most showing increased expression in duplicate infections. Approximately 20 genes with increased transcription in at least two adjacent time points across two infections were selected (EXAMPLE 2) for validation as described in EXAMPLE 3, herein below.

Likewise, cellular genes involved in flavivirus (e.g., WNV, JEV, YFV, DEN), and flavivirus-related virus (e.g., HCV) replication cycles were identified by using such DNA microarrays to examine the differential gene expression profiles of Huh7 cells before and after virus-infection.

Validation of therapeutic targets by gene silencing using gene-specific PMO antisense compounds. Particular virus-regulated or virus-induced gene sequences were identified as validated therapeutic targets by specific gene silencing using PMO (phosphorodiamidate morpholino Oligomers) antisense oligonucleotide inhibition in combination with measuring the effects of such gene silencing using, for example (in the case of HIV), MT-2-, or THP1-HIV replication assays (EXAMPLE 3, below). Silencing of particular target genes precluded progression of HIV replication, as measured by decreased production of HIV-1 gag protein p24, thus validating such genes as therapeutic intervention targets.

Additionally, particular virus-regulated or virus-induced gene sequences were identified as validated therapeutic targets by specific gene silencing using siRNA methods, and, in particular cases, use of small molecule modulators. Therapeutic utility. According to the present invention, siRNA, and PMO-mediated gene silencing using, for example, the THP1 and MT-2 system with HIV 89.6 (or HIV MN) not only provide validation of therapeutically-significant targets, but also provide gene-specific modulators of HIV-induced cellular gene expression that have therapeutic utility. PMOs, for example, (see, e.g., Summerton, et al., Antisense Nucleic Acid Drug Dev. 7:63-70, 1997; and Summerton & Weller, Antisense Nucleic Acid Drug Dev. 7:187-95, 1997) represent a class of art-recognized antisense drugs for treating various diseases, including cancer. For example, Arora et al. (J. Pharmaceutical Sciences 91:1009-1018, 2002) demonstrated that oral administration of c-myc-specific and CYP3A2-specific PMOs inhibited c-myc and CYP3A2 gene expression, respectively, in rat liver by an antisense mechanism of action. Likewise, Devi G. R. (Current Opinion in Molecular Therapeutics 4:138-148, 2002) discusses treatment of prostate cancer with various PMO therapeutic agents).

Likewise, siRNA” or “RNAi” agents are emerging as a new class of art-recognized drugs (see e.g.: U.S. Pat. No. 6,506,559; Milhavet et al., Pharmacological Reviews 55:629-648, 2003; and Gitlin et al., J. Virol. 77:7159-7165, 2003; incorporated herein by reference).

Accordingly, the present invention provides therapeutic compositions, and methods for modulation of viral (e.g., HIV, WNV, JEV, SLE, YFV, DEN and HCV) infection and/or replication, comprising inhibition of virally-induced cellular gene expression (e.g., inhibition of the expression of validated virally-induced genes, reducing or preventing the expression of mRNA from validated virally-induced genes, or reducing the biological activity of corresponding virally-induced cellular gene products).

Additional embodiments provide screening assays for compounds useful to modulate viral (e.g., HIV, WNV, JEV, SLE, YFV, DEN and HCV) infection, replication, maturation or progression.

Further embodiments provide diagnostic or prognostic assays for viral (e.g., HIV, WNV, JEV, SLE, YFV, DEN and HCV) infection, replication, maturation or progression.

Preferred Inventive Modulators, Compositions, Utilities and Expression Vectors

Modulators of virally-induced gene expression. Particular embodiments provide modulators of virus (e.g., HIV, WNV, JEV, SLE, YFV, DEN and HCV)-induced cellular gene expression. Preferably, inventive modulators are directed to one or more validated virus-induced cellular gene targets, the expression of which is required, at least to some extent, for virus-mediated effects on cellular proliferation and/or phenotype.

Inventive modulators include, but are not limited to, antisense molecules, siRNA, ribozymes, antibodies or antibody fragments, proteins or polypeptides as well as small molecules. Particular virus-induced gene expression modulators, such as gene-specific antisense, siRNA, and ribozyme molecules, small molecules, and antibodies and epitope-binding fragments thereof, are inhibitors of virus-induced gene expression, or of the biological activity of proteins encoded thereby.

Preferably, inventive antisense molecules are oligonucleotides of about 10 to 35 nucleotides in length that are targeted to a nucleic acid molecule corresponding to a viral-induced gene sequence, wherein the antisense molecule inhibits the expression of at least one viral-induced gene sequence. Antisense compounds useful to practice the invention include oligonucleotides containing art-recognized modified backbones or non-natural internucleoside linkages, modified sugar moieties, or modified nucleobases.

Preferred antisense molecules or the complements thereof comprise at least 10, at least 15, at least 17, at least 20, at lease 22, or at least 25, and preferably less than about 35 consecutive complementary nucleotides of, or hybridize under stringent or highly stringent conditions to at least one of the nucleic acid sequences from the group consisting of: SEQ ID NO:1 (cDNA/mRNA for HMG20B, homo sapiens high-mobility group 20B, accession number NM-006339, and known variants); SEQ ID NO:3 and SEQ ID NO:5 (cDNA/mRNA for HRH1, homo sapiens histamine receptor H1, accession numbers NM_{—}00861 and BC060802, and known variants of both of these); SEQ ID NO:6 (cDNA/mRNA for NP, homo sapiens nucleoside phosphorylase, accession number NM_{—}000270, and known variants); and SEQ ID NO:8 (cDNA/mRNA for YES1 (c-Yes), homo sapiens v-yes-1 Yamaguchi sarcoma viral oncogene homolog 1, accession number NM_{—}005433, and known variants); or to the complements thereof. Preferably, such antisense molecules are PMO (phosphorodiamidate morpholino Oligomers) antisense molecules.

Thus, the present invention includes nucleic acids that hybridize under stringent hybridization conditions, as defined below, to all or a portion of the validated HIV-induced cellular gene sequences represented by the cDNA sequences of SEQ ID NOS:1, 3, 5, 6 and 8, or the complements thereof. The hybridizing portion of the hybridizing nucleic acids is typically at least 10, at least 15, at least 17, at least 20, at least 22, at least 25, at least 30 or at least 35 nucleotides in length. Preferably, the hybridizing portion of the hybridizing nucleic acid is at least 80%, at least 90%, at least 95%, at least 98%, or at least 99% identical to the sequence of a portion or all of the cDNA sequences of SEQ ID NOs:1, 3, 5, 6 and 8, or to the complements thereof.

Hybridizing nucleic acids of the type described herein can be used, for example, as an inventive therapeutic modulator of virus-induced gene expression, a cloning probe, a primer is (e.g., a PCR primer), or a diagnostic and/or prognostic probe or primer. Preferably, hybridization of the oligonucleotide probe to a nucleic acid sample is performed under stringent conditions. Nucleic acid duplex or hybrid stability is expressed as the melting temperature or Tm, which is the temperature at which a probe dissociates from a target DNA. This melting temperature is used to define the required stringency conditions.

For sequences that are related and substantially identical to the probe, rather than identical, it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch.

Stringent conditions, as defined herein, involve hybridizing at 68° C. in 5×SSC/5× Denhardt's solution/1.0% SDS, and washing in 0.2×SSC/0.1% SDS at room temperature, or involve the art-recognized equivalent thereof. Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y; and Ausubel et al. (eds.), 1995, Current Protocols in Molecular Biology, (John Wiley & Sons, N.Y.) at Unit 2.10.

Antisense molecules preferably comprise at least 17 or at least 20, or at least 25, and preferably less than about 35 consecutive complementary nucleotides of, or hybridize under stringent conditions to at least one of the nucleic acid sequences from the group consisting of SEQ ID NOS:1, 3, 5, 6 and 8, and complements thereof. Preferably, such antisense molecules are PMO antisense molecules. Preferred representative antisense molecules are provided herein as:

| | | |
|---|---|---|
| SEQ ID NO:10 | (HMG20) | 5′-cgcccagcatcttggtgatctcggg-3′; |
| SEQ ID NO:11 | (HRH1) | 5′-gcgaaagagcagccgccagttatgg-3′; |
| SEQ ID NO:12 | (NP) | 5′-cttcataggtgtatccgttctccat-3′; |
| SEQ ID NO:13 | (c-YES) | 5′-tttctttacttttaatgcagcccat-3′; |
| and | | |
| SEQ ID NO:14 | (ARF1) | 5′-atgcttgtggacaggtggaaggaca-3′. |

Preferably, these antisense molecules are PMO antisense molecules.

Even more preferably, representative antisense molecules are provided herein as SEQ ID NOS:10, 11, 12, and 13, and these antisense molecules are preferably PMO antisense molecules.

The invention further provides a ribozyme capable of specifically cleaving at least one RNA specific to IMG20, HRH1, NP, and c-YES, and a pharmaceutical composition comprising the ribozyme.

The invention also provides small molecule modulators of virus-induced gene expression, wherein particular modulators are inhibitors capable of reducing the expression of at least one virus-induced gene, reducing or preventing the expression of mRNA from at least one virus-induced gene, or reducing the biological activity of at least one virus-induced gene product. Preferably, the virus-induced cellular gene is selected from the group consisting of HMG20, HRH1, NP, and c-YES. Preferably, the virus-induced cellular gene is c-YES.

Compositions. Further embodiments provide compositions that comprise one or more modulators of virus (e.g., HIV, WNV, JEV, SLE, YFV, DEN and HCV)-induced gene expression (or modulators of biological activity of virus-induced gene products) in a pharmaceutically acceptable carrier or diluent.

Particular embodiments provide a pharmaceutical composition for inhibiting virus-induced gene expression, comprising an antisense oligonucleotide according to the invention in a mixture with a pharmaceutically acceptable carrier or diluent.

Further provided is a composition comprising a therapeutically effective amount of an inhibitor of a virus-induced gene product (e.g., protein) in a pharmaceutically acceptable carrier. In certain embodiments, the composition comprises two or more virus-induced gene product inhibitors. Preferably, the virus-induced gene product is selected from: the nucleic acid group consisting of SEQ ID NOS:1, 3, 5, 6 and 8, and combinations thereof, corresponding to HMG20, HRH1, NP, and c-YES, and combinations thereof, respectively; or from the protein group consisting of SEQ ID NOS:2, 4, 7 and 9, and combinations thereof, corresponding to HMG20, HRH1, NP, and c-YES, respectively.

In particular composition embodiments, the virus-induced gene inhibitor is an antisense molecule, and in specific embodiments the antisense molecule or the complement thereof comprises at least 10, 15, 17, 20 or 25 consecutive nucleic acids of, or hybridizes under stringent conditions to at least one of the nucleic acid sequences from the group consisting of SEQ ID NOS:1, 3, 5, 6, and 8, and complements thereof. Preferably, such antisense molecules are PMO antisense molecules. Preferably, the antisense molecules comprises a nucleic acid sequence selected from the group consisting of SEQ ID NOS:10-13.

Methods and uses. Particular embodiments of the present invention provide methods of modulating virus (e.g., HIV, WNV, JEV, SLE, YFV, DEN and HCV)-induced gene expression or biological activity of virus-induced gene products in virus-infected cells.

The invention provides a method of inhibiting the expression of virus (e.g., HIV, WNV, JEV, SLE, YFV, DEN and HCV)-induced cellular genes in human cells or tissues comprising contacting the cells or tissues in vivo (also ex vivo, or in vitro) with an antisense compound or a ribozyme of about 10 to 35 nucleotides in length targeted to a nucleic acid molecule encoding a virus-induced gene product so that expression of the human virus-induced gene product is inhibited. Preferably, the virus-induced gene is selected from the group consisting of: HMG20B (homo sapiens high-mobility group 20B, accession number NM_{—}006339, and known variants); HRH1 (homo sapiens histamine receptor H1, accession numbers NM_{—}00861 and BC060802, and known variants of both of these); NP (homo sapiens nucleoside phosphorylase, accession number NM_{—}000270, and known variants); and YES1(homo sapiens v-yes-1 Yamaguchi sarcoma viral oncogene homolog 1, accession number NM_{—}005433, and known variants); or combinations thereof. Preferably, the antisense compounds are PMOs.

The invention additionally provides a method of modulating virus replication (or infection, maturation or progression) in cells comprising contacting the cells in vivo (also ex vivo, or in vitro) with an inventive antisense compound or ribozyme of about 10 to 35 nucleotides in length targeted to a nucleic acid molecule encoding a virus-induced gene product so that expression of the human virus-induced gene product is inhibited and, for example, viral (e.g., HIV) replication is inhibited.

The invention provides for the use of a modulator of virus-induced gene expression according to the invention to prepare a medicament for modulating virus replication (or infection, maturation or progression), virus-mediated cell proliferation and/or virus-mediated cellular phenotype.

Additional embodiments provide a method of inhibiting virus (e.g., HIV, WNV, JEV, SLE, YFV, DEN and HCV)-induced gene expression or encoded biological activity in a mammalian cell, comprising administering to the cell an inhibitor of virus-induced gene expression (or of encoded biological activity), and in a specific embodiment of the method, the inhibitor is a target gene-specific antisense molecule. Preferably, the antisense molecule is a PMO antisense molecule. Preferably, the antisense molecules comprises a nucleic acid sequence selected from the group consisting of SEQ ID NOS:10-13.

The invention also provides a method of inhibiting virus (e.g., HIV, WNV, JEV, SLE, YFV, DEN and HCV)-induced gene expression in a subject, comprising administering to said subject, in a pharmaceutically effective vehicle, an amount of an antisense oligonucleotide which is effective to specifically hybridize to all or part of a selected target nucleic acid sequence derived from said virus-induced gene. In preferred embodiments of this method, the target-specific antisense oligonucleotide is selected from the group consisting of SEQ ID NOS:10, 11, 12 and 13. Preferably the antisense oligonucleotides are PMO antisense compounds.

The invention further provides a method of treating virus (e.g., HIV, WNV, JEV, SLE, YFV, DEN and HCV)-related conditions or disease, comprising administering to a mammalian cell a modulator of virus-induced gene expression such that, for example, the neoplastic condition or virus-related disease is reduced in severity.

As discussed in the EXAMPLES herein below, additional embodiments provide screening assays for identification of compounds useful to modulate virus infection, comprising: contacting virus (e.g., HIV, WNV, JEV, SLE, YFV, DEN and HCV)-infected cells with a test agent; measuring, using a suitable assay, expression of at least one validated virus-induced cellular gene sequence; and determining whether the test agent inhibits said validated gene expression relative to control cells not contacted with the test agent, whereby agents that inhibit said validated gene expression are identified as compounds useful to modulate virus infection.

Preferably, expression of at least one validated virus-induced cellular gene sequence is expression of respective mRNA, or expression of the protein encoded thereby.

Preferably, the at least one validated virus-induced cellular gene sequence is selected from the cDNA and protein sequence group consisting of HMG20, HRH1, NP, and c-YES, and combinations thereof (i.e., consisting of SEQ ID NOS:1-9, and combinations thereof).

Preferably, agents that inhibit said validated gene expression are further tested for the ability to modulate respective virus infection and/or replication, virus-mediated cellular proliferation and/or virus-mediated cellular phenotype, conditions or diseases.

Further embodiments provide diagnostic or prognostic assays for viral (e.g., HIV, WNV, JEV, SLE, YFV, DEN and HCV) infection, replication, maturation or progression, comprising: obtaining a cell sample from a subject suspected of having virus; measuring expression of at least one validated virus-inducible cellular gene sequence; and determining whether expression of the at least one validated gene is induced relative to non-virus-infected control cells, whereby a diagnosis is, at least in part, afforded.

Preferably, the at least one validated virus-inducible cellular gene is selected from the cDNA and protein sequence group consisting of HMG20, HRH1, NP, and c-YES, and combinations thereof (i.e., consisting of SEQ ID NOS:1-9, and combinations thereof).

Preferably, measuring said expression is of two or more validated virus-inducible cellular gene sequences. Preferably, measurement of said expression is by use of high-throughput microarray methods.

Polynucleotides and expression vectors. Particular embodiments provide an isolated polynucleotide with a sequence comprising a transcriptional initiation region and a sequence encoding a virus-induced gene-specific antisense oligonucleotide at least 10, 15, 17, 20, 22 or 25 nucleotides in length, and a recombinant vector comprising this polynucleotide (e.g., expression vector). Preferably, the antisense oligonucleotide of said polynucleotide comprises a sequence selected from the group consisting of SEQ ID NOS:10-13. Preferably, the transcriptional initiation region is a strong constitutively expressed mammalian pol III- or pol II-specific promoter, or a viral promoter.

Additional and Preferred Oligonucleotide Modulators

Included within the scope of the invention are oligonucleotides capable of hybridizing with virus (e.g., HIV, WNV, JEV, SLE, YFV, DEN and HCV)-induced gene DNA or RNA, referred to herein as the ‘target’ polynucleotide. An oligonucleotide need not be 100% complementary to the target polynucleotide, as long as specific hybridization is achieved. The degree of hybridization to be achieved is that which interferes with the normal function of the target polynucleotide, be it transcription, translation, pairing with a complementary sequence, or binding with another biological component such as a protein. An antisense oligonucleotide, including a preferred PMO antisense oligonucleotide, can interfere with DNA replication and transcription, and it can interfere with RNA translocation, translation, splicing, and catalytic activity.

The invention includes within its scope any oligonucleotide of about 10 to about 35 nucleotides in length, including variations as described herein, wherein the oligonucleotide hybridizes to a virus-induced target sequence, including DNA or mRNA, such that an effect on the normal function of the polynucleotide is achieved. The oligonucleotide can be, for example, 10, 15, 17, 20, 22, 23, 25, 30 or 35 nucleotides in length. Oligonucleotides larger than 35 nucleotides are also contemplated within the scope of the present invention, and may for example, correspond in length to a complete target cDNA (i.e., mRNA) sequence, or to a significant or substantial portion thereof.

Antisense oligonucleotides. As described above, preferred antisense molecules are represented by SEQ ID NOS:10-13, and combinations thereof.

Examples of representative preferred antisense compounds useful in the invention are based on SEQ ID NOS:1, 3, 5, 6, 8 and 10-13, and include oligonucleotides containing modified backbones or non-natural internucleoside linkages. Oligonucleotides having modified backbones include those retaining a phosphorus atom in the backbone, and those that do not have a phosphorus atom in the backbone.

Preferred modified oligonucleotide backbones include phosphorothioates or phosphorodithioate, chiral phosphorothioates, phosphotriesters and alkyl phosphotriesters, aminoalkylphosphotriesters, methyl and other alkyl phosphonates including methylphosphonates, 3′-alkylene phosphonates and chiral phosphonates, phosphinates, phosphoroamidates or phosphordiamidates, including 3′-amino phosphoroamidate and aminoalkylphosphoroamidates, and phosphorodiamidate morpholino oligomers (PMOs), thiophosphoroamidates, phosphoramidothioates, thioalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

The antisense oligonucleotide may also comprise at least one modified sugar moiety selected from the group including, but not limited to arabinose, 2-fluoroarabinose, xylulose, hexose and 2′-O-methyl sugar moieties.

The antisense oligonucleotide may comprise at least one modified base moiety which is selected from the group including, but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine (see also U.S. Pat. No. 5,958,773 and patents disclosed therein).

Examples of inventive antisense oligonucleotides of length X (in nucleotides), as indicated by polynucleotide positions with reference to, e.g., SEQ ID NO:1, include those corresponding to sets of consecutively overlapping oligonucleotides of length X, where the oligonucleotides within each consecutively overlapping set (corresponding to a given X value) are defined as the finite set of Z oligonucleotides from nucleotide positions:
n to (n+(X−1));

where n=1, 2, 3, . . . (Y−(X−1));

where Y equals the length (nucleotides or base pairs) of SEQ ID NO:1 (1,232);

where X equals the common length (in nucleotides) of each oligonucleotide in the set (e.g., X=20 for a set of consecutively overlapping 20-mers); and

where the number (Z) of consecutively overlapping oligomers of length X for a given SEQ ID NO of length Y is equal to Y−(X−1). For example Z=1,232−19=1,213 for SEQ ID NO:1, where X=20.

Examples of inventive 20-mer oligonucleotides include the following set of 1,213 oligomers, indicated by polynucleotide positions with reference to SEQ ID NO:1 (HMG20B cDNA): 1-20, 2-21, 3-22, 4-23, 5-24, . . . , 1211-1230, 1212-1231 and 1213-1232.

Likewise, examples of 25-mer oligonucleotides include the following set of 1,208 oligomers, indicated by polynucleotide positions with reference to SEQ ID NO:1: 1-25, 2-26, 3-27, 4-28, 5-29 . . . 1206-1230, 1207-1231 and 1208-1232.

The present invention encompasses, for each validated target sequence (e.g., for SEQ ID NOS:1, 3, 5, 6, and 8, and the complements thereof), multiple consecutively overlapping sets of oligonucleotides or modified oligonucleotides of length X, where, e.g., X=10, 17, 20, 22, 23, 25, 30 or 35 nucleotides.

Preferred sets of such oligonucleotides or modified oligonucleotides of length X are those consecutively overlapping sets of oligomers corresponding to SEQ ID NOS:1, 3, 5, 6, and 8, and to the complements thereof. Included in these preferred sets are the preferred oligomers corresponding to SEQ ID NOS:10-13.

Representative siRNA sequence regions are disclosed herein, in view of the above algorithm in combination with the teachings on design (e.g., length, structure, composition, etc), preparation and use thereof, provided herein below under “siRNA.”

The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. Thus, the oligonucleotide may include other appended groups such as peptides (e.g., for targeting host cell receptors in vivo), or agents facilitating or modulating transport across the cell membrane (Letsinger et al., Proc. Natl. Acad. Sci. USA 86:6553-6556, 1989; Lemaitre et al., Proc. Natl. Acad. Sci. USA 84:648-652, 1987; PCT WO88/09810, published Dec. 15, 1988) or the blood-brain barrier (PCT WO89/10134, published Apr. 25, 1988), or the nuclear membrane, and may include hybridization-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a peptide, hybridization-triggered cross-linking agent, transport agent, hybridization-triggered cleavage agent, etc.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

Preferred antisense oligonucleotides in addition to those of SEQ ID NOS:10-14 are selected by routine experimentation using, for example, assays described in the present Examples. Although the inventors are not bound by a particular mechanism of action, it is believed that the antisense oligonucleotides achieve an inhibitory effect by binding to a complementary region of the target polynucleotide within the cell using Watson-Crick base pairing. Where the target polynucleotide is RNA, experimental evidence indicates that the RNA component of the hybrid is cleaved by RNase H (Giles, R. V. et al., Nuc. Acids Res. (1995) 23:954-961; U.S. Pat. No. 6,001,653). Generally, a hybrid containing 10 base pairs is of sufficient length to serve as a substrate for RNase H. However, to achieve specificity of binding, it is preferable to use an antisense molecule of at least 17 nucleotides, as a sequence of this length is likely to be unique among human genes.

Antisense approaches comprise the design of oligonucleotides (either DNA or RNA) that are complementary to the target gene sequence (e.g., mRNA). The antisense oligonucleotides bind to the complementary mRNA transcripts and prevent translation. Absolute complementarily, although preferred, is not required. A sequence “complementary” to a portion or region of the target mRNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize depends on both the degree of complementarity and the length of the antisense nucleic acid. Generally, the longer the hybridizing nucleic acid, the more base mismatches with an RNA are accommodated without compromising stable duplex (or triplex, as the case may be) formation. One skilled in the art ascertains a tolerable degree of mismatch by use of standard procedures to determine the melting point of the hybridized complex.

As disclosed in U.S. Pat. No. 5,998,383, incorporated herein by reference, the oligonucleotide is selected such that the sequence exhibits suitable energy related characteristics important for oligonucleotide duplex formation with their complementary targets, and shows a low potential for self-dimerization or self-complementation (Anazodo et al., Biochem. Biophys. Res. Commun. (1996) 229:305-309). The computer program OLIGO (Primer Analysis Software, Version 3.4), is used to determined antisense sequence melting temperature, free energy properties, and to estimate potential self-dimer formation and self-complementarity properties. The program allows the determination of a qualitative estimation of these two parameters (potential self-dimer formation and self-complementary) and provides an indication of “no potential” or “some potential” or “essentially complete potential.” Preferably, segments of validated HIV-induced gene sequences are selected that have estimates of no potential in these parameters. However, segments that have “some potential” in one of the categories nonetheless can have utility, and a balance of the parameters is routinely used in the selection.

While antisense nucleotides complementary to the coding region sequence of a mRNA are used in accordance with the invention, those complementary to the transcribed, untranslated region, or translational initiation site region are sometimes preferred. Oligonucleotides that are complementary to the 5′ end of the message, e.g., the 5′-untranslated sequence (up to and including the AUG initiation codon), frequently work most efficiently at inhibiting translation. However, sequences complementary to the 3′-untranslated sequences, or other regions of mRNAs are also effective at inhibiting translation of mRNAs (see e.g., Wagner, Nature 372:333-335, 1994). In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation.

Such experimentation can be performed routinely by transfecting or loading cells with an antisense oligonucleotide, followed by measurement of messenger RNA (mRNA) levels in the treated and control cells by reverse transcription of the mRNA and assaying of respective cDNA levels. Measuring the specificity of antisense activity by assaying and analyzing cDNA levels is an art-recognized method of validating antisense results. Routinely, RNA from treated and control cells is reverse-transcribed and the resulting cDNA populations are analyzed (Branch, A. D., T.I.B.S. (1998) 23:45-50).

According to the present invention, antisense efficacy can be alternately determined by measuring the biological effects on cell growth, phenotype or viability as is known in the art, and as shown in the present Examples. According to particular aspects of the present invention, cultures of, for example, HIV-infected TPH1 cells or MT-2 cells were loaded with inventive oligonucleotides designed to target HIV-induced gene sequences. Preferred representative antisense oligonucleotides correspond to SEQ ID NOS:10-14. The effects of such loading on HIV replication were measured. Specifically, SEQ ID NOS:10-13 caused dramatic decreases in HIV replication, as measured by decreases in HIV gag 24 protein, a hallmark of in vivo HIV-related replication.

Ribozymes. Modulators of virus (e.g., HIV, WNV, JEV, SLE, YFV, DEN and HCV)-induced gene expression may be ribozymes. A ribozyme is an RNA molecule that specifically cleaves RNA substrates, such as mRNA, resulting in specific inhibition or interference with cellular gene expression. As used herein, the term ribozymes includes RNA molecules that contain antisense sequences for specific recognition, and an RNA-cleaving enzymatic activity. The catalytic strand cleaves a specific site in a target RNA at greater than stoichiometric concentration. Preferably the ribozyme is engineered so that the cleavage recognition site is located near the 5′ end of the target mRNA (i.e., to increase efficiency and minimize the intracellular accumulation of non-functional mRNA transcripts).

A wide variety of ribozymes may be utilized within the context of the present invention, including for example, the hammerhead ribozyme (for example, as described by Forster and Symons, Cell (1987) 48:211-220; Haseloff and Gerlach, Nature (1988) 328:596-600; Walbot and Bruening, Nature (1988) 334:196; Haseloff and Gerlach, Nature (1988) 334:585); the hairpin ribozyme (for example, as described by Haseloff et al., U.S. Pat. No. 5,254,678, issued Oct. 19, 1993 and Hempel et al., European Patent Publication No. 0 360 257, published Mar. 26, 1990); and Tetrahymena ribosomal RNA-based ribozymes (see Cech et al., U.S. Pat. No. 4,987,071). The Cech-type ribozymes have an eight-base pair active site that hybridizes to a target RNA sequence whereafter cleavage of the target RNA takes place. Ribozymes of the present invention typically consist of RNA, but may also be composed of DNA, nucleic acid analogs (e.g., phosphorothioates), or chimerics thereof (e.g., DNA/RNA/RNA).

Ribozymes can be targeted to any RNA transcript and can catalytically cleave such transcripts (see, e.g., U.S. Pat. No. 5,272,262; U.S. Pat. No. 5,144,019; and U.S. Pat. Nos. 5,168,053, 5,180,818, 5,116,742 and 5,093,246 to Cech et al.). According to certain embodiments of the invention, any such virus-induced gene sequence-specific ribozyme, or a nucleic acid encoding such a ribozyme, may be delivered to a host cell to effect inhibition of virus-induced gene expression. Ribozymes and the like may therefore be delivered to the host cells by DNA encoding the ribozyme linked to a eukaryotic promoter (e.g., a strong constitutively expressed pol III- or pol II-specific promoter), or a eukaryotic viral promoter, such that upon introduction into the nucleus, the ribozyme will be directly transcribed.

Triple-helix formation. Alternatively, validated virus-induced gene expression can be reduced by targeting deoxyribonucleotide sequences complementary to the regulatory region of the target gene (e.g., respective promoter and/or enhancers) to form triple helical structures that prevent transcription of the target gene (see, e.g., Helen, Anticancer Drug Des., 6:569-84, 1991; Helene et al., Ann, N.Y. Acad. Sci., 660:27-36, 1992; and Maher, Bioassays 14:807-15, 1992).

siRNA. The invention, in particular aspects, contemplates introduction of RNA with partial or fully double-stranded character into the cell or into the extracellular environment. According to particular aspects of the present invention, inhibition is specific to the particular validated virus-induced cellular gene expression product in that a nucleotide sequence from a portion of the validated sequence is chosen to produce inhibitory RNA. This process is effective in producing inhibition (partial or complete), and is validated gene-specific. In particular embodiments, the target cell containing the validated gene may be a human cell subject to infection by the respective virus (or cell-lines derived therefrom).

In other aspects of the invention, inhibition is specific to the src family kinase expression in that a nucleotide sequence from a portion of the target src family kinase gene is chosen to produce inhibitory RNA. This process is effective in producing inhibition (partial or complete), and is kinase gene-specific. In particular embodiments, the target cell containing the target src family kinase gene may be a human cell subject to infection by HIV, flaviviruses or HCV, or transformed cells (e.g., in the case of flaviviruses, hepatocellular carcinoma cells or cell-lines derived therefrom, or neuroblastoma cells or cell-lines derived therefrom).

Methods of preparing and using siRNA are generally disclosed in U.S. Pat. No. 6,506,559, incorporated herein by reference (see also reviews by Milhavet et al., Pharmacological Reviews 55:629-648, 2003; and Gitlin et al., J. Virol. 77:7159-7165, 2003; incorporated herein by reference).

The siRNA may comprise one or more strands of polymerized ribonucleotide, and may include modifications to either the phosphate-sugar backbone or the nucleoside. For example, the phosphodiester linkages of natural RNA may be modified to include at least one of a nitrogen or sulfur heteroatom. Modifications in RNA structure may be tailored to allow specific genetic inhibition while avoiding a general panic response in some organisms which is generated by dsRNA. Likewise, bases may be modified to block the activity of adenosine deaminase. RNA may be produced enzymatically or by partial/total organic synthesis, any modified ribonucleotide can be introduced by in vitro enzymatic or organic synthesis.

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses of double-stranded material may yield more effective inhibition. Inhibition is sequence-specific in that nucleotide sequences corresponding to the duplex region of the RNA are targeted for genetic inhibition. Nucleic acid containing a nucleotide sequence identical to a portion of the validated gene sequence is preferred for inhibition. RNA sequences with insertions, deletions, and single point mutations relative to the target sequence have also been found to be effective for inhibition. Sequence identity may be optimized by alignment algorithms known in the art and calculating the percent difference between the nucleotide sequences. Alternatively, the duplex region of the RNA may be defined functionally as a nucleotide sequence that is capable of hybridizing with a portion of the target gene transcript.

RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region may be used to transcribe the RNA strand (or strands).

For siRNA (RNAi), the RNA may be directly introduced into the cell (i.e., intracellularly); or introduced extracellularly into a cavity, interstitial space, into the circulation of an organism, introduced orally, or may be introduced by bathing an organism in a solution containing RNA. Methods for oral introduction include direct mixing of RNA with food of the organism, as well as engineered approaches in which a species that is used as food is engineered to express a RNA, then fed to the organism to be affected. Physical methods of introducing nucleic acids include injection directly into the cell or extracellular injection into the organism of an RNA solution.

Inhibition of gene expression refers to the absence (or observable decrease) in the level of protein and/or mRNA product from a validated gene target (e.g., inhibition of gene expression may refer to the absence (or observable decrease) in the level of protein and/or mRNA product from a src family kinase target gene). Specificity refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, fluorescence activated cell analysis (FACS), and viral infection, replication, maturation or progression assays as described herein. For RNA-mediated inhibition in a cell line or whole organism, gene expression is conveniently assayed by use of a reporter or drug resistance gene whose protein product is easily assayed. Many such reporter genes are known in the art.

The phosphodiester linkages of natural RNA may be modified to include at least one of a nitrogen or sulfur heteroatom. Modifications in RNA structure may be tailored to allow specific genetic inhibition while avoiding a general panic response in some organisms which is generated by dsRNA. Likewise, bases may be modified to block the activity of adenosine deaminase. RNA may be produced enzymatically or by partial/total organic synthesis, any modified ribonucleotide can be introduced by in vitro enzymatic or organic synthesis.

RNA containing a nucleotide sequence identical to a portion of a particular validated gene sequence are preferred for inhibition. In particular aspects, RNA containing a nucleotide sequence identical to a portion of the src family kinase target gene are preferred for inhibition. RNA sequences with insertions, deletions, and single point mutations relative to the target sequence may be effective for inhibition. Sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of particular validated gene (e.g., src family kinase target gene) sequence is preferred. Alternatively, the duplex region of the RNA may be defined functionally as a nucleotide sequence that is capable of hybridizing with a portion of the particular validated gene transcript (e.g., 400 mM NaCl, 40 mM PIPES pH 6.4, 1 mM EDTA, 50° C. or 70° C. hybridization for 12-16 hours; followed by washing). The length of the identical nucleotide sequences may be at least 20, 25, 50, 100, 200, 300 or 400 bases.

A 100% sequence identity between the RNA and a particular validated gene sequence is not required to practice the present invention. Thus the methods have the advantage of being able to tolerate sequence variations that might be expected due to genetic mutation, strain polymorphism, or evolutionary divergence.

Particular validated gene sequence siRNA (e.g., src family kinase siRNA) may be synthesized by art-recognized methods either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands). Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus.

RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in the art (e.g., WO 97/32016; U.S. Pat. Nos. 5,593,874, 5,698,425, 5,712,135, 5,789,214, and 5,804,693; and the references cited therein). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

siRNA may be directly introduced into the cell (i.e., intracellularly); or introduced extracellularly into a cavity, interstitial space, into the circulation of an organism, introduced orally, or may be introduced by bathing an organism in a solution containing the RNA. Methods for oral introduction include direct mixing of the RNA with food of the organism, as well as engineered approaches in which a species that is used as food is engineered to express the RNA, then fed to the organism to be affected. For example, the RNA may be sprayed onto a plant or a plant may be genetically engineered to express the RNA in an amount sufficient to kill some or all of a pathogen known to infect the plant. Physical methods of introducing nucleic acids, for example, injection directly into the cell or extracellular injection into the organism, may also be used. Vascular or extravascular circulation, the blood or lymph system, and the cerebrospinal fluid are sites where the RNA may be introduced. A transgenic organism that expresses RNA from a recombinant construct may be produced by introducing the construct into a zygote, an embryonic stem cell, or another multipotent cell derived from the appropriate organism.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or other-wise increase inhibition of the target gene.

The siRNA may be used alone or as a component of a kit having at least one of the reagents necessary to carry out the in vitro or in vivo introduction of RNA to test samples or subjects. Preferred components are the dsRNA and a vehicle that promotes introduction of the dsRNA. Such a kit may also include instructions to allow a user of the kit to practice the invention.

Suitable injection mixes are constructed so animals receive an average of 0.5×10⁶ to 1.0×10⁶ molecules of RNA. For comparisons of sense, antisense, and dsRNA activities, injections are compared with equal masses of RNA (i.e., dsRNA at half the molar concentration of the single strands). Numbers of molecules injected per adult are given as rough approximations based on concentration of RNA in the injected material (estimated from ethidium bromide staining) and injection volume (estimated from visible displacement at the site of injection). A variability of several-fold in injection volume between individual animals is possible.

In particular aspects of the present invention, preferred siRNA is represented by SEQ ID NOS:15 and 16, corresponding to c-YES-specific siRNA-214 and c-YES-specific siRNA-318, respectively. SEQ ID NO:17 corresponds to the human c-YES RNA sequence.

Proteins and Polypeptides

In addition to the antisense molecules, siRNA and ribozymes disclosed herein, inventive modulators of virus (e.g., HIV, WNV, JEV, SLE, YFV, DEN and HCV)-induced gene expression also include proteins or polypeptides that are effective in either reducing validated virus-induced cellular gene expression or in decreasing one or more of the respective biological activities encoded thereby. A variety of art-recognized methods are used by the skilled artisan, through routine experimentation, to rapidly identify such modulators of virus-induced gene expression. The present invention is not limited by the following exemplary methodologies.

Inhibitors of virus-induced biological activities encompass those proteins and/or polypeptides that interfere with said biological activities. Such interference may occur through direct interaction with active domains of the proteins of validated gene targets, or indirectly through non- or un-competitive inhibition such as via binding to an allosteric site. Accordingly, available methods for identifying proteins and/or polypeptides that bind to proteins of validated gene targets may be employed to identify lead compounds that may, through the methodology disclosed herein, be characterized for their inhibitory activity.

Methods for detecting and analyzing protein-protein interactions are described in the art, and are thus available to skilled artisans (reviewed in Phizicky, E. M. et al., Microbiological Reviews (1995) 59:94-123 incorporated herein by reference. Such methods include, but are not limited to physical methods such as, e.g., protein affinity chromatography, affinity blotting, immunoprecipitation and cross-linking as well as library-based methods such as, e.g., protein probing, phage display and two-hybrid screening. Other methods that may be employed to identify protein-protein interactions include genetic methods such as use of extragenic suppressors, synthetic lethal effects and unlinked noncomplementation. Exemplary methods are described in further detail below.

Inventive inhibitors of proteins of validated gene targets (validated proteins) may be identified through biological screening assays that rely on the direct interaction between the a validated protein (e.g., SEQ ID NOS:2, 4, 7, and 9) and a panel or library of potential inhibitor proteins. Biological screening methodologies, including the various “n-hybrid technologies,” are described in, for example, Vidal, M. et al., Nucl. Acids Res. (1999) 27(4):919-929; Frederickson, R. M., Curr. Opin. Biotechnol. (1998) 9(1):90-6; Brachmann, R. K. et al., Curr. Opin. Biotechnol. (1997) 8(5):561-568; and White, M. A., Proc. Natl. Acad. Sci. U.S.A. (1996) 93:10001-10003 each of which is incorporated herein by reference.

The two-hybrid screening methodology may be employed to search new or existing target cDNA libraries for inhibitory proteins. The two-hybrid system is a genetic method that detects protein-protein interactions by virtue of increases in transcription of reporter genes. The system relies on the fact that site-specific transcriptional activators have a DNA-binding domain and a transcriptional activation domain. The DNA-binding domain targets the activation domain to the specific genes to be expressed. Because of the modular nature of transcriptional activators, the DNA-binding domain may be severed from the otherwise covalently linked transcriptional activation domain without loss of activity of either domain. Furthermore, these two domains may be brought into juxtaposition by protein-protein contacts between two proteins unrelated to the transcriptional machinery. Thus, two hybrids are constructed to create a functional system. The first hybrid, i.e., the bait, consists of a transcriptional activator DNA-binding domain fused to a protein of interest (e.g., SEQ ID NOS:2, 4, 7, and 9, or fragments thereof). The second hybrid, the target, is created by the fusion of a transcriptional activation domain with a library of proteins or polypeptides. Interaction between the bait protein and a member of the target library results in the juxtaposition of the DNA-binding domain and the transcriptional activation domain and the consequent up-regulation of reporter gene expression.

A variety of two-hybrid based systems are available to the skilled artisan that most commonly employ either the yeast Gal4 or E. coli LexA DNA-binding domain (BD) and the yeast Gal4 or herpes simplex virus VP16 transcriptional activation domain. Chien, C.-T. et al., Proc. Natl. Acad. Sci. U.S.A. (1991) 88:9578-9582; Dalton, S. et al., Cell (1992) 68:597-612; Durfee, T. K. et al., Genes Dev. (1993) 7:555-569; Vojtek, A. B. et al., Cell (1993) 74:205-214; and Zervos, A. S. et al., Cell (1993) 72:223-232. Commonly used reporter genes include the E. coli lacZ gene as well as selectable yeast genes such as HIS3 and LEU2. Fields, S. et al., Nature (London) (1989) 340:245-246; Durfee, T. K., supra; and Zervos, A. S., supra. A wide variety of activation domain libraries is readily available in the art such that the screening for interacting proteins may be performed through routine experimentation.

Suitable bait proteins for the identification of inhibitors of validated proteins are designed based on the validated sequences presented herein as SEQ ID NO:2, 4, 7 and 9. Such bait proteins include either the full-length validated protein, or fragments thereof.

Plasmid vectors, such as, e.g., pBTM116 and pAS2-1, for preparing validated protein bait constructs and target libraries are readily available to the artisan and may be obtained from such commercial sources as, e.g., Clontech (Palo Alto, Calif.), Invitrogen (Carlsbad, Calif.) and Stratagene (La Jolla, Calif.). These plasmid vectors permit the in-frame fusion of cDNAs with the DNA-binding domains as LexA or Gal4BD, respectively.

Validated protein inhibitors of the present invention may alternatively be identified through one of the physical or biochemical methods available in the art for detecting protein-protein interactions.

For example, affinity chromatography may be used to identify potential inhibitors of validated proteins, by virtue of specific retention of such potential inhibitors to validated proteins, or to fragments thereof covalently or non-covalently coupled to a solid matrix such as, e.g., Sepharose beads. The preparation of protein affinity columns is described in, for example, Beeckmans, S. et al., Eur. J. Biochem. (1981) 117:527-535 and Formosa, T. et al., Methods Enzymol. (1991) 208:24-45. Cell lysates containing the full complement of cellular proteins may be passed through a validated protein affinity column. Proteins having a high affinity for the validated protein will be specifically retained under low-salt conditions while the majority of cellular proteins will pass through the column. Such high affinity proteins may be eluted from the immobilized validated protein, or fragment thereof under conditions of high-salt, with chaotropic solvents or with sodium dodecyl sulfate (SDS). In some embodiments, it may be preferred to radiolabel the cells prior to preparing the lysate as an aid in identifying the validated protein-specific binding proteins. Methods for radiolabeling mammalian cells are well known in the art and are provided, e.g., in Sopta, M. et al., J. Biol. Chem. (1985) 260:10353-10360.

Suitable validated proteins for affinity chromatography may be fused to a protein or polypeptide to permit rapid purification on an appropriate affinity resin. For example, a validated protein cDNA may be fused to the coding region for glutathione S-transferase (GST) which facilitates the adsorption of fusion proteins to glutathione-agarose columns. Smith et al., Gene (1988) 67:31-40. Alternatively, fusion proteins may include protein A, which can be purified on columns bearing immunoglobulin G; oligohistidine-containing peptides, which can be purified on columns bearing Ni²⁺; the maltose-binding protein, which can be purified on resins containing amylose; and dihydrofolate reductase, which can be purified on methotrexate columns. One such tag suitable for the preparation of validate protein fusion proteins is the epitope for the influenza virus hemagglutinin (HA) against which monoclonal antibodies are readily available and from which antibodies an affinity column may be prepared.

Proteins that are specifically retained on a validated protein affinity column may be identified after subjecting to SDS polyacrylamide gel electrophoresis (SDS-PAGE). Thus, where cells are radiolabeled prior to the preparation of cell lysates and passage through the validated protein affinity column, proteins having high affinity for the particular validate protein may be detected by autoradiography. The identity of particular validated protein-specific binding proteins may be determined by protein sequencing techniques that are readily available to the skilled artisan, such as those described by Mathews, C. K. et al., Biochemistry, The Benjamin/Cummings Publishing Company, Inc. pp. 166-170 (1990).

Antibodies or Antibody Fragments

Inhibitors of virus (e.g., HIV, WNV, JEV, SLE, YFV, DEN and HCV)-induced gene expression of the present invention include antibodies and/or antibody fragments that are effective in reducing virus-induced gene expression and/or reducing the biological activity encoded thereby. Suitable antibodies may be monoclonal, polyclonal or humanized monoclonal antibodies. Antibodies may be derived by conventional hybridoma based methodology, from antisera isolated from validated protein inoculated animals or through recombinant DNA technology. Alternatively, inventive antibodies or antibody fragments may be identified in vitro by use of one or more of the readily available phage display libraries. Exemplary methods are disclosed herein.

In one embodiment of the present invention, validated protein inhibitors are monoclonal antibodies that may be produced as follows. Validated proteins (e.g., SEQ ID NOS:2, 4, 7 and 9) may be produced, for example, by expression of the respective cDNAs (e.g., SEQ ID NOS:1, 3, 5, 6, and 8, respectively) in a baculovirus based system. By this method, validated protein cDNAs (SEQ ID NOS:1, 3, 5, 6, and 8) or epitope-bearing fragments thereof are ligated into a suitable plasmid vector that is subsequently used to transfect Sf9 cells to facilitate protein production. In addition, it may be advantageous to incorporate an epitope tag or other moiety to facilitate affinity purification of the validated protein. Clones of Sf9 cells expressing a particular validated protein are identified, e.g., by enzyme-linked immunosorbant assay (ELISA), lysates are prepared and the validated protein purified by affinity chromatography. The purified validated protein is, for example, injected intraperitoneally, into BALB/c mice to induce antibody production. It may be advantageous to add an adjuvant, such as Freund's adjuvant, to increase the resulting immune response.

Serum is tested for the production of specific antibodies, and spleen cells from animals having a positive specific antibody titer are used for cell fusions with myeloma cells to generate hybridoma clones. Supernatants derived from hybridoma clones are tested for the presence of monoclonal antibodies having specificity against a particular validated protein (e.g., SEQ ID NO:2, 4, 7, and 9, or fragments thereof). For a general description of monoclonal antibody methodology, See, e.g., Harlow and Lane, Antibodies: A Laboratory Manual, Cold Spring Harbor Laboratory (1988).

In addition to the baculovirus expression system, other suitable bacterial or yeast expression systems may be employed for the expression of a particular validated protein or polypeptides thereof. As with the baculovirus system, it may be advantageous to utilize one of the commercially available affinity tags to facilitate purification prior to inoculation of the animals. Thus, the validated protein cDNA or fragment thereof may be isolated by, e.g., agarose gel purification and ligated in frame with a suitable tag protein such as 6-His, glutathione-S-transferase (GST) or other such readily available affinity tag. See, e.g., Molecular Biotechnology: Principles and Applications of Recombinant DNA, ASM Press pp. 160-161 (ed. Glick, B. R. and Pasternak, J. J. 1998).

In additional embodiments of the present invention, inhibitors of validated proteins are humanized anti-validated protein monoclonal antibodies. The phrase “humanized antibody” refers to an antibody derived from a non-human antibody—typically a mouse monoclonal antibody. Alternatively, a humanized antibody may be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

Because humanized antibodies are far less immunogenic in humans than the parental mouse monoclonal antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, e.g., Jones et al., Nature (1986) 321:522-525; Morrison et al., Proc. Natl. Acad. Sci., U.S.A., (1984) 81:6851-6855; Morrison and Oi, Adv. Immunol. (1988) 44:65-92; Verhoeyer et al., Science (1988) 239:1534-1536; Padlan, Molec. Immun. (1991) 28:489-498; Padlan, Molec. Immunol. (1994) 31(3):169-217; and Kettleborough, C. A. et al., Protein Eng. (1991) 4:773-83 each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. (1987) 196:901-917; Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g., via Ashwell receptors (see, e.g., U.S. Pat. Nos. 5,530,101 and 5,585,089, both incorporated herein by reference.

Humanized antibodies to a particular validated protein can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy claims, and express an exogenous immunoglobulin locus comprising one or more xenogeneic constant regions.

Using a transgenic animal described above, an immune response can be produced to a selected antigenic molecule (e.g., validated protein or fragment thereof), and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. This publication discloses monoclonal antibodies against a variety of antigenic molecules including IL-6, IL-8, TNFα, human CD4, L-selectin, gp39, and tetanus toxin. The monoclonal antibodies can be tested for the ability to inhibit or neutralize the biological activity or physiological effect of the corresponding protein. WO 96/33735 discloses that monoclonal antibodies against IL-8, derived from immune cells of transgenic mice immunized with IL-8, blocked IL-8-induced functions of neutrophils. Human monoclonal antibodies with specificity for the antigen used to immunize transgenic animals are also disclosed in WO 96/34096.

For purposes of the present invention, validated polypeptides and variants thereof are used to immunize a transgenic animal as described above. Monoclonal antibodies are made using methods known in the art, and the specificity of the antibodies is tested using isolated validated polypeptides. The suitability of the antibodies for clinical use is tested by, for example, exposing HIV-infected THP1 or MT-2 cells to the antibodies and measuring cell growth and/or phenotypic changes. According to particular aspects of the invention, inhibition of virus (e.g., HIV, WNV, JEV, SLE, YFV, DEN and HCV)-induced gene sequence expression using antisense oligonucleotides specific for validated virus-induced polynucleotides causes an inhibition of virus replication in THP1 and MT-2 cells. Human monoclonal antibodies specific for a particular validated protein, or for a variant or fragment thereof can be tested for their ability to inhibit virus replication, infection, maturation or progression. Such antibodies would be suitable for pre-clinical and clinical trials as pharmaceutical agents for preventing or controlling virus-mediated effects, conditions or diseases.

It will be appreciated that alternative validated protein inhibitor antibodies may be readily obtained by other methods commonly known in the art. One exemplary methodology for identifying antibodies having a high specificity for a particular validated protein is the phage display technology.

Phage display libraries for the production of high-affinity antibodies are described in, for example, Hoogenboom, H. R. et al., Immunotechnology (1998) 4(1):1-20; Hoogenboom, H. R., Trends Biotechnol. (1997) 15:62-70 and McGuinness, B. et al., Nature Bio. Technol. (1996) 14:1149-1154 each of which is incorporated herein by reference. Among the advantages of the phage display technology is the ability to isolate antibodies of human origin that cannot otherwise be easily isolated by conventional hybridoma technology. Furthermore, phage display antibodies may be isolated in vitro without relying on an animal's immune system.

Antibody phage display libraries may be accomplished, for example, by the method of McCafferty et al., Nature (1990) 348:552-554 which is incorporated herein by reference. In short, the coding sequence of the antibody variable region is fused to the amino terminus of a phage minor coat protein (pIII). Expression of the antibody variable region-pIII fusion construct results in the antibody's “display” on the phage surface with the corresponding genetic material encompassed within the phage particle.

A validated protein, or fragment thereof suitable for screening a phage library may be obtained by, for example, expression in baculovirus Sf9 cells as described, supra. Alternatively, the validated protein coding region may be PCR amplified using primers specific to the desired region of the validated protein. As discussed above, the validated protein may be expressed in E. coli or yeast as a fusion with one of the commercially available affinity tags.

The resulting fusion protein may then be adsorbed to a solid matrix, e.g., a tissue culture plate or bead. Phage expressing antibodies having the desired anti-validated protein binding properties may subsequently be isolated by successive panning, in the case of a solid matrix, or by affinity adsorption to a validated protein antigen column. Phage having the desired validated protein inhibitory activities may be reintroduced into bacteria by infection and propagated by standard methods known to those skilled in the art. See Hoogenboom, H. R., Trends Biotechnol., supra for a review of methods for screening for positive antibody-pIII phage.

Small Molecules and High-Throughput Screening (HTS) Assays

The present invention also provides small molecule modulators of virus (e.g., HIV, WNV, JEV, SLE, YFV, DEN and HCV) infection, replication, maturation or progression (or of virus-related effects) that may be readily identified through routine application of high-throughput screening (HTS) methodologies. Reviewed by Persidis, A., Nature Biotechnology (1998) 16:488-489. HTS methods generally permit the rapid screening of test compounds, such as small molecules, for therapeutic potential. HTS methodology employs robotic handling of test materials, detection of positive signals and interpretation of data. Such methodologies include, e.g., robotic screening technology using soluble molecules as well as cell-based systems such as the two-hybrid system described in detail above.

A variety of cell line-based HTS methods are available that benefit from their ease of manipulation and clinical relevance of interactions that occur within a cellular context as opposed to in solution. Test compounds are be identified via incorporation of radioactivity or through optical assays that rely on absorbance, fluorescence or luminescence as read-outs. See, e.g., Gonzalez, J. E. et al., Curr. Opin. Biotechnol. (1998) 9(6):624-631 incorporated herein by reference.

HTS methodology is employed, e.g., to screen for test compounds that modulate or block one of the biological activities of a validated protein (i.e., a protein encoded by validated HIV-induced cellular gene expression). For example, a validated protein may be immunoprecipitated from cells expressing the protein and applied to wells on an assay plate suitable for robotic screening. Individual test compounds are contacted with the immunoprecipitated protein and the effect of each test compound on an activity of the validated protein is assessed. For example, if the particular validated protein has kinase activity, the effect of a particular test compound on the kinase is assessed by, e.g., incubating the corresponding immunopreciped protein in contact with the particular test compound in the presence of γ-³²P-ATP in a suitable buffer system, and measuring the incorporation of ³²P.

Both small molecule agonists and antagonists of particular validated proteins (SEQ ID NOS:2, 4, 7, and 9) are encompassed within the scope of the present invention.

In particular embodiments, HIV-infected THP1 or MT-2 cells are used in inventive screening assays for therapeutic compounds (e.g., see EXAMPLE 4, herein below).

Particular embodiments provide a method for inhibiting virus (e.g., HIV, WNV, JEV, SLE, YFV, DEN and HCV) infection and/or replication comprising administration of an src family kinase inhibitor selected from the group consisting of src family kinase-specific antisense RNA, src family kinase-specific siRNA, and a small molecule inhibitor of a src family kinase.

Preferably, the src family kinase is c-yes kinase (SEQ ID NOS:8 and 9). Preferably, the small molecule src family kinase inhibitor is a compound having the structure of Formula I, Formula I(b), or Formula II, or salts thereof:

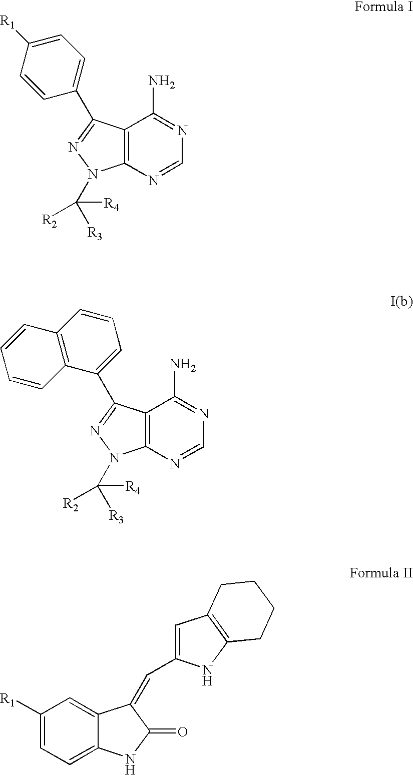

Preferably, for Formula I or I(b), R₁ is halogen or methyl, and R₂, R₃ and R₄ are independently a C₁-C₃ straight or branched alkyl. Preferably, for Formula I, R₁ is —SO₂N(CH₃)₂, or —SO₂NH₂. Preferably, the inhibitor is 4-Amino-5-(4-chlorophenyl)-7-(t-butyl)pyrazolo[3,4-d]pyrimidine (“PP2”).

Preferably, the inhibitor of Formula I(b) is 4-Amino-1-tert-butyl-3-(1′-naphthyl)pyrazolo[3,4-d]pyrimidine (Calbiochem/Merck Biosciences; 529579 PP1 analog) (Bishop, A. C., et al., Nature 407:395, 2000; and Bishop, A. C., et al., J. Am. Chem. Soc. 121:627, 1999).

Preferably, for Formula II, the inhibitor is 2-oxo-3-(4,5,6,7-tetrahydro-1H-indol-2-ylmethylene)-2,3-dihydro-1H-indole-5-sulfonic acid dimethylamide (“SU6656”).

According to particular aspects of the present invention, additional preferred small molecule SFK inhibitors are:

Tetrahydroindole-based indolinones. In particular embodiments, the SFK inhibitor is a tetrahydroindole-based indolinone, or a pharmaceutically acceptable salt thereof (SUGEN, San Francisco, Calif.) (see Tables 1 and 2 of Guan et al., Bioorg. Med. Chem. Lett. 14:187-190, 2004; incorporated by reference herein in its entirety).

Preferably, the tetrahydroindole-based indolinone is a substituted 3-[3-(3-dimethylamino-propyl)-4,5,6,7-tetrahydro-1H-indol-2-ylmethylene]-1,3-dihydro-indole-2-one. Preferably, the tetrahydroindole-based indolinone is 3-[3-(3-dimethylamino-propyl)-4,5,6,7-tetrahydro-1H-indol-2-ylmethylene]-2-oxo-2,3-dyhydro-1H-indole-5-sulphonic acid methylamide (compound 1a of Guan et al., 2004, supra).

Preferably, in these compounds, there is a sulfone group directly attached to the C-5 position of the oxindole ring (i.e., to the oxindole core). Preferably, the C-3′ position of the tetrahydroindole core is substituted (Formula III): 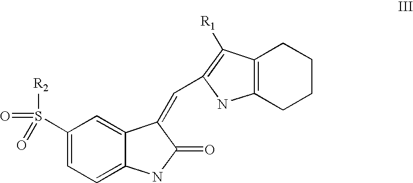
wherein R₂ is C₂H₅ or NHR₃, wherein R₃ is a C1 to C3 linear or branched alkyl moiety, and wherein R₁ is independently —(CH₂)₃N(CH₃)₂, —CH₂N(CH₂CH₂)₂O, —(CH₂)₂N(CH₂CH₂)₂O, —(CH₂)₃N(CH₂CH₂)₂O, or —(CH₂)₃N(CH₂CH₂)₂N CH₃.

Preferably, the substituents at the C-5 position of the oxindole ring are 5-ethylsulfone, or 5-methylsulfonaamide.

Preferably, the C-3′ position substituent is a basic (3-amino)propyl substituent. Preferably, the C-3′ position basic (3-amino)propyl substituent is: —(CH₂)₃N(CH₃)₂; (CH₂)₃N(CH₂CH₂)₂O; or —(CH₂)₃N(CH₂CH₂)₂NCH₃.

Preferably the tetrahydroindole-based indolinone compound is compound: 1a [R₂ is NH CH₃ and R₁ is —(CH₂)₃N(CH₃)₂]; 1i [R₂ is C₂H₅, and R₁ is —(CH₂)₃N(CH₃)₂]; 3f [R₂ is NH CH₃ and R₁ is —(CH₂)₃N(CH₂CH₂)₂O]; 3g [R₂ is NH CH₃ and R₁ is —(CH₂)₃N(CH₂CH₂)₂N CH₃]; or 4g [R₂ is C₂H₅, and R₁ is —(CH₂)₃N(CH₂CH₂)₂N CH₃] of Guan et al., 2004, supra.

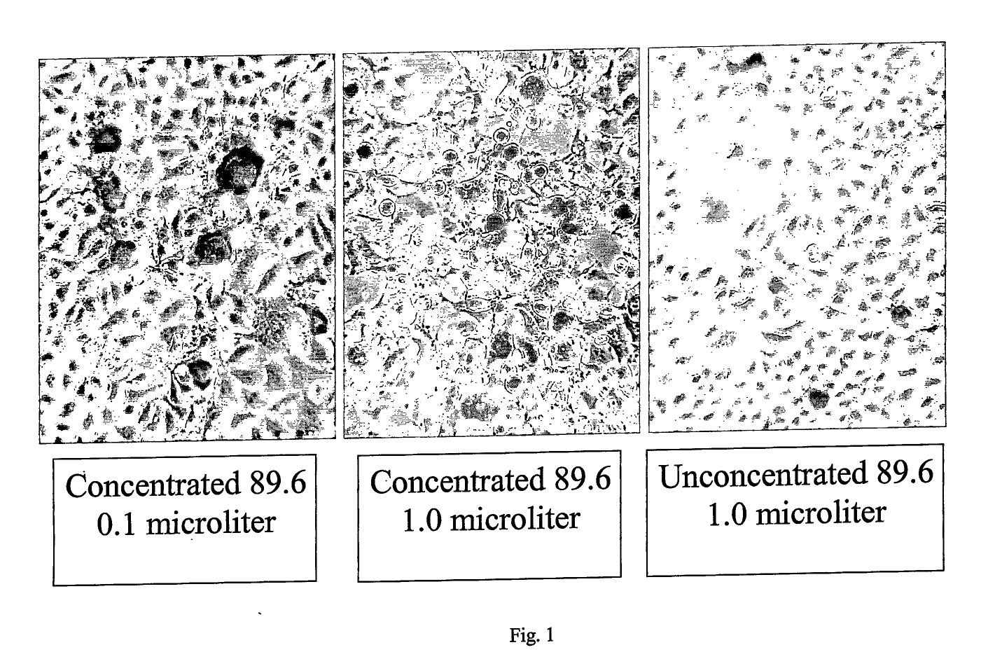

Pyrido-[2,3-d]pyrimidine-based compounds. In alternate embodiments, the SFK inhibitor is compound with a pyrido-[2,3-d]pyrimidine core structure, or a pharmaceutically acceptable salt thereof. Preferably, the pyrido-[2,3-d]pyrimidine compound is PD173955 (Formula IV) (Parke-Davis/Warner-Lambert, Ann Arbor, Mich.) (see FIG. 1 of Moasser et al., Cancer Research 59:6145-6152, 1999; incorporated by reference herein in its entirety):

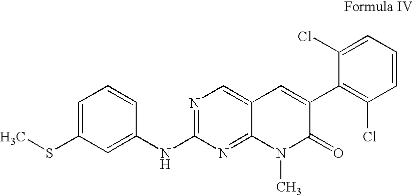

5,7-diphenyl-pyrolo[2,3-d]pyrimidine-based compounds. In additional alternate embodiments, the SFK inhibitor is a substituted 5,7-diphenyl-pyrolo[2,3-d]pyrimidine, or a pharmaceutically acceptable salt thereof (Novartis, Basel, Switzerland) (see FIG. 1 of Missbach et al., Bone 24:437-449, 1999; incorporated by reference herein in its entirety).

Preferably, the substituted 5,7-diphenyl-pyrolo[2,3-d]pyrimidine is one of the compounds 1-8 of Missbach et al (Id) according to Formula V: 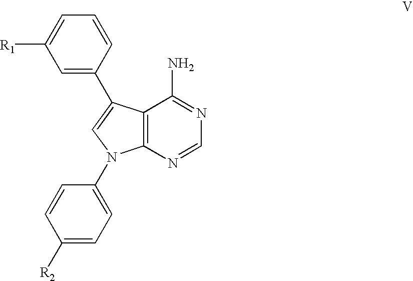
Wherein R₁ is either H or —OCH₃, and wherein R₂ is independently —(CH₂)₂OH, —CH₂COOH, —(CH₂)₂N(CH₃)₂, —(CH₂)₂NH(CH₂)₂OH, —(CH₂)₂NCH₃(CH₂)₂OCH₃, —(CH₂)₂N(CH₂CH₂)₂NCH₃, or —(CH₂)₂N(CH₂CH₂)₂CHOH.

Preferably the substituted 5,7-diphenyl-pyrolo[2,3-d]pyrimidine is CGP77675 (i.e., where R₁ is —OCH₃R₂, and R₂ is —(CH₂)₂N(CH₂CH₂)₂CHOH; compound 8 of Missbach et al) (Id).

Methods for Assessing the Efficacy of Modulators of either Virus (e.g. HIV, WNV, JEV, SLE, YFV, Den and HCV)-Induced Gene Expression or of Biological Activity Encoded Thereby

Inventive modulators or compounds, whether antisense molecules, siRNA, or ribozymes, proteins and/or peptides, antibodies and/or antibody fragments or small molecules, that are identified either by one of the methods described herein or via techniques that are otherwise available in the art, may be further characterized in a variety of in vitro, ex vivo and in vivo animal model assay systems for their ability to modulate or inhibit HIV-induced gene expression or biological activity. As discussed in further detail in the EXAMPLES provided below, particular inventive modulators of virus-induced gene expression are antisense, siRNA or and small molecule inhibitors effective in reducing virus-induced cellular gene expression levels. Thus, the present invention describes, teaches and supports methods that permit the skilled artisan to assess the effect of candidate modulators and inhibitors.

For example, candidate modulators or inhibitors of virus-induced gene expression are tested by administration of such candidate modulators to cells that express the respective virus-induced genes and gene products (e.g., HIV-infected THP1 or MT-2 cells in the inventive HIV replication assay system). Virus-infected mammalian cells may also be engineered to express a given virus-induced gene or recombinant reporter molecule introduced into such cells with a recombinant virus-inducible gene plasmid construct.

Effective modulators of virus (e.g., HIV, WNV, JEV, SLE, YFV, DEN and HCV)-induced gene expression that are inhibitors will be effective in reducing the levels of virus-induced gene mRNA as determined, e.g., by Northern blot or RT-PCR analysis. For a general description of these procedures, see, e.g., Sambrook et al., Molecular Cloning: A Laboratory Manual Cold Spring Harbor Press (1989) and Molecular Biotechnology: Principles and Applications of Recombinant DNA, ASM Press (ed. Glick, B. R. and Pasternak, J. J. 1998) incorporated herein by reference. The effectiveness of a given candidate antisense molecule may be assessed by comparison with a control ‘antisense’ molecule (e.g., a reverse complement control oligonucleotide, corresponding in orientation and size to the coding sequence complementary to the candidate antisense molecule) known to have no substantial effect on virus-induced gene expression when administered to a mammalian cell. Exemplary control molecules include virus-inducible gene sequence-specific reverse complement oligonucleotides corresponding to one of the inventive antisense molecules described herein above, or to preferred representative thereof (e.g., reverse complement control oligonucleotides for SEQ ID NOS:10-14).

In alternate embodiments of the present invention, the effect of modulators and inhibitors of virus-induced gene expression on the rate of DNA synthesis after challenge with a radiation or chemotherapeutic agent may be assessed by, e.g., the method of Young and Painter. Hum. Genet. (1989) 82:113-117. Briefly, culture cells may be incubated in the presence of ¹⁴C-thymidine prior to exposure to, e.g., X-rays. Immediately after irradiation, cells are incubated for a short period prior to addition of ³H-thymidine. Cells are washed, treated with perchloric acid and filtered (Whatman GF/C). The filters are rinsed with perchloric acid, 70% alcohol and then 100% ethanol; radioactivity is measured and the resulting ³H/¹⁴C ratios used to determine the rates of DNA synthesis.

Modulators or inhibitors of virus (e.g., HIV, WNV, JEV, SLE, YFV, DEN and HCV)-induced gene expression effective in modulating or reducing virus-induced cellular gene expression by one or more of the methods discussed above are further characterized in vivo for efficacy one or more available art-recognized animal model systems (e.g., SIV model). Various animal model systems for study of cancer and genetic instability associated genes are disclosed in, for example, Donehower, L. A. Cancer Surveys (1997) 29:329-352 incorporated herein by reference. In particular, various art-recognized animal model systems for testing PMO antisense oligonucleotide agents, including xenograft murine models are discussed Devi, Current Opinion in Molecular Therapeutics, 4:138-148, 2002, incorporated by reference herein.

Pharmaceutical Compositions

The antisense oligonucleotides and ribozymes of the present invention are synthesized by any method known in the art for ribonucleic or deoxyribonucleic nucleotides. For example, the oligonucleotides are prepared using solid-phase synthesis such as in an Applied Biosystems 380B DNA synthesizer. Final purity of the oligonucleotides is determined as is known in the art.

The antisense oligonucleotides identified using the methods of the invention modulate cancer cell proliferation, including anchorage-independent proliferation, and also modulate virus (e.g., HIV, WNV, JEV, SLE, YFV, DEN and HCV)-mediated phenotypic changes.

Therefore, pharmaceutical compositions and methods are provided for interfering with virus infection, replication, maturation or progression, or for virus-related conditions or diseases, comprising contacting tissues or cells with one or more of antisense oligonucleotides or siRNA identified using the methods of the invention. Preferably, an antisense oligonucleotide having one of SEQ ID NOS:10-14, or preferably SEQ ID NOS:10-13, is administered. Preferably, the antisense oligonucleotide is a PMO antisense oligomer (PMO).

The methods and compositions may also be used to treat other HIV-associated conditions and disorders known in the art.

The invention provides pharmaceutical compositions of antisense oligonucleotides, siRNA and ribozymes complementary to validated virus-induced cellular gene mRNA gene sequences, corresponding to SEQ ID NOS:1, 3, 5, 6, and 8, and complements thereof as active ingredients for therapeutic application. These compositions can also be used in the methods of the present invention. Where required the compounds are nuclease resistant. In general the pharmaceutical composition for modulating virus-mediated cellular proliferation or phenotype in a mammal includes an effective amount of at least one antisense oligonucleotide (or siRNA agent, etc) or small molecule as described above needed for the practice of the invention, or a fragment thereof shown to have the same effect, and a pharmaceutically physiologically acceptable carrier or diluent.

Particular embodiments provide a method for reducing virus (e.g., HIV, WNV, JEV, SLE, YFV, DEN and HCV) infection, replication, maturation, or progression in a subject comprising administering an amount of an antisense oligonucleotide (or siRNA agent) of the invention effective to reduce said virus infection and/or replication Preferably the antisense oligomer (siRNA) is based on one of SEQ ID NOS:1, 3, 5, 6, and 8. More preferably the antisense oligonucleotide is one of SEQ ID NOS:10-13.

The pharmaceutical composition for inhibiting virus (e.g., HIV, WNV, JEV, SLE, YFV, DEN and HCV) infection, replication, maturation or progression in cells in a mammal consists of an effective amount of at least one active ingredient selected from siRNA agents, or antisense oligonucleotides complementary to the virus-induced cellular gene mRNA, including the entire virus-induced gene mRNA or having shorter sequences as set forth in SEQ ID NOS:15-21, and a pharmaceutically acceptable carrier or diluent. Combinations of the active ingredients are contemplated and encompassed within the scope of the invention.

The compositions can be administered orally, subcutaneously or parenterally including intravenous, intraarterial, intramuscular, intraperitoneally, and intranasal administration as well as intrathecal and infusion techniques as required by the cells being treated. For delivery within the CNS, intrathecal delivery can be used with for example an Ommaya reservoir or other methods known in the art. The pharmaceutically acceptable carriers, diluents, adjuvants and vehicles as well as implant carriers generally refer to inert, non-toxic solid or liquid fillers, diluents or encapsulating material not reacting with the active ingredients of the invention. Cationic lipids may also be included in the composition to facilitate oligonucleotide uptake. Implants of the compounds are also useful. In general, the pharmaceutical compositions are sterile.

In particular method aspects of the present invention, virus-related cells are contacted with an efficacious amount of the bioactive antisense oligonucleotide (or siRNA agent) for the virus-induced cellular gene mRNA or a fragment thereof shown to have substantially the same effect. In an embodiment, the mammal to be treated is human but other mammalian species can be treated in veterinary applications.

Bioactivity, relating to a particular oligonucleotide modulator, refers to biological activity in the cell when the oligonucleotide modulator is delivered directly to the cell and/or is expressed by an appropriate promotor and active when delivered to the cell in a vector as described below. Nuclease resistance of particular modulators is provided by any method known in the art that does not substantially interfere with biological activity as described herein.

Significantly, PMO chemistry is not RNase H competent (discussed in Devi, Current Opinion in Molecular Therapeutics, 4:138-148, 2002).

“Contacting the cell” refers to methods of exposing, delivery to, or ‘loading’ of a cell of antisense oligonucleotides whether directly or by viral or non-viral vectors, and where the antisense oligonucleotide is bioactive upon delivery. The method of delivery will be chosen for the particular cell type being treated. Parameters that affect delivery can include the cell type affected and its location as is known in the medical art.

The treatment generally has a length proportional to the length of the disease process and drug effectiveness and the patient species being treated. It is noted that humans are treated generally longer than the Examples exemplified herein, which treatment has a length proportional to the length of the disease process and drug effectiveness. The doses may be single doses or multiple doses as determined by the medical practitioners and treatment courses will be repeated as necessary until diminution of the disease is achieved. Optimal dosing schedules may be calculated using measurements of drug accumulation in the body. Practitioners of ordinary skill in the art can readily determine optimum dosages, dosing methodologies, and repetition rates. Optimum dosages may vary depending on the relative potency of the antisense oligonucleotide (or siRNA agent), and can generally be determined based on values in in vitro and in vivo animal studies and clinical trials. Variations in the embodiments used may also be utilized. The amount must be effective to achieve improvement including but not limited to decreased infection, viral replication, or to improved survival rate or length or decreased drug resistance or other indicators as are selected as appropriate measures by those skilled in the art.

Although particular inventive antisense oligonucleotides (or siRNA agents) may not completely abolish viral infection and/or replication, or other virus-induced effects in vitro, as exemplified herein, these antisense compounds and agents are nonetheless clinically useful where they inhibit virus-related infection, replication, maturation, progression, etc., enough to allow complementary treatments, such as chemotherapy, radiation therapy, or other drug therapies to be effective or more effective. The pharmaceutical compositions of the present invention therefore are administered singly or in combination with other drugs, such as, in the case of AIDS for example, HIV inhibitory agents (AZT, etc.), cytotoxic agents, immunotoxins, alkylating agents, anti-metabolites, antitumor antibiotics and other anti-cancer drugs and treatment modalities that are known in the art.

Cocktails of antisense inhibitors directed against several virus-induced gene sequences are contemplated and within the scope of the present invention.

The composition is administered and dosed in accordance with good medical practice taking into account the clinical condition of the individual patient, the site and method of administration, scheduling of administration, and other factors known to medical practitioners. The “effective amount” for virus inhibition is thus determined by such considerations as are known in the art. The pharmaceutical composition may contain more than one embodiment or modulator of the present invention.

The nucleotide sequences of the present invention can be delivered either directly or with viral or non-viral vectors. When delivered directly the sequences are generally rendered nuclease resistant. Alternatively, the sequences can be incorporated into expression cassettes or constructs such that the sequence is expressed in the cell. Generally, the construct contains the proper regulatory sequence or promoter to allow the sequence to be expressed in the targeted cell.

Once the oligonucleotide sequences are ready for delivery, they can be introduced into cells as is known in the art (see, e.g., Devi, Current Opinion in Molecular Therapeutics, 4:138-148, 2002). Transfection, electroporation, fusion, liposomes, colloidal polymeric particles and viral vectors as well as other means known in the art may be used to deliver the oligonucleotide sequences to the cell. The method selected will depend at least on the cells to be treated and the location of the cells and will be known to those skilled in the art. Localization can be achieved by liposomes, having specific markers on the surface for directing the liposome, by having injection directly into the tissue containing the target cells, by having depot associated in spatial proximity with the target cells, specific receptor mediated uptake, viral vectors, or the like.

Administration and clinical dosing of PMO antisense therapeutic agents is discussed, for example, in Devi, supra, and in Arora et al. Journal of Pharmaceutical Sciences, 91:1009-1018, 2001, both incorporated by reference herein.

The present invention provides, in particular aspects, vectors comprising an expression control sequence operatively linked to the oligonucleotide sequences of the invention. The present invention further provides host cells, selected from suitable eukaryotic and prokaryotic cells, which are transformed with these vectors as necessary. Such transformed cells allow the study of the function and the regulation of malignancy and the treatment therapy of the present invention.

Vectors are known or can be constructed by those skilled in the art and should contain all expression elements necessary to achieve the desired transcription of the sequences. Other beneficial characteristics can also be contained within the vectors such as mechanisms for recovery of the oligonucleotides in a different form. Phagemids are a specific example of such beneficial vectors because they can be used either as plasmids or as bacteriophage vectors. Examples of other vectors include viruses such as bacteriophages, baculoviruses and retroviruses, DNA viruses, liposomes and other recombination vectors. The vectors can also contain elements for use in either prokaryotic or eukaryotic host systems. One of ordinary skill in the art will know which host systems are compatible with a particular vector.

The vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al., BioTechniques (1986) 4:504-512 and include, for example, stable or transient transfection, lipofection, electroporation and infection with recombinant viral vectors.

Recombinant methods known in the art can also be used to achieve the antisense inhibition (or siRNA mediated inhibition) of a validated target nucleic acid. For example, vectors containing antisense nucleic acids can be employed to express an antisense message to reduce the expression of the validated target nucleic acid and therefore its activity.

The present invention also provides a method of evaluating if a compound inhibits transcription or translation of an virus-induced cellular gene sequence, and thereby modulates (i.e., reduces) viral infection, replication, cell proliferation or phenotypic differentiation, comprising transfecting a cell with an expression vector comprising a nucleic acid sequence encoding a HIV-induced cellular gene sequence, the necessary elements for the transcription or translation of the nucleic acid; administering a test compound; and comparing the level of expression of the HIV-induced cellular gene sequence with the level obtained with a control in the absence of the test compound. Alternatively, as is shown in the EXAMPLES herein, such an expression vector is not required, and test compounds are administered to virus-infected cells, such as, for example, HIV-infected THP1 or MT-2 cells.

The present invention provides detectably labeled oligonucleotides for imaging virus (e.g., HIV, WNV, JEV, SLE, YFV, DEN and HCV)-induced cellular gene sequences (polynucleotides) within a cell. Such oligonucleotides are useful for determining if gene amplification has occurred, for assaying the expression levels in a cell or tissue using, for example, in situ hybridization as is known in the art, and for diagnostic and/or prognostic purposes.

Diagnostic and/or Prognostic Assays for Virus (e.g., HIV, WNV, JEV, SLE, YFV, DEN and HCV) and Virus-Related Conditions or Diseases

The present invention provides for diagnostic and/or prognostic cancer assays based on differential measurement of validated virus (e.g., HIV, WNV, JEV, SLE, YFV, DEN and HCV)-induced gene expression. Preferred validated virus-induced gene sequences are represented herein by SEQ ID NOS:1, 3, 5, 6, and 8, and complements thereof, along with the corresponding gene products SEQ ID NOS:2, 4, 7 and 9, and combinations thereof.

Typically, such assays involve obtaining a tissue sample from a test tissue, performing an assay to measure expression of at least one validated virus-induced gene product (e.g., mRNA or protein encoded thereby) derived from the tissue sample, relative to a control sample, and making a diagnosis or prognosis based, at least in part, thereon.

In particular embodiments the present inventive oligomers, such as those based on validated SEQ ID NOS:1, 3, 5, 6, and 8, and complements thereof, or preferably SEQ ID NOS:10-13, or arrays comprising any of the preceding validated sequences or gene products, as well as a kit based thereon are useful for the diagnosis and/or prognosis of virus infection and/or replication, or other virus-related cell disorders, conditions or diseases.

The present invention moreover relates to a method for manufacturing a diagnostic agent and/or therapeutic agent for virus (e.g., HIV, WNV, JEV, SLE, YFV, DEN and HCV) diagnosis and/or therapy of virus-related conditions or diseases, the diagnostic agent and/or therapeutic agent being characterized in that at least one inventive validated modulator of virus-induced gene expression is used for manufacturing it, possibly together with suitable additives and ancillary agents.

Diagnostic kits are also contemplated, comprising at least one primer and/or probe specific for a validated virus (e.g., HIV, WNV, JEV, SLE, YFV, DEN and HCV)-induced cellular gene sequence according to the present invention, possibly together with suitable additives and ancillary agents.

While the present invention has been described with specificity in accordance with certain of its preferred embodiments, the following EXAMPLES serve only to illustrate the invention and are not intended to limit the invention.

### EXAMPLE 1

### HIV-Infected THP1 and MT-2 Cells are a Valid In Vivo Model System for HIV Replication

The HIV-1 strain used in the model system was the 89.6 strain. This is a dual tropic (X4/R5) HIV strain, meaning that it can infect cells utilizing CD4 and either the CXCR4 or the CCR5 co-receptor. Thus, both T cells (e.g., MT-2) and macrophages (e.g., THP-1) are susceptible to infection by the same virus strain. HIV-1 89.6 was originally provided by the investigator who isolated and characterized it, Dr Ronald Collman (Collman et al, J. Virology 66:7517, 1992). Applicant's expanded the virus by culture in PBMC, and concentrated it for use in the inventive system as described in EXAMPLE 2, herein below.

HIV-infected THP-1 and MT-2 cells. The cell lines selected for use herein include the monocyte line THP-1 and the T cell leukemia cell line MT-2.

The THP-1 cell line is CD4+, highly permissive for HIV infection, and has been used by numerous investigators for studying various aspects of HIV biology, either prior to or in concert with examination of primary cells. Reference to the HIV literature over the past 6 months reveals several studies of HIV biology and therapeutics that underscore this point: Briquet & Vaquero, Virology 292:177-84, 2002 (Immunolocalization studies of an antisense protein in HIV-1-infected cells and viral particles); Branch, D. R. et al. Aids 16:309-19, 2002 (VPAC1 is a cellular neuroendocrine receptor expressed on T cells that actively facilitates productive HIV-1 infection); Nguyen & Taub, J Immunol 168:4121-6 (CXCR4 function requires membrane cholesterol: implications for HIV infection); Ho, W. Z., et al., Faseb J 16:616-8, 2002 (HIV enhances substance P expression in human immune cells); Hayes, M. M., et al., J. Biol Chem 277:16913-9, 2002 (Peroxisome proliferator-activated receptor gamma agonists inhibit HIV-1 replication in macrophages by transcriptional and post-transcriptional effects); Lenardo, M. J. et al. J Virol 76:5082-93, 2002 (Cytopathic killing of peripheral blood CD4(+) T lymphocytes by human immunodeficiency virus type 1 appears necrotic rather than apoptotic and does not require env.); Bolton, D. L. et al. J Virol 76:5094-107, 2002 (Death of CD4(+) T-cell lines caused by human immunodeficiency virus type 1 does not depend on caspases or apoptosis); Alfano, M., et al., Proc Natl Acad Sci USA 99:8862-7, 2002 (Urokinase-urokinase receptor interaction mediates an inhibitory signal for HIV-1 replication); Wu, L., Martin, et al., J Virol 76:5905-14, 2002 (Functional evaluation of DC-SIGN monoclonal antibodies reveals DC-SIGN interactions with ICAM-3 do not promote human immunodeficiency virus type 1 transmission); Mautino & Morgan, Hum Gene Ther 13:1027-37, 2002 (Enhanced inhibition of human immunodeficiency virus type 1 replication by novel lentiviral vectors expressing human immunodeficiency virus type 1 envelope antisense RNA).

Reduced biological noise and the capacity to infect the majority of cells in culture are both important details for a relevant array-based analysis of the effect of HIV infection, and both of these parameters have been achieved by the use of this CD4+ cell line. THP-1 cells were obtained from the American Type Culture Collection (Manassas, Va.). They are currently available for our use as multiple age- and passage-matched cryopreserved aliquots. All cell lines are maintained as suspension cultures in vented T75 tissue culture flasks at densities of 5×10⁶ to 5×10⁷ cells/ml in RPMI supplemented with 2 mM glutamine, penicillin/streptomycin and 10% FBS. Following treatment with TPA, THP-1 cells differentiate into adherent macrophage-like cells and are cultured in 60 mm tissue culture-treated dishes.

Additionally, the T cell line “MT-2” was used. MT-2 is a human T cell leukemia cell line that can be grown in suspension and, like THP-1 cells, are very susceptible to acute infection with HIV. The cells can be efficiently loaded with antisense oligonucleotides. In addition, they have been shown by other investigators to provide a sensitive and reproducible system to test antiviral agents (see, e.g., Haertle et al, J. Biol. Chem. 263:5870-5875, 1988). MT-2 cells are available through the NIH AIDS Research and Reference Reagent Program.

HIV-infected THP-1 cells. HIV-infected THP-1 cells were used as an in vitro model for examining cellular gene expression during the HIV replication cycle. The HIV-1 89.6 was initially grown in PBMC, and concentrated using Amicon membranes prior to use for infection of THP-1 cells. Briefly, as the goal of the present studies was to profile cellular gene expression over the course of a complete viral replication cycle, the protocol called for synchronous infection of the majority of cells in culture, with multiple samplings over the first 48 hours post-infection (PI). Since the number of target cells required to yield sufficient RNA for gene profiling at multiple times PI is high (>10⁸), synchronous infection of such cell numbers requires high titer virus stocks. However, virus titers derived from PBMC supernatants are typically only in the region of 1×10⁵ to 1×10⁶ pfu/ml. Additionally, such supernatants may also contain cellular growth factors with the capacity to influence gene expression independent of virus-induced effects.

To circumvent these obstacles, PBMC-derived stocks of HIV-1 89.6 were concentrated 20-fold using Amicon Ultra-15 centrifugal filter devices (Millipore, Bedford Mass.). This procedure yielded high titer virus (±1×10⁷ pfu/ml) that could be resuspended in medium free from host-cell derived factors.

The potency of concentrated virus stocks is exemplified in FIG. 1 which illustrates MAGI cells (HeLa-CD4⁺-HIV LTR-β-gal) infected with 1 μl or 0.1 μl of concentrated virus as compared to 1 μl of unconcentrated virus. MAGI cells infected with 0.1 μl of unconcentrated 89.6 exhibited no sign of infection. MAGI cells are Hela CD4 cells stably transfected with the β-galactosidase gene under the control of the HIV LTR. When MAGI cells are productively infected with HIV, β-galactosidase expression is induced by tat-transactivation and the number of blue cells revealed by staining is a measure of virus titer.

The left and center panels of FIG. 1 show the number of blue-staining cells increasing in a dose responsive manner with the use of concentrated HIV-1 89.6 stocks.

### EXAMPLE 2

### Nucleic Acid Microarray Technology was Used for Gene Expression Profiling of HIV-Infected THP-1 Cells to Identify Cellular Genes Whose Expression is Regulated by HIV

Nucleic Acid Microarray Data Analysis. Cellular genes involved in HIV-1 replication were identified by using DNA microarrays to examine the differential gene expression profiles of THP-1 cells before and after HIV-infection.

For RNA isolation and fluorescent labeling, two RNA probe samples from THP-1 cells, independently infected with KSHV, and two independent uninfected RNA probe samples were prepared. Briefly, THP1 monocytes infected with HIV isolate MN or with 89.6 were harvested at 2, 4, 6, 8, 10, and 12 hours post infection (PI). Uninfected cells were harvested in parallel.

Generally, RNA was isolated using the RNeasy™ RNA isolation kit (QIAGEN Inc., Valencia, Calif.). After DNase treatment and another round of RNeasy purification, labeled cDNA was prepared as described previously (see Salunga et al., In M. Schena (ed.), DNA microarrays. A practical approach; Oxford Press, Oxford, United Kingdom, 1999; and see Simmen et al., Proc. Natl. Acad. Sci. USA 98:7140-7145, 2001). Briefly, double-stranded cDNA was selectively synthesized from the RNA samples. Biotin-labeled cRNA was produced from the cDNA by in vitro transcription (IVT) using methods well known in the art.

For expression profile screening, the biotin-labeled cRNA probe preparations were fragmented and hybridized to Affymetrix (Santa Clara, Calif.) U133A and U133B arrays or to U95A arrays (Affymetrix U133A, U133B and U95A GeneChip® arrays). The Human Genome U133 (HG-U133) set, consists of two GeneChip® arrays, and contains almost 45,000 probe sets representing more than 39,000 transcripts derived from approximately 33,000 well-substantiated human genes (Affymetrix technical information). The set design uses sequences selected from GenBank®, dbEST, and RefSeq (Id).

The Affymetrix GeneChip® platform was chosen for these studies as it is the industry leader in terms of array content, platform stability and data quality. Images of the arrays were analyzed using the Affymetrix microarray analysis suite software, MAS. This software package is used for converting images to raw numerical data, and direct comparisons between control and experimental samples. When making such comparisons, MAS provides robust statistical algorithms for determining changes in expression between the two samples, along with p-values and confidence limits on such changes. For each probe set, MAS records whether there was no change, increased expression or decreased expression. To determine if the number of gene expression changes in common between two or more experiments is significant, we routinely compared the number of genes in such lists to the number expected if the experiments were independent.

Each of the THP-1 infected/uninfected sample comparisons resulted in multiple probe sets with increased expression, with some sets showing increased expression in both infections. Increased or decreased expression was based on ‘calls’ from MAS software which typically corresponds to about a two-fold change.

Genes with increased transcription in at least two adjacent time points across the two infections were considered for possible validation studies using PMOs. Genes with evidence of induction due to interferon, in separate experiments conducted in endothelial cells were excluded from consideration. Annotation from the Gene Ontology consortium was compiled for the remaining genes. Those without informative annotation were dropped from further consideration.

Approximately 20 genes were selected from the remaining list for testing as potential anti-viral targets with PMO-AS.

Representative microarray expression data. TABLE 1 (see herein, next page) shows expression data obtained according to the present invention for the HMG20B, HRH1, NP, YES and ARF1 gene sequences using Affymetrix U133 arrays as indicated. Expression is presented as “fold-increase” in signal for two to four independent infected/mock infected comparisons, as described herein above.

TABLE 1
U133 microarray expression data for particular KSHV-induced gene sequences.

| | | | Fold Increase | Fold Increase | Fold Increase | Fold Increase | | |
|---|---|---|---|---|---|---|---|---|
| | | Affymetrix | T89602 × | T89604 × | T89606 × | T89608 × | FC_T89610 × | FC_T89612 × |
| GENE | ARRAY | Probe Set | 067TMK 2 hrs | 067TMK 4 hrs | 067TMK 6 hrs | 067TMK 8 hrs | 067TMK 10 | 067TMK 12 |
| HMG20B | UI33A | 209113_s_at | 11.31 | 3.03 | 4.29 | 6.06 | −1.52 | −1.74 |
| HRH1 | UI33A | 205579_at | 2.64 | 2.64 | 2 | 1.74 | 1.52 | 1.87 |
| HRH1 | UI33A | 205579_at | 2.64 | 2.64 | 2 | 1.74 | 1.52 | 1.87 |
| NP | UI33A | 201695_s_at | 1.87 | 2.14 | 2.64 | 3.03 | 2.3 | 1.52 |
| NP | UI33A | 201695_s_at | 1.87 | 2.14 | 2.64 | 3.03 | 2.3 | 1.52 |
| YES | U133A | 202932_at | 1 | 1.41 | 1.32 | 1.15 | 1.52 | 2 |
| YES | U133A | 202933_s_at | 1.07 | 1.15 | −1.07 | −1.07 | 1.74 | 1.32 |
| ARF1 | U133A | 208750_s_at | 6.06 | 4 | 2.46 | 6.06 | 1.07 | 1.07 |
| ARF1 | U133A | 208750_s_at | 6.06 | 4 | 2.46 | 6.06 | 1.07 | 1.07 |

### EXAMPLE 3

### Target Validation; Genes Necessary for Virally-Induced Morphological Changes in HIV-Infected THP-1 and MT-2 Cells were Identified/Validated Using Antisense PMOs

Antisense Phosphorodiamidate Morpholino Oligomers (PMOs). PMOs (see, e.g., Summerton, et al., Antisense Nucleic Acid Drug Dev. 7:63-70, 1997; and Summerton & Weller, Antisense Nucleic Acid Drug Dev. 7:187-95, 1997) are a class of antisense drugs developed for treating various diseases, including cancer. For example, Arora et al. (J. Pharmaceutical Sciences 91:1009-1018, 2002) demonstrated that oral administration of c-myc-specific and CYP3A2-specific PMOs inhibited c-myc and CYP3A2 gene expression, respectively, in rat liver by an antisense mechanism of action. Likewise, Devi G. R. (Current Opinion in Molecular Therapeutics 4:138-148, 2002) discusses treatment of prostate cancer with various PMO therapeutic agents). See also recent reviews by Milhavet et al., and by Gitlin et al (Milhavet et al Pharmacological Reviews 55:629-648, 2003; and Gitlin et al., J. Virol. 77:7159-7165, 2003; incorporated herein by reference).

PMOs were designed and used, according to the present invention to silence genes identified as being consistently up-regulated in HIV-infected THP-1 and MT-2 cells. PMO-AS (PMO antisense) are about 15-18 base pair oligonucleotides complementary to a specific mRNA start codon that prevent message translation through steric hindrance at the ribosome binding site (Ghosh, et al. Methods in Enzymology 313:135-143, 2000). PMOs do not activate RNAse H, (Id). Typically, it is preferable and sufficient to target the region of the start codon to block translation, but, as discussed herein above, other mRNA regions, both coding and non-coding can be effectively targeted according to the present invention.

Antisense Gene Silencing using PMOs. Genes identified as being consistently up-regulated in HIV-infected THP-1 cells in the above described nucleic acid microarray/gene expression profiling experiments were further analyzed to identify those necessary for viral replication. Silencing of such genes substantially reduced HIV replication relative to controls, as measured by p24 gag ELISA assays (see TABLE 2 below), and validated these cellular gene targets for respective therapeutic methods and compositions for blocking HIV replication, and thus HIV-related conditions and diseases.

Introduction of antisense PMO into HIV-infected THP-1 or MT-2 cells. Antisense PMO molecules, for delivery purposes, are typically converted to a paired duplex together with a partially complementary cDNA oligonucleotide in the weakly basic delivery reagent ethoxylated polyethylenimine (EPEI) (Summerton, supra). The anionic complex binds to the cell surface, is taken up by endocytosis and eventually released into the cytosol. A protocol for optimum uptake of antisense PMO in THP-1 and MT-2 cells was developed using a modification of the EPEI method. PMO-AS were obtained from GeneTools (Genetools, LLC, One Summerton Way, Philomath, Oreg. 97370).

Briefly, loading into target cells was accomplished by complexing the PMO with a proprietary loading reagent, Ethoxylated Polyethylenimine (EPEI). Target cells (MT-2 or THP-1) were introduced into snap cap conical tubes (5×10⁵ cells/tube) in 900 μl serum free RPMI and the EPEI-PMO complex was added for 4 hours at 37° C. in a 7% CO₂ atmosphere. The PMO-EPEI complex was prepared by diluting 5 μl PMO in 40 μl sterile RNase-free water, adding 5 μl EPEI, vortexing and incubating the sample for 20 minutes at room temperature. The sample was then mixed with 50 μl serum-free RPMI and added to the target cells to yield a final volume of 1 ml. At the end of the loading period, cells were gently pelleted and the PMO solution was removed by aspiration. After a further wash, cells were incubated in complete RPMI for 12 hrs prior to HIV infection to allow for recovery and initiation of PMO action.

HIV-1 Infection. PMO-treated THP-1 or MT-2 cells were infected with 50 μl of concentrated HIV-1 89.6 diluted to 1 ml in serum-free RPMI. The following control cultures were identically infected: (i) to control for any non-specific effect of the loading protocol, cells exposed to EPEI alone during the loading procedure; (ii) as a positive control for normal HIV infection, cells that were not exposed to either EPEI or EPEI-PMO; (iii) as a control for HIV inhibition, cells infected with HIV in the presence of 2 μM AZT and maintained in AZT throughout.

Cells were exposed to HIV for 4 hours. The inoculum was removed by 3 cycles of pelleting and rinsing. The cells were resuspended in 1.5 ml of complete RPMI and 0.5 ml immediately removed at Time 0 to leave a final volume of 1 ml. The Time 0 sample served as a measure of residual virus. At each harvest time (T1 through T4), 0.5 ml of supernatant was removed and replaced with an equal volume of complete RPMI. Samples were stored at −80° C. until evaluation using a p24 ELISA assay (described below). Each experimental variable and control was performed in duplicate. T1 through T4 typically corresponded to 16, 24, 40 and 48 hrs PI, respectively.

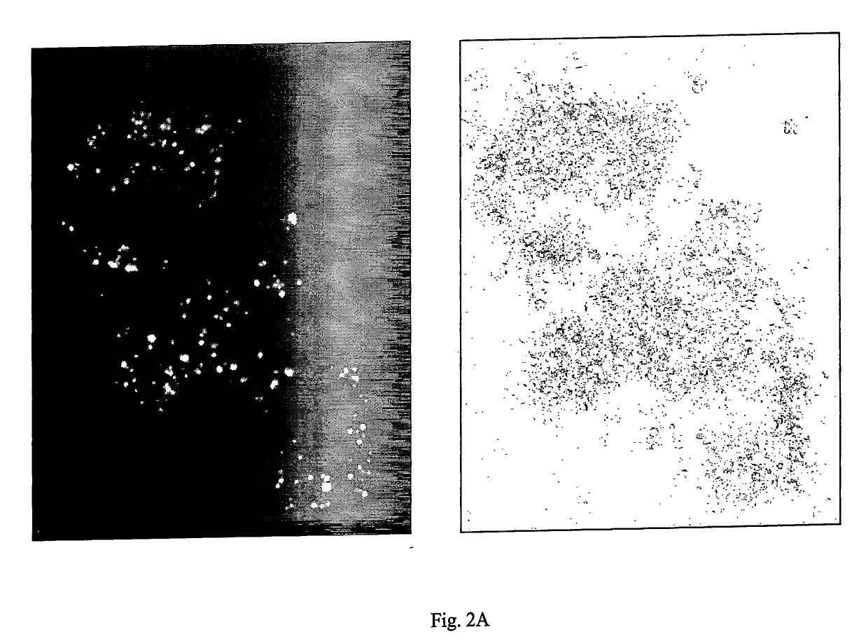

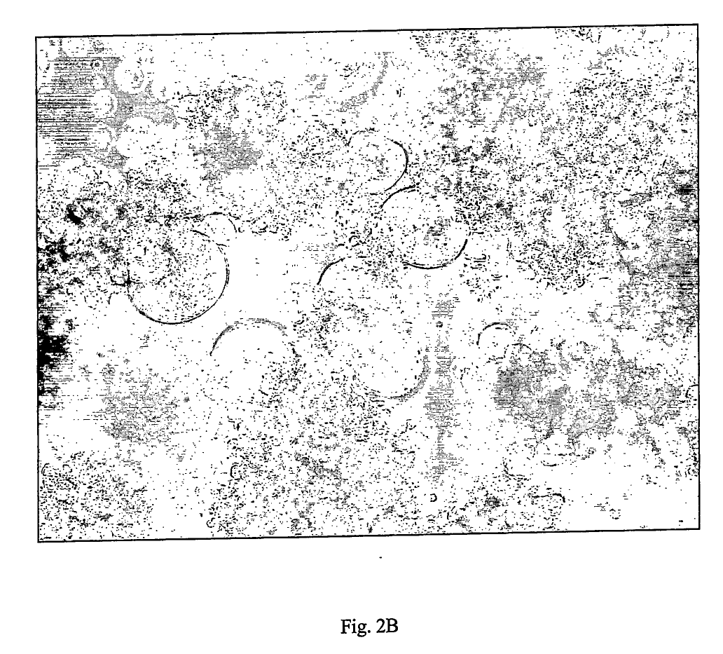

Cellular distribution of introduced FITC-labeled POM antisense molecules. To ensure the success of PMO loading and HIV infection for each assay, cells cultured in 35 mm tissue culture plates were loaded with a FITC tagged PMO, or infected with HIV, and monitored microscopically. FIG. 2a (left panel) illustrates a representative fluorescent image of FITC-labeled PMO antisense uptake by MT-2 cells; that is, successful delivery of a FITC-tagged PMO to MT-2 cells. The right panel show results with the EPEI only control. Therefore, the introduced PMO antisense oligomers were readily taken up by MT-2 cells, distributed within the cytosol, and determined to be stable over the relevant time periods in MT-2 cells.

FIG. 2B illustrates a typical HIV control (no PMO AS) where extensive HIV-induced syncytia are seen in HIV infected MT-2 at 48 hrs PI.

Validation of KSHV-induced gene sequences; HIV-1 p24 antigen ELISA. To monitor the effect of PMO-targeting of cellular proteins on HIV replication, supernatants harvested from PMO-treated and control THP and MT-2 cells were assayed using an HIV p24 antigen ELISA. The rationale behind this assay is described in the following paragraph.

The HIV-1 gag protein p24 is the major internal structural component of the virion core. The Coulter p24 assay is an enzyme-liked immunosorbent assay (ELISA) performed using a commercially available kit (Beckman Coulter). It was developed specifically for the detection and quantitation of HIV-1 p24 in plasma or serum, for clinical purposes, or in tissue culture supernatants, to monitor virus replication. Each kit contains a 96-well microtiter tray pre-coated with a monoclonal antibody to p24. A specimen of plasma, serum or tissue culture supernatant is added to each well along with a viral lysis buffer and incubated to allow any p24 antigen present to bind to the coated well. Following a wash, biotinylated human anti-HIV IgG is added and incubated to allow complexing to any bound p24. Following another wash, strepatavidin-HRP is added to complex with any bound biotinylated complexes. A substrate reagent is added to form a blue color upon reaction with HRP, the reaction is stopped with acid, and the absorbance measured spectrophotometrically. The intensity of color development (optical density at 450 nm) is directly proportional to the amount of p24 present in the test sample. For each assay, a series of wells are devoted to running a standard curve that utilizies known amounts of purified p24 antigen. The standard curve is used to monitor assay performance and to qualitatively determine the amount of p24 (in pg/ml) in the test sample (optical density at 450 nm was converted to pg/ml p24 using a standard curve and plotted on the Y-axis).

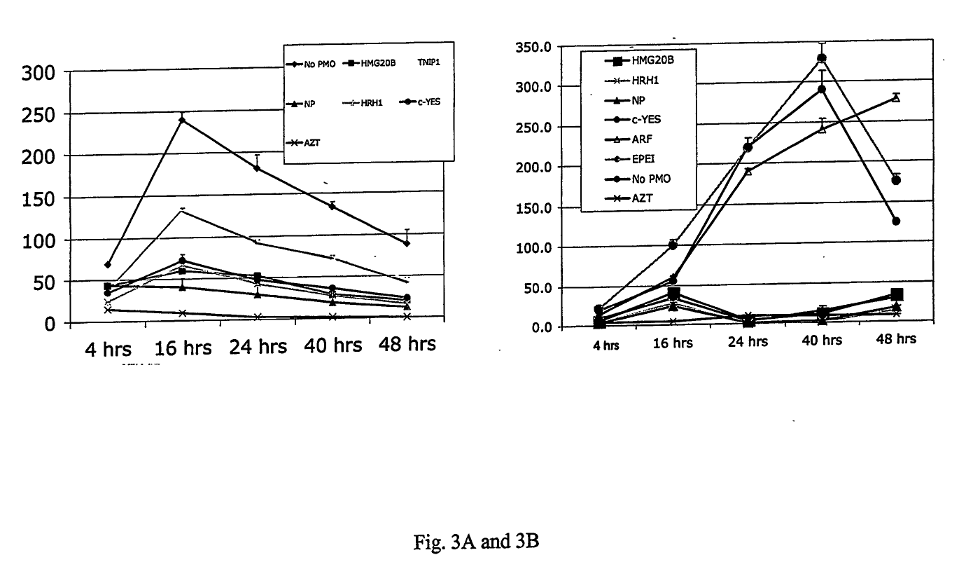

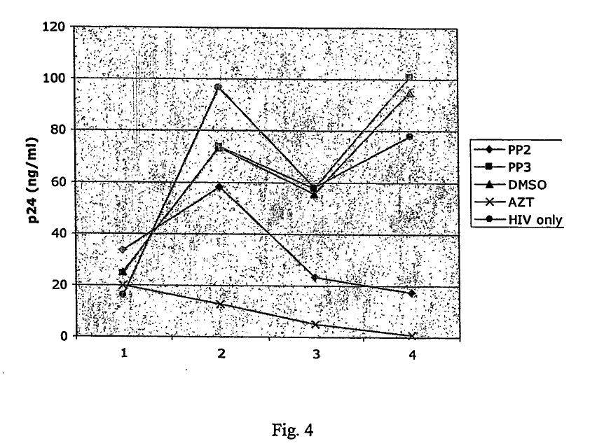

The levels of p24 measured in supernatants from PMO-treated and control THP-1 and MT-2 cells were plotted graphically as p24 production (Y-axis) versus time (hours post-infection; hpi) along the X-axis. Typical results for THP-1 cells and MT-2 cells are graphically depicted in FIG. 3 A and B respectively.

FIG. 3A shows inhibition curves of HIV replication in HIV-infected THP-1 cells human myeloid (monocyte/macrophage) cell line derived from an acute monocytic leukemia) by PMOs specific for particular HIV-induced cellular genes as follows: upper filled diamonds correspond to no PMO control; triangles correspond to TNIP; lower filled diamonds correspond to c-YES; dark “X”s correspond to HRH1; light “X”s correspond to NP; filled squares correspond to HMG20; and vertical lines correspond to AZT control. The HIV-1 P24 ELISA assay monitors HIV p24 gag production (in pg/ml along the y-axis) by PMO-treated HIV-infected THP-1 cells (optical density at 450 nm was converted to pg/ml p24 using a standard curve and plotted on the Y-axis). The X-axis shows time (“T1-T4” typically correspond to 4, 16, 24, 40 and 48 hrs PI). PMOs corresponding to HMG20, HRH-1, NP and c-YES were particularly effective at reproducibly inhibiting HIV replication.

FIG. 3B shows inhibition curves of HIV replication in HIV-infected MT-2 cells (human T cell leukemia cell line) by PMOs specific for particular HIV-induced cellular genes as follows: upper curve filled diamonds correspond to EPEI (ethoxylated polyethylenimine); open squares correspond to HIV only; open triangles correspond to ARF; filled triangles correspond to NP; lower curve filled diamonds correspond to HMG20; “X”s correspond to c-YES; filled squares correspond to HRH-1; and vertical lines correspond to HIV plus AZT control. The HIV-1 P24 ELISA assay monitors HIV p24 gag production (in pg/ml along the y-axis) by PMO-treated HIV-infected MT-2 cells (optical density at 450 nm was converted to pg/ml p24 using a standard curve and plotted on the Y-axis). The x-axis shows time (“T1-T4” typically correspond to 4, 16, 24, 40 and 48 hrs PI). As in the case of HIV-infected THP-1 cells (FIG. 3A), PMOs corresponding to HMG20, HRH-1, NP and c-YES were particularly effective at reproducibly inhibiting HIV replication.

Systematic testing of PMOs in this system led to the identification of 4 PMOs that reproducibly inhibited HIV replication in both the monocyte and the T cell line. These PMOs were designed to inhibit expression of the following cellular proteins: HMG20, HRH-1, NP and c-Yes.

TABLE 2 shows the validation results for five induced genes identified in the experiments of EXAMPLE 2 herein above. For four of the induced genes, suppression by sequence-specific PMO antisense oligonucleotides led to substantial inhibitory effects (either full or intermediate inhibition) on HIV replication, as measured by p24 gag production: HMG20B (homo sapiens high-mobility group 20B, accession number NM_{—}006339, and known variants); HRH1 (homo sapiens histamine receptor H1, accession numbers NM_{—}00861 and BC060802, and known variants); NP (homo sapiens nucleoside phosphorylase, accession number NM 000270, and known variants); and YES1 (homo sapiens v-yes-1 Yamaguchi sarcoma viral oncogene homolog 1, accession number NM_{—}005433, and known variants). The specific PMO antisense oligomers used in these experiments for silencing the HIV-induced gene sequences are also shown in TABLE 2, along with corresponding SEQ ID NOS.

TABLE 2

Validated Gene Targets; suppression
(silencing) of particular HIV-1 89.6-
induced genes substantially inhibited HIV
replication as measured by gag 24
production.

Extent of PMO-

| | |
|---|---|
| PMO Antisense Sequence | induced Inhibition |

| | | |
|---|---|---|
| GENE | (5′ to 3′) | of HIV replication |

| | | |
|---|---|---|
| HMG20B | CGCCCAGCATCTTGGTGATCTCGGG | positive |
| HRH1 | GCGAAAGAGCAGCCGCCAGTTATGG | positive |
| NP | CTTCATAGGTGTATCCGTTCTCCAT | positive |
| YES | TTTCTTTACTTTTAATGCAGCCCAT | positive |
| ARF1 | ATGCTTGTGGACAGGTGGAAGGACA | (negative) |

TABLE 3 summarizes GenBank mRNA and EST accession numbers for particular HIV-induced genes, including for the four validated gene sequences listed in TABLE 2. Gene names, Unigene clusters, and GenBank accession numbers are as assigned by the National Center for Biotechnology Information (NCBI), and are incorporated by reference herein, including splice and allelic variants of mRNA sequences.

TABLE 3
GenBank accession numbers for particular HIV-induced genes, including
for the HMG20B, HRH1, NP, and YES1 gene sequences validated herein.

| | Unigene | Accession Numbers; | Accession Numbers; |
|---|---|---|---|
| GENE | Cluster | mRNAs | ESTs |
| HMG20B | Hs.406534 | AF072836.1, NM_006339.1, AL355709.1, | BE379548.1, BG768730.1, BG768163.1, |
| | | AL355691.1, AL355703.1, AF146223.1, | BG768132.1, BG767749.1, BE387852.1, |
| | | AF072165.1, AF288679.1, BC003505.1, | BE387970.1, BE388514.1, BE388977.1, |
| | | BC002552.1, AF331191.1, BC004408.1, | BG767740.1 |
| | | BC021585.1, AK090733.1, AF318366.1, | |
| | | AL355698.1, AL355702.1 | |
| HRH1 | Hs.1570 | NM_000861.2, D28481.1, AY136743.1, | CB960117.1, BI333356.1, AI925675.1, |
| | | Z34897.1, AF026261.1 | AI565677.1, AI952059.1, AI889049.1, |
| | | | AI926322.1, AA613545.1, AA582697.1, |
| | | | AI954670.1 |
| NP | Hs.75514 | NM_000270.1, AK098544.1, AF116670.1, | BG032220.1, BG177426.1, BG429168.1, |
| | | X00737.1 | BG500365.1, BG527082.1, BG831409.1, |
| | | | BI087530.1, BI225123.1, B1225577.1, |
| | | | BI197180.1 |
| YES | Hs.194148 | BC048960.1, NM_005433.2, BC031080.1, | BQ932574.1, BI861691.1, BI560649.1, |
| | | M15990.1 | BI548150.1, AI799358.1, AI367561.1, |
| | | | AA604737.1, AI445609.1, R28423.1, |
| | | | R25397.1 |

HIV-induced genes excluded as therapeutic targets by PMO antisense validation protocol. The above Examples show that with respect to particular identified HIV-induced genes (e.g., ARF1), treatment of HIV-infected MT-2 with the respective antisense PMO oligonucleotides had little or no affect on HIV replication (p24 gag production) despite effectiveness of such antisense agents in mediating silencing of the respective gene sequences. This was not unexpected, because HIV-related modulation of some cellular genes would reasonably be expected to be either ancillary to, or downstream from the regulatory cascades involved in HIV replication.

Significantly, the identification of HIV-induced gene sequences which, upon silencing, have no effect on HIV replication provides internal confirmation (apart from the use of particular control PMO antisense molecules, etc.) that the inventive gene-silencing mediated inhibition of HIV replication is not mediated through ancillary or non-sequence-specific secondary effects of the respective PMO antisense molecules.

Therefore, data presented herein describes, teaches and supports the use of sequence-specific PMO antisense oligomers, inter alia, for (i) validation of therapeutic ‘targets’; that is, for identification of HIV-induced cellular gene products required for HIV-induced cellular phenomena (e.g. replication, etc.), and (ii) as effective, non-toxic inhibitors of such validated therapeutic targets for modulation of HIV infection and treatment of HIV-related disorders and diseases. This utility is especially valuable where the particular gene products otherwise lack suitable art-recognized small molecule inhibitors.

Additionally, in view of deficiencies in the prior art teachings, these data emphasize the significance of functional validation of HIV-induced gene sequences, according to the present invention to provide targets, compositions and methods having utility for blocking HIV infection and replication, and for treating HIV-related conditions and diseases.

### EXAMPLE 4

### HIV Replication in MT-2 Cells and THP-1 Cells was Inhibited by the src Family Kinase Inhibitor PP2, and c-Yes-Specific siRNA, Respectively

As discussed herein, particular embodiments of the present invention provide screening assays for identification of compounds useful to modulate HIV infection, comprising: contacting HIV-infected cells with a test agent; measuring, using a suitable assay, expression of at least one validated HIV-induced cellular gene sequence; and determining whether the test agent inhibits said validated gene expression relative to control cells not contacted with the test agent, whereby agents that inhibit said validated gene expression are identified as compounds useful to modulate HIV infection and/or replication.

Preferably, the at least one validated HIV-induced cellular gene sequence is selected from the cDNA and protein sequence group consisting of HMG20B, HRH1, NP and c-YES, and combinations thereof (i.e., consisting of SEQ ID NOS:1-9). Preferably, expression of at least one validated HIV-induced cellular gene sequence is expression of mRNA, or expression of the protein encoded thereby. Preferably, agents that inhibit said validated gene expression are further tested for the ability to modulate HIV-mediated effects on cellular proliferation and/or phenotype.

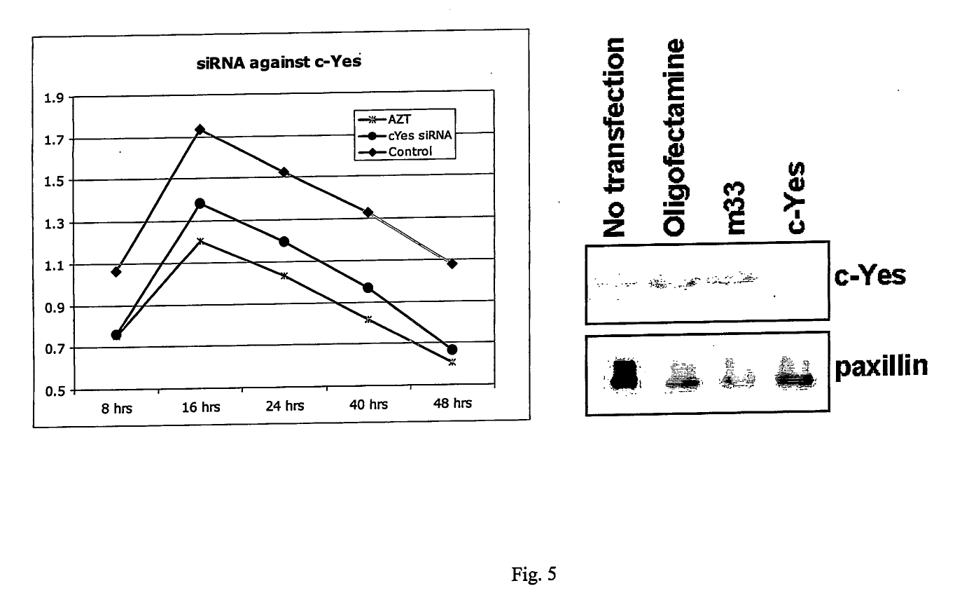

As shown in FIG. 4, addition of the src family kinase inhibitor PP2 to HIV-infected MT-2 cells resulted in substantial decrease in HIV p24 production. Specifically, FIG. 4 shows inhibition of HIV p24 production in MT-2 cells infected with HIV 89.6 in the continued presence (10 μM) of the src family kinase inhibitor PP2 (4-Amino-5-(4-chlorophenyl)-7-(t-butyl)pyrazolo[3,4-d]pyrimidine; e.g., Calbiochem; catalog no. 529573). PP3, and DMSO correspond to inactive analog, and carrier control, respectively. AZT inhibition is also included as a positive control. Time 0 is immediately post-infection, whereas times 1, 2 and 3 correspond to 24, 48, and 72 hrs PI, respectively, along the X-axis. The curves are as follows: upper closed diamonds correspond to HIV alone; lower curve closed diamonds correspond to PP2; filled squares correspond to PP3; filled triangles correspond to DMSO; and “X”s correspond to AZT. The HIV-1 P24 ELISA assay monitors HIV p24 gag production (in pg/ml along the Y-axis) by the various treated HIV-infected MT-2 cells (optical density at 450 nm was converted to pg/ml p24 using a standard curve and plotted on the Y-axis).

Likewise, in particular embodiments, other small molecule modulators of HIV replication are identified by the inventive screening assays.

HIV-infected THP-1 cells were treated with c-YES-specific siRNA to further validate the c-YES target. Methods of preparing and using siRNA are well known in the art, and are generally disclosed in U.S. Pat. No. 6,506,559, incorporated herein by reference (see also reviews by Milhavet et al., Pharmacological Reviews 55:629-648, 2003; and Gitlin et al., J. Virol. 77:7159-7165, 2003; incorporated herein by reference).

Briefly, THP-1 cells (5×10⁵ cells/test) were transfected with siRNA at 8 μl (20 μM) siRNA per cell sample using Oligofectamine™ reagent and a standard lipofection protocol. Cells were infected the next day (20 hrs later) with HIV-1 89.6 (50 μl concentrated virus per cell sample). Four (4) hrs later, virus inoculums were removed and cells rinsed (time 0). Supernatants were harvested for p24 ELISA at 8, 16, 24, 40 and 48 hrs post infection (hpi).

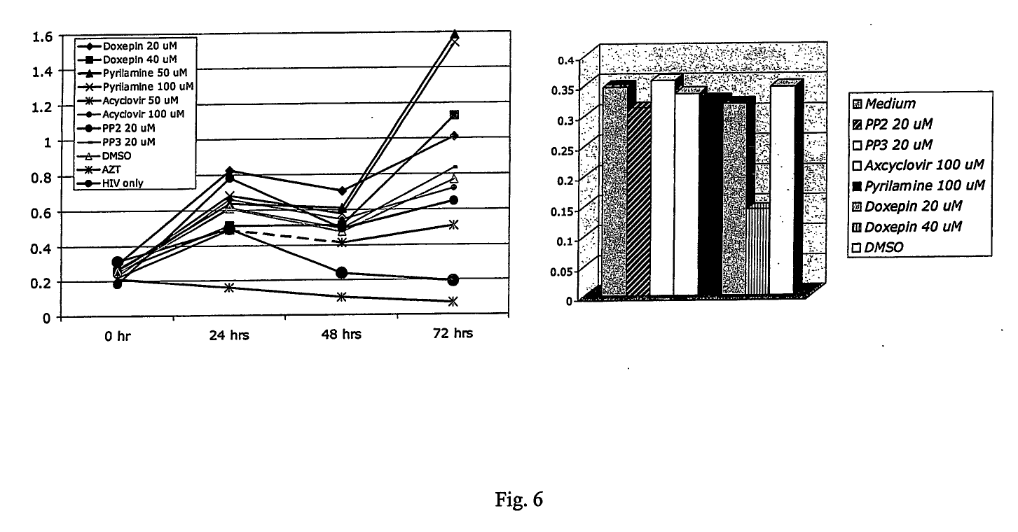

FIG. 5 shows that siRNA molecules against (specific for) c-Yes inhibited HIV replication. The left panel shows p24 ELISA assay results of HIV infected THP-1 cells treated with siRNA (316 c-Yes siRNA, from Dharmacon, Lafayette, Colo.). Time post-infection is plotted on the x-axis, and optical density at 450 nm (a measure of p24 production as described herein above) is plotted on the y-axis. The right panel shows a Western blot analysis of a SDS-PAGE gel, using antibodies to c-Yes and paxillin (as a cellular control). For this analysis, Huh7 cells were transfected with: c-Yes-specific siRNA (rightmost lane), m33 (negative control; second lane from right), transfection reagent only (Oligofectamine™; third lane from right), or left untreated (no transfectin; leftmost lane).

The ability to substantially reduce HIV replication through specific inhibition of src family kinase c-YES activity in MT-2 and THP-1 cells further demonstrates a critical role for src family kinase signaling in HIV replication in vivo, and further supports a role for upregulation of c-YES as a factor in HIV replication and related events in vivo.

### EXAMPLE 5

### Compounds that Inhibit Validated HIV-Induced Cellular Targets were Tested for Anti-HIV Activity and Toxicity

As discussed herein, particular embodiments of the present invention provide screening assays for identification of compounds useful to modulate virus (e.g., HIV) infection, comprising: contacting virus-infected cells with a test agent; measuring, using a suitable assay, expression of at least one validated virally-induced cellular gene sequence; and determining whether the test agent inhibits said validated gene expression relative to control cells not contacted with the test agent, whereby agents that inhibit said validated gene expression are identified as compounds useful to modulate HIV infection and/or replication. Preferably, the compounds are further tested to show that such inhibition of induced cellular gene expression/activity is reflected in inhibition of, e.g., viral infection, replication, maturation or progression (validation). Preferably, compounds that are effective in inhibiting validated targets, are further tested for toxicity at concentration ranges effective to inhibit the validated targets, and e.g., viral infection, replication, maturation or progression.

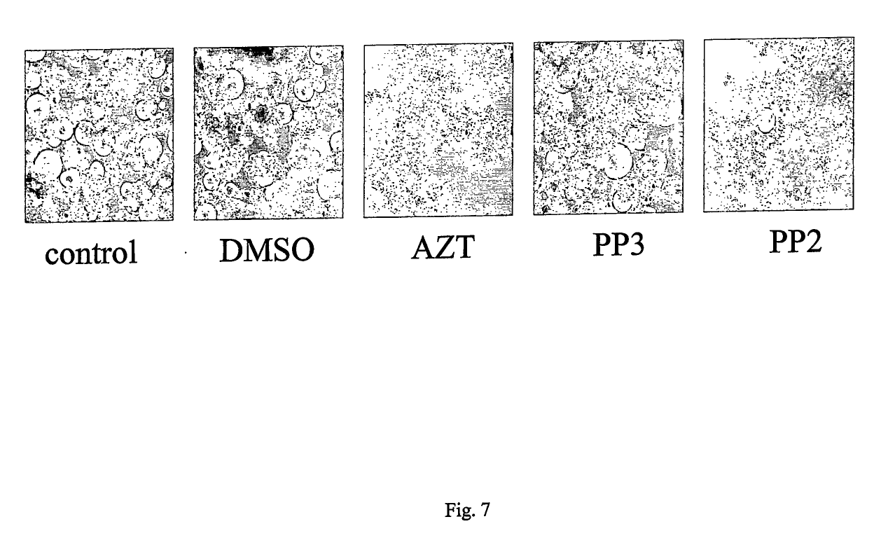

FIG. 6 (left panel) shows testing of compounds that inhibit validated targets (e.g., NP, HRH1, c-Yes) for anti-HIV activity. In this analysis, p24 ELISA assays were used to measure p24 production in HIV-infected cells treated with various compounds: filled diamonds, 20 μM Doxepin; filled squares, 40 μM Doxepin; filled triangles, 50 μM Pyrilamine; “×”, 100 μM Pyrilamine; dark×plus vertical line, 50 μM Acyclovir; small filled circles, 100 μM Acyclovir; light large filled circles, 20 μM PP2; “−”, 20 μM PP3; open triangles, DMSO; light×plus vertical line, AZT; and dark large filled circles, HIV only (control). The SFK inhibitor PP2 resulted in substantial inhibition of HIV.

The right panel of FIG. 6 shows use of the art-recognized XTT respiration assays to assess compound toxicity. The agents tested are cross-referenced to the respective bar graphs as shown in insert of the right panel. Except for the higher concentration of doxepin, the compounds were not toxic at the concentrations used. Briefly, the XTT assay is based on the cleavage of XTT (sodium 3-[1-(phenylamino-carbonyl)-3,4-tetrazolium]-bis (4-methoxy-6-nitro) benzene sulfonic acid hydrate) to form formazan by mitochondrial respiration in viable cells (see, e.g., Marshall et al., Growth Regul. 5:69-84, 1995).

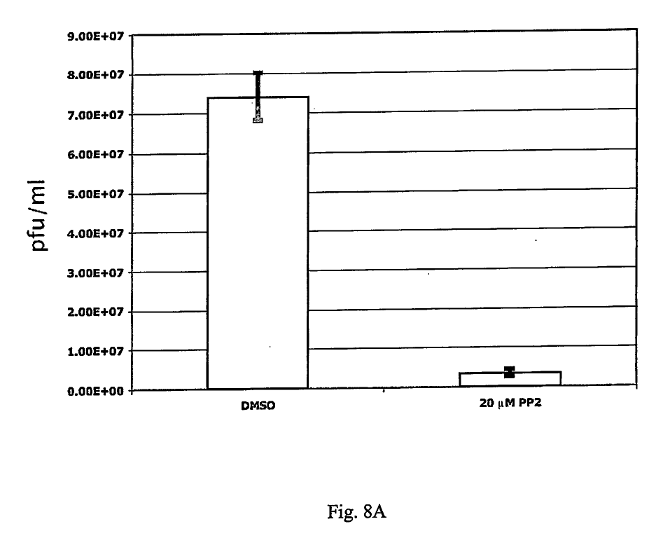

FIG. 7 shows that treatment of HIV-infected MT-2 cells with AZT and PP2 resulted in reduced sysncytia formation. The panels of FIG. 7 show the relative extent of HIV-induced syncytia formation in MT-2 cells at 48 hours post infection under various conditions. “Control” cells are HIV-infected cells, but are not treated with any test agent. “DMSO” treatment alone is also included as a control. Other treatments include, AZT, PP3 and PP2, all solubilized in DMSO. Treatments with AZT and PP2 resulted in substantially reduced syncytia formation, reflecting inhibition of HIV replication within the infected MT-2 cells.

### EXAMPLE 6

### Methods and Compositions for Inhibition of flavivirus Infection

According to another aspect of the present invention, the human hepatocellular carcinoma derived cell line Huh7 supports replication of flaviviruses, including West Nile virus (WNV), Japanese encephalitis virus (JEV), yellow fever virus (YFV) and Dengue fever virus (DEN), and was used herein as a host cell model for particular embodiments. Additionally, SKN-MC neuroblastoma cells were used for particular embodiments.

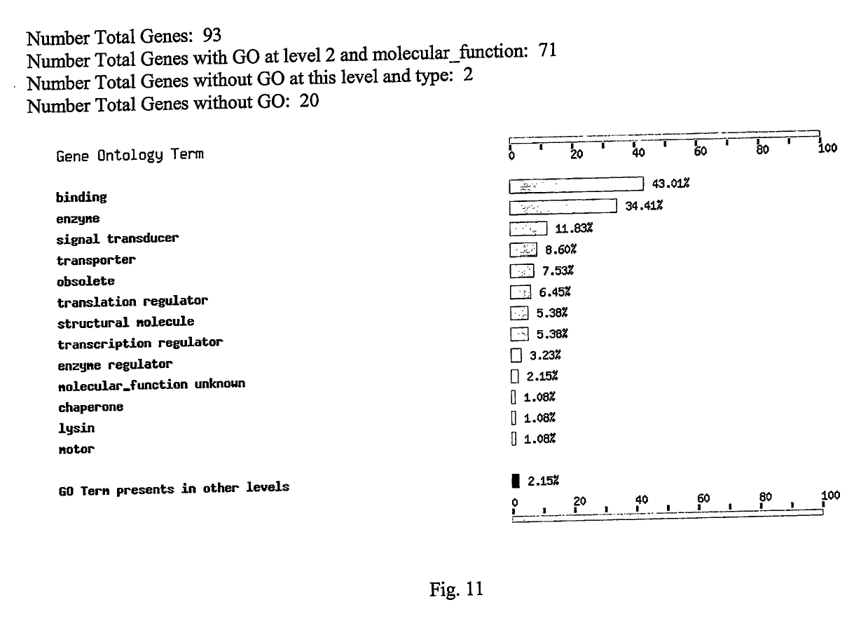

Gene microarrays, as described herein above in relation to HIV-infected cells, were used to define the general host cell response to flavivirus infection. Specifically, gene microarrays comprising representations of 8,100 human genes were used to compare host cell gene expression following infection with WNV, JEV, YFV and DEN. For WNV, K-means clusters corresponding to 238 genes having changes in expression at any one time point post-WNV infection are shown in FIG. 10.

Likewise, microarray results with JEV showed greater than 100 host genes whose expression was increased by between 2- and 10-fold at 15 h post-infection (hpi). Additionally, quantitative RT-PCR (Taqman) was used to show that several of these genes are also up-regulated in Huh7 cells during WNV infection (e.g., at 15 hpi).

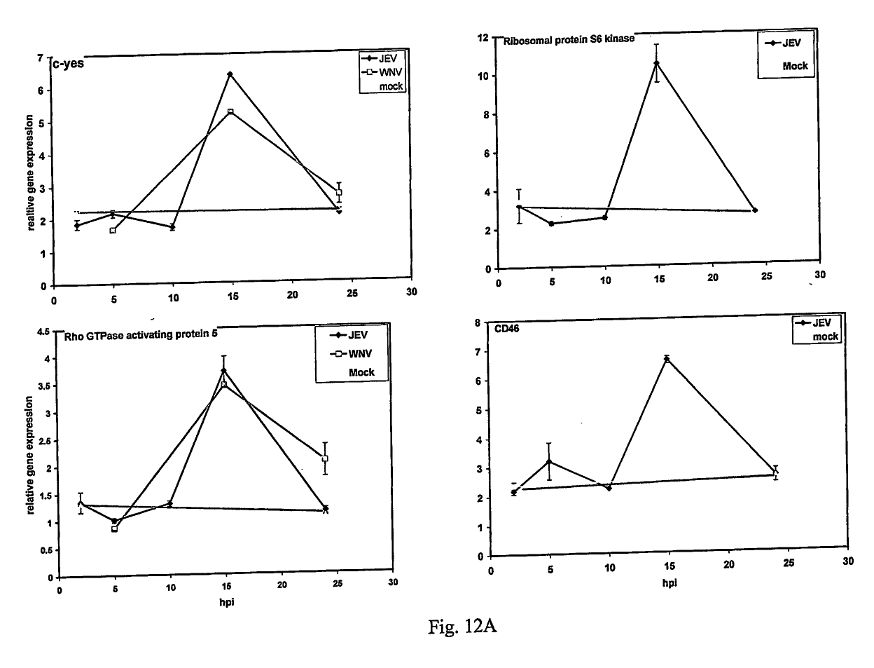

FIG. 11 shows a classification by function of genes up-regulated at 15 h post-infection (hpi) in West Nile Virus. Analysis of the up-regulated genes revealed that they include components of a signal transduction cascade that signals through a member of the src-family tyrosine kinase family (FIGS. 4 and 5).

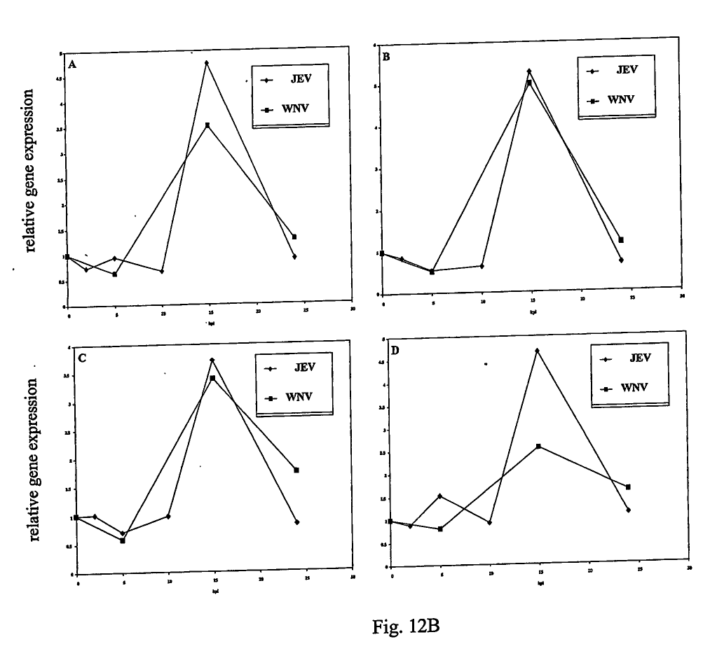

FIGS. 12A and 12B show plots of the observed relative gene expression, for exemplary up-regulated genes (c-Yes, “A”; ribosomal protein S6 kinase, “B”; Rho GTPase activating protein 5, “C”; and CD46, “D”) of the genes reported to be upregulated at 15 h by the spotted array experiment are confirmed by RT-PCR analysis, at various times (hours) post-infection with Japanese encephalitis virus (JEV; filled diamond symbols), West Nile virus (WNV; filled square symbols) and mock infection (light triangles).

Significantly, for example, as shown in EXAMPLE 7 below, the addition of a specific inhibitor of src-family kinases, to Huh7 cells at the time of WNV infection resulted in a dose-dependent reduction of virus recovered from the culture supernatant, as measured by plaque assay and quantitative RT-PCR of viral RNA (up to 90%, as compared to control samples). Inhibition of src-family kinase, however, did not result in a corresponding reduction of viral RNA found within the infected cells, indicating that this pathway (i.e., src family kinases, and related signal transduction) plays a role in viral assembly or egress from the host cell.

Likewise, src-family kinases can be inhibited by antisense, ribozymes, and siRNA.

### EXAMPLE 7

### src Family Kinase Inhibitors Resulted in a Substantial Decrease of Infectious WNV

DNA Microarray Analysis

Methods. Viral stocks for all microarray infections were grown on Vero cells. Viral titers were determined by limiting dilution on Veros. In order to avoid effects from cytokines present in viral stocks, the virus was concentrated and purified before infection of target cells. Approximately 28 ml of stock was underlayed with 7 ml of 20% sorbitol cushion and spun for 1.5 h at 25,000 rpm (113,000×g) and 20° C. in an SW28 rotor (Beckman). The virus pellet was resuspended in Dulbecco's modified Eagle medium (DMEM) supplemented with 0.1% bovine serum albumin (BSA). Approximately 3×10⁶ Huh7 cells were infected at a multiplicity of 10 with the concentrated virus stock. At 0.5 h, 2 h, 5 h, 10 h, 15 h, 20 h and 26 h post-infection, duplicate plates were collected and RNA isolated by TRIzol reagent (Invitrogen) according to manufacturer's instructions. Labeled cDNA was generated from the total RNA and hybridized to cDNA spotted arrays. The spotted arrays contained cDNAs representing almost 8400 genes. Approximately 200 genes were identified as having increased expression in both JEV and WNV infected Huh7 cells at the 15 hour time point.

Confirmation of microarray results. Quantitative real-time RT-PCR analysis was performed on RNA samples harvested from independent infections of Huh7 cells with JEV or WNV, to confirm the results of the initial microarray experiment. As shown in FIG. 12A or 5B, four (c-Yes, “A”; ribosomal protein S6 kinase, “B”; Rho GTPase activating protein 5, “C”; and CD46, “D”) of the genes reported to be upregulated at 15 h by the spotted array experiment are confirmed by RT-PCR analysis. RT-PCR was performed using Omniscript reverse transcriptase (Qiagen) and random hexamer primers, followed by PCR in an ABI Prism 7700 Sequence Detector using gene specific primers in the presence of SYBR green (Applied Biosystems). Interestingly, and in agreement with the array results, the upregulation seems to be a transient event that occurs at approximately 15 h post-infection (hpi), but is absent by 24 hpi.

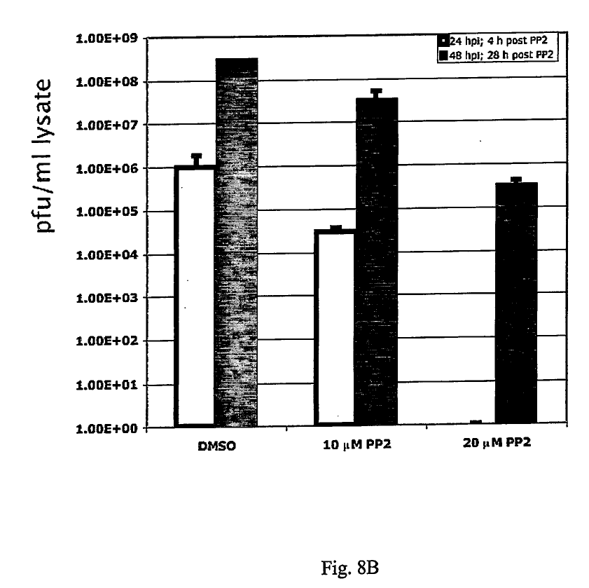

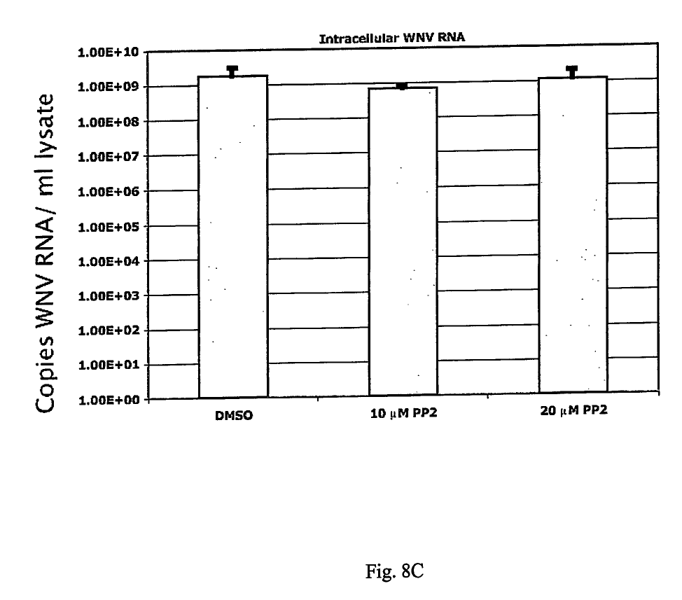

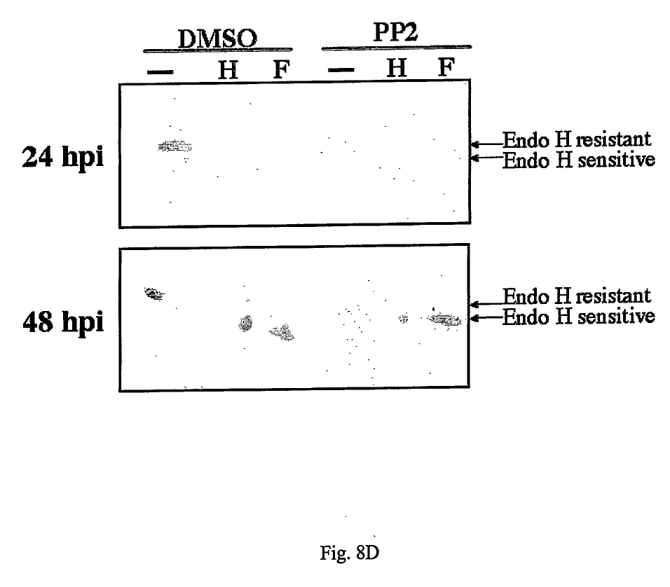

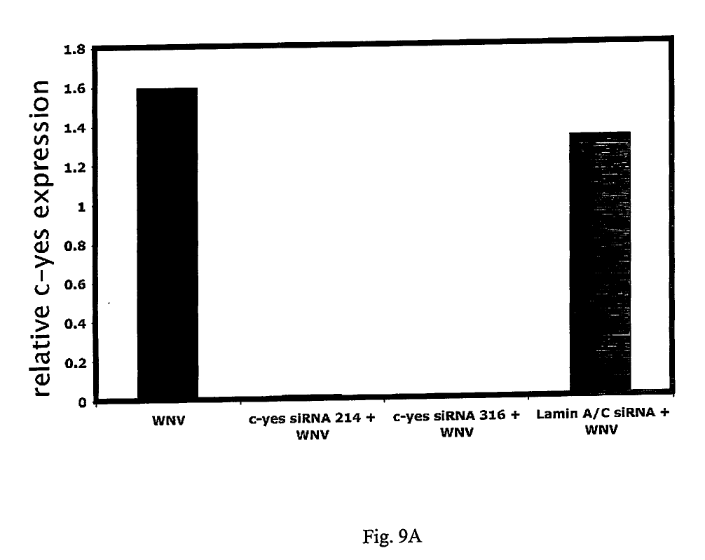

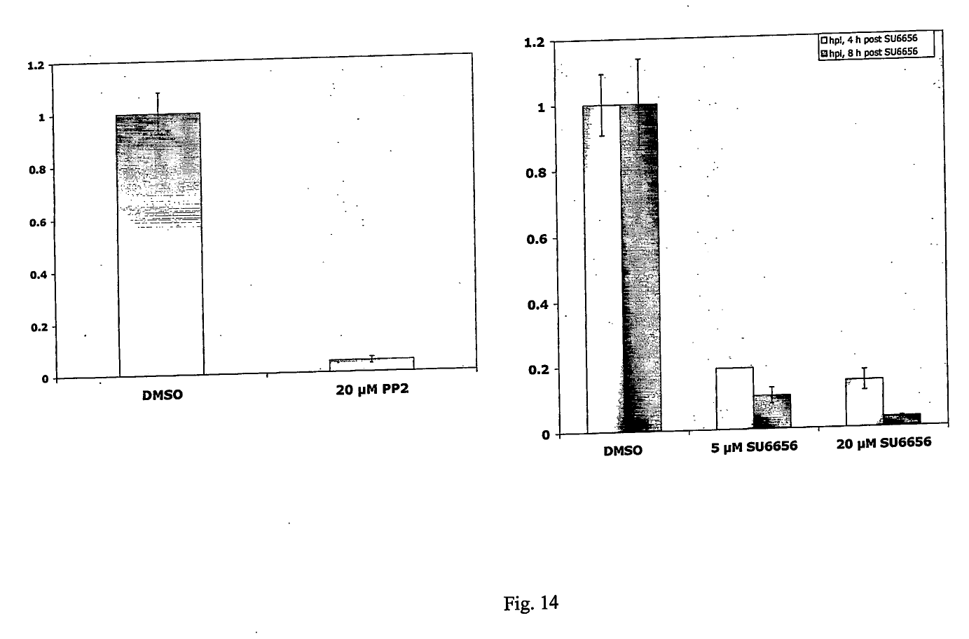

Addition of the src family kinase inhibitor PP2 (4-Amino-5-(4-chlorophenyl)-7-(t-butyl)pyrazolo[3,4-d]pyrimidine; e.g., Calbiochem; catalog no. 529573) at 20 μM to WNV infected SKN-MC neuroblastoma cells (multiplicity of infection of 5) resulted in a 30-fold decrease of infectious WNV in the supernatant 22 hours post-infection (20 h post-PP2 addition) (FIG. 8A, and FIG. 14, left panel). Specifically, FIG. 8A shows that PP2 inhibits accumulation of infectious WNV in cell culture supernatants. SKN-MC neutoblastoma cells were infected with WNV at a multiplicity of 5. Two (2) hours post-infection (hpi, cells were treated with PP2 to a final concentration of 20 μM, or DMSO only as a control. Culture supernatant was harvested 20 hours post-infection, and virus was measured by plaque assay on Vero cells. Likewise, FIG. 14 shows the observed effect of src family tyrosine kinase (SFK) inhibitors on WNV (NY1999) infection in SKN-MC neuroblastoma cells.

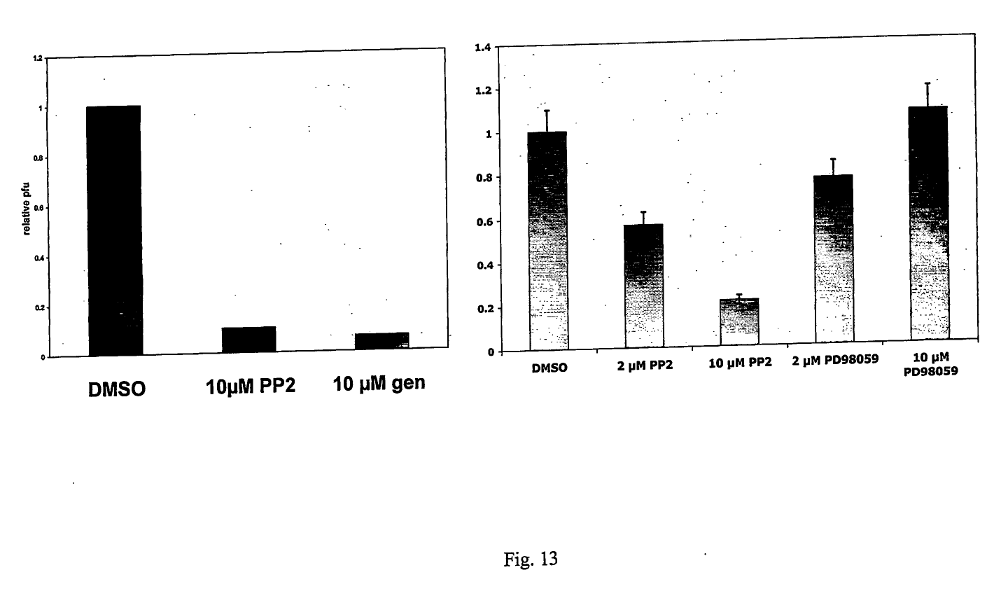

Similar results were obtained using Huh7 cells. FIG. 13 shows the observed effect of tyrosine kinase inhibitors on WNV (NY1999) infection in a human hepatocellular carcinoma-derived cell line (Huh7 cells).

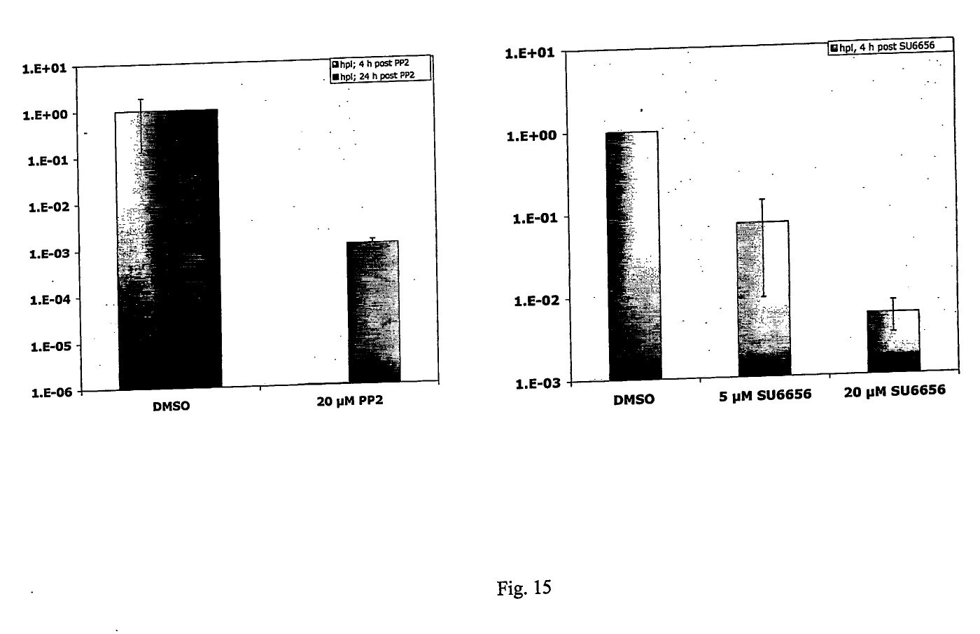

Additionally, infectious virus within infected cell lysates was decreased almost 10⁶-fold four hours post-addition of PP2 (FIG. 8B, gray bars; and FIG. 15, left panel). FIG. 8B shows the effect of PP2 in inhibiting accumulation of infectious virus within infected SKNMC cells. Specifically, SKN-MC cells were infected with WNV as above. At 2 hpi, cells were treated with PP2 at the indicated final concentrations. At 20 hpi, cells were re-fed with media containing fresh PP2 at the same concentration. Cells were washed 3-times with phosphate buffered saline (PBS), resuspended in 250 μl PBS, and then lysed by 3 successive freeze/thaws. Lysates were centrifuged at 13,000×g for 5 minutes, followed by collection of the supernatant. Infectious virus in soluble lysate was measured by plaque assay on Vero cells. Samples were collected 4-hours post-addition of fresh PP2 (gray bars) and 24 hours post addition of fresh PP2 (black bars). 24-hours post-addition of PP2, virus within the infected cell was approximately 500-fold lower, indicating some recovery from the effect of the drug (FIG. 8B, black bars). FIG. 15 shows the amount (pfu/ml) of intracellular infectious WNV after SFK inhibitor treatment in SKN-MC cells.

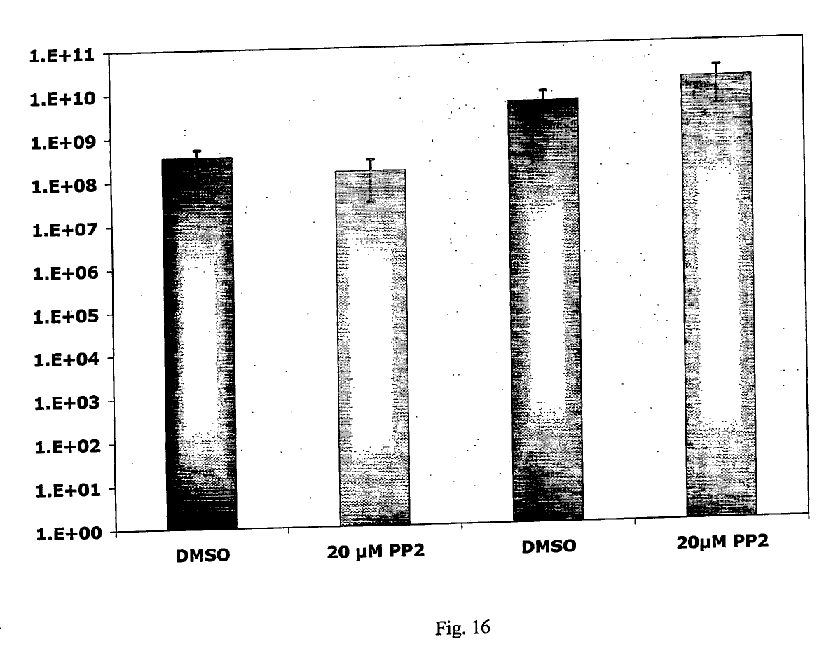

Quantification of viral RNA within infected cells (4 hours post PP2 addition) showed no significant difference between treated or untreated cells (FIG. 8C; and FIG. 16), suggesting that the effect of the inhibitor is exerted at a post-viral RNA replication stage.

FIG. 8C shows that levels of WNV RNA within infected SKN-MC cells did not change with the addition of PP2. Specifically, cells were prepared as described above in relation to FIG. 8B. Four hours post fresh PP2 addition, RNA was isolated from 200 μl of cell lysate by Trizol reagent (Invitrogen), and 1 μg total RNA was used in a quantitative RT-PCR reaction (Taqman) using WNV-specific primer and probe set and standards. An additional RT-PCR was performed using β-actin-specific primers and probe, for an internal control. WNV RNA quantities were normalized to the β-actin values. FIG. 16 shows the amount (pfu/ml) of intracellular WNV RNA following PP2 treatment in SKN-MC cells.

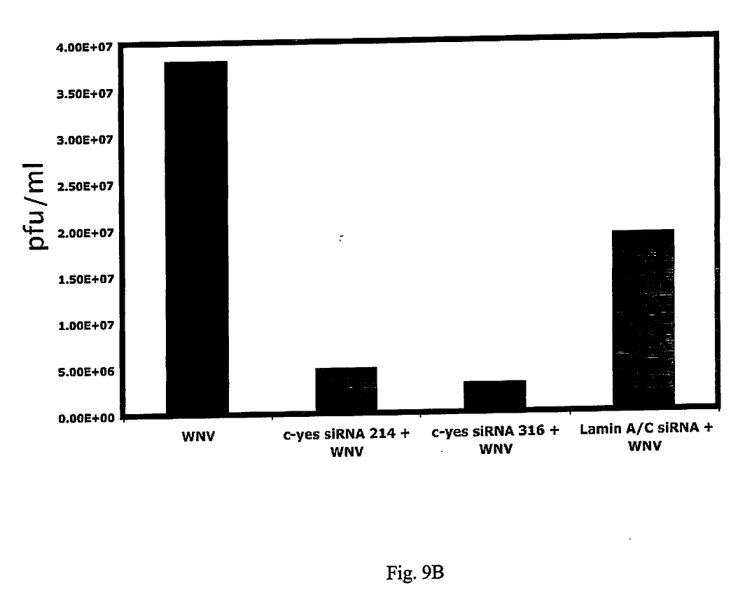

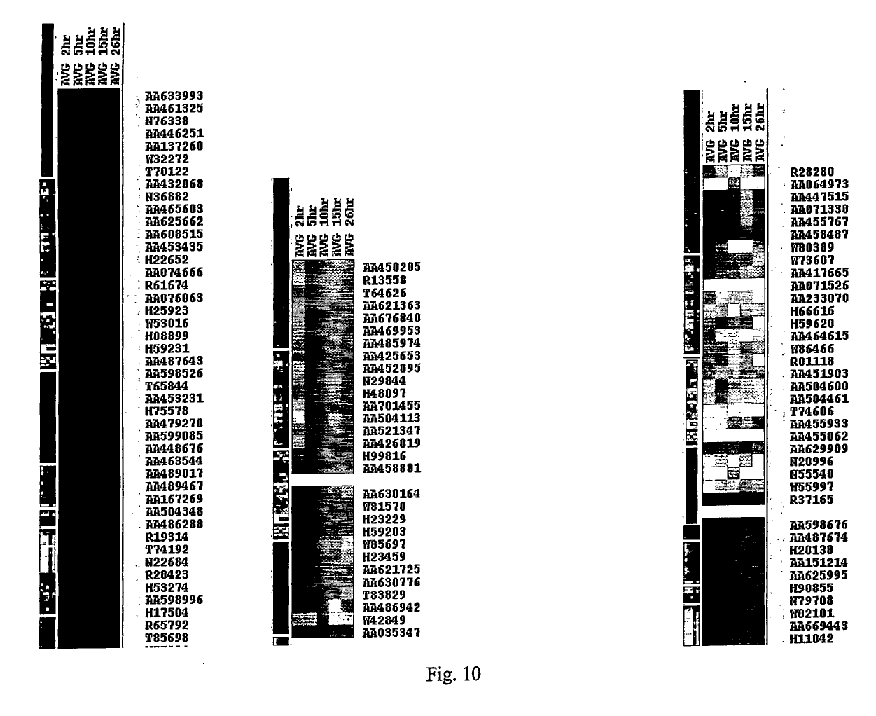

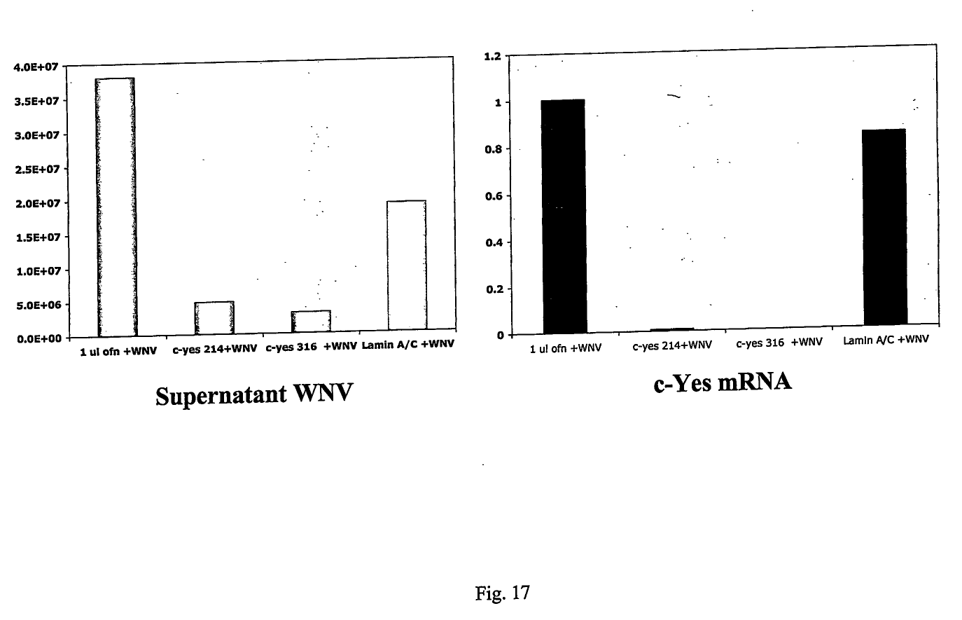

Additionally, in Huh7 hepatocellular carcinoma cells, transfection of siRNA designed to inhibit production of c-yes, but not a control siRNA (lamin A/C) resulted in an approximately 8-fold reduction of the amount of WNV in the culture supernatant (FIGS. 9A and 9B; and FIG. 17).

Specifically, FIG. 9A shows the observed reduction of c-yes mRNA in response to c-yes-specific siRNA. Huh7 cells were transfected with either of c-yes-specific siRNAs 214 or 316, or a control siRNA directed against Lamins A and C. Transfection of 300,000 cells was carried out with 2 μl of a 20 μM siRNA (Dharmacon) and 1 μl Oligofectamine (Invitrogen) according to the manufacturer's protocol. Cells were transfected 24 hours post-transfection, and cells and supernatant harvested at 24 hpi. 1 μg total cellular RNA was used for quantitative RT-PCR with c-yes-specific primers.

FIG. 9B shows the measured levels of WNV in the siRNA-treated cultures. Culture supernatant from Huh7 cells transfected with c-yes specific siRNAs (above) were harvested 24 hpi, and virus was measured by plaque assay on Vero cells.

FIG. 17 shows that C-yes-specific siRNA inhibits c-YES mRNA levels, as well as WNV replication in Huh7 cells (24 hpi; 72 h post siRNA transfection). Supernatant WNV is shown in the left panel, whereas corresponding c-YES mRNA levels are shown in the right panel.

These results identify and validate c-yes as a src-family kinase therapeutic target for the treatment of flavivirus and related conditions, and indicates that PP2 may exert its effect on flavivirus infection, at least in part, through this target.

At 24 hours post-addition of PP2 (FIG. 9B; and FIG. 15), intracellular levels of WNV have recovered somewhat relative to untreated cells.

According to the present invention, inhibitors with longer effective half-lives have a longer period of WNV inhibition.

### EXAMPLE 8

### Inhibition of Src-Family Kinase Activity Prevented Maturation of the WNV E Protein

Inhibition of src-family kinases (SFKs) may result in mis-localization or aberrant trafficking of WNV proteins, thereby interfering with formation of infectious particles. To test this hypothesis, lysates from WNV-infected cells (with or without PP2) were treated with Endoglycosidase H (Endo H) or Peptide N-glycosidase F (PNGase F).

Endo H removes immature high-mannose glycans from proteins. High-mannose structures are added to proteins in the endoplasmic reticulum (ER). Such structures are trimmed and further modified in the Golgi, conferring resistance to endo H. Sensitivity to endo H is therefore indicative of proteins that have not advanced beyond the endoplasmic reticulum in the secretory pathway (Maley, et al., Anal. Biochem. 180:195, 1989.

PNGase F will remove all glycan moieties regardless of modification.

Methods. Vero cells were infected with WNV (multiplicity=3) and treated with PP2 (or DMSO as a control) 1 h post-infection (hpi). At 24 or 48 hpi, cells were lysed in buffer containing 1% NP-40. Lysates were denatured and treated with endo H or PNGase F for 1 h. Samples were resolved by SDS-PAGE, and WNV E was detected by western blotting.

Results. As shown in FIG. 8D, treatment of control cell lysates with endo H (lanes “H”) revealed the presence of roughly equivalent populations of resistant protein (equal in mobility to untreated samples; lanes “−” samples) and sensitive protein (equal in mobility to PNGase F digested proteins; lanes “F”). This ratio is also found at 48 h post infection.

In contrast, in PP2-treated samples, the proportion of endo H-resistant population is dramatically decreased. Again, this result is consistent at 48 h.

Without being bound by mechanism, these data indicate that in the absence of SFK activity (e.g., in the presence of PP2), transit of the E-protein to post-ER compartments of the secretory pathway is impaired, whereby inhibition of SFK activity results in the inhibition of the production of infectious virus; namely, where such transit is required for assembly and maturation of the virus.

1. A method for the treatment of a Flaviviridae virus infection or a human immunodeficiency virus (HIV) infection, comprising administration, to a subject in need thereof, of a therapeutically effective amount of an inhibitor of a src family kinase, whereby the Flaviviridae virus infection or human immunodeficiency virus (HIV) infection is diminished relative to a non-treated subject.

2. The method of claim 1, wherein the Flaviviridae virus is selected from the group consisting of a flaviviruses and hepatitis C virus (HCV).

3. The method of claim 2, wherein the flavivirus is selected from the group consisting of West Nile virus (WNV), Japanese encephalitis virus (JEV), yellow fever virus (YFV), and Dengue fever virus (DEN).

4. The method of claim 2, wherein the Flaviviridae virus is hepatitis C virus (HCV).

5. The method of claim 1, wherein the src family kinase is c-yes kinase.

6. The method of claim 1, wherein the inhibitor comprises a src family kinase-specific antisense oligonucleotide.

7. The method of claim 6, wherein the antisense oligonucleotide is a phosphorodiamidate morpholino oligomer (PMO).

8. The method of claim 1, wherein the inhibitor comprises src family kinase-specific siRNA.

9. The method of claim 1, wherein the inhibitor comprises a small molecule inhibitor of a src family kinase, wherein the small molecule inhibitor is a molecule, or a pharmaceutically acceptable salt thereof, selected from the Formula group consisting of Formula I, Formula I(b), Formula II, Formula III, Formula IV and Formula V: 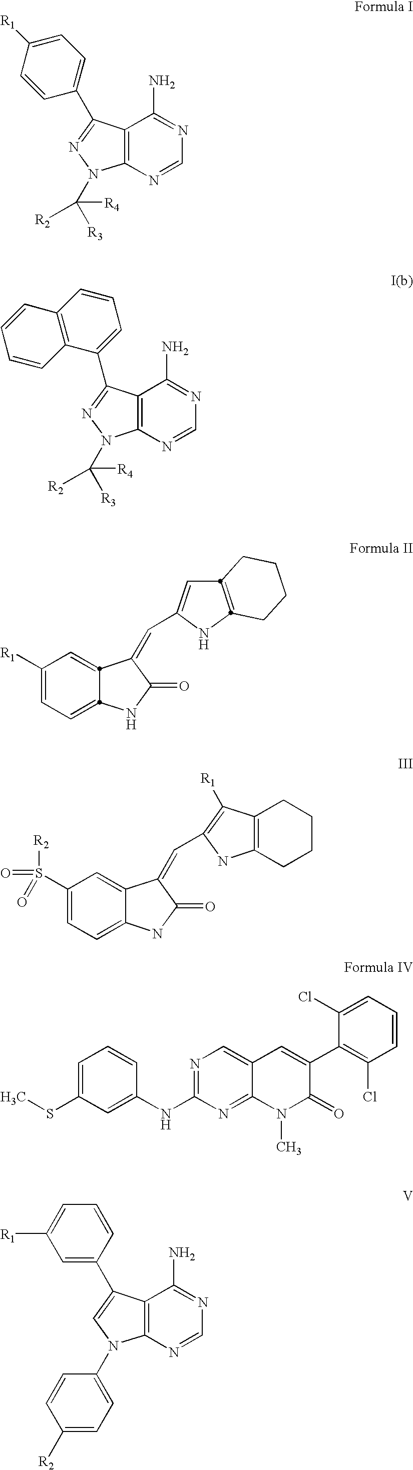
wherein for Formula I or I(b), R₁ is halogen or methyl, and R₂, R₃ and R₄ are independently a C1-C3 straight or branched alkyl; wherein for Formula II, R₁ is —SO₂N(CH₃)₂, or —SO₂NH₂; wherein for Formula III, R₂ is C₂H₅ or NHR₃, wherein R₃ is a C1 to C3 linear or branched alkyl moiety, and wherein R₁ is independently —(CH₂)₃N(CH₃)₂, —CH₂N(CH₂CH₂)₂O, —(CH₂)₂N(CH₂CH₂)₂O, —(CH₂)₃N(CH₂CH₂)₂O, or —(CH₂)₃N(CH₂CH₂)₂N CH₃; and wherein for Formula V, R₁ is either H or —OCH₃, wherein R₂ is independently —(CH₂)₂OH, —CH₂COOH, —(CH₂)₂N(CH₃)₂, —CH₂)₂NH(CH₂)₂OH, —(CH₂)₂NCH₃(CH₂)₂OCH₃, —CH₂)₂N(CH₂CH₂)₂NCH₃, or —(CH₂)₂N(CH₂CH₂)₂CHOH.

10. The method of claim 9, wherein according to Formula I, the small molecule inhibitor is 4-Amino-5-(4-chlorophenyl)-7-(t-butyl)pyrazolo[3,4-d]pyrimidine (“PP2”).

11. The method of claim 9, wherein according to Formula I(b), the small molecule inhibitor is 4-Amino-1-tert-butyl-3-(1′-naphthyl)pyrazolo[3,4-d]pyrimidine.

12. The method of claim 9, wherein according to Formula II, the small molecule inhibitor is 2-oxo-3-(4,5,6,7-tetrahydro-1H-indol-2-ylmethylene)-2,3-dihydro-1H-indole-5-sulfonic acid dimethylamide.

13. The method of claim 9, wherein according to Formula III, R₁ is: —(CH₂)₃N(CH₃)₂; —(CH₂)₃N(CH₂CH₂)₂O; or —(CH₂)₃N(CH₂CH₂)₂NCH₃.

14. The method of claim 9, wherein according to Formula III, R₂ is NH CH₃ and R₁ is —(CH₂)₃N(CH₃)₂, wherein the small molecule inhibitor is 3-[3-(3-dimethylamino-propyl)-4,5,6,7-tetrahydro-1H-indol-2-ylmethylene]-2-oxo-2,3-dyhydro-1H-indole-5-sulphonic acid methylamide.

15. The method of claim 9, wherein according to Formula III, R₂ is C₂H₅, and R₁ is —(CH₂)₃N(CH₃)₂.

16. The method of claim 9, wherein according to Formula III, R₂ is NH CH₃ and R₁ is —(CH₂)₃N(CH₂CH₂)₂O.

17. The method of claim 9, wherein according to Formula III, R₂ is NH CH₃ and R₁ is —(CH₂)₃N(CH₂CH₂)₂N CH₃.

18. The method of claim 9, wherein according to Formula III, R₂ is C₂H₅, and R₁ is —(CH₂)₃N(CH₂CH₂)₂N CH₃.

19. The method of claim 9, wherein according to Formula V, R₁ is —OCH₃R₂, and R₂ is —(CH₂)₂N(CH₂CH₂)₂CHOH.

20-34. (canceled)

35. A method for identification of an agent having therapeutic utility for the treatment of a Flaviviridae virus infection or human immunodeficiency virus infection, comprising:
obtaining cells suitable to support a Flaviviridae virus or a human immunodeficiency virus (HIV) infection;
infecting the cells with the Flaviviridae virus or human immunodeficiency virus;
contacting the infected cells with an agent that inhibits a src family kinase; and
determining whether the Flaviviridae virus infection or human immunodeficiency virus infection is diminished relative to control infected cells not contacted by the agent, thereby identifying the agent as having therapeutic utility for the treatment of the Flaviviridae virus infection or human immunodeficiency virus (HIV) infection.

36. The method of claim 35, wherein the src family kinase is c-yes kinase.

37. The method of claim 35, wherein the Flaviviridae virus is selected from the group consisting of a flavivirus and hepatitis C virus (HCV).

38. The method of claim 35, wherein the flavivirus is selected from the group consisting of West Nile virus (WNV), Japanese encephalitis virus (JEV), yellow fever virus (YFV), and Dengue fever virus (DEN).

39. The method of claim 35, wherein the Flaviviridae virus is hepatitis C virus (HCV).

40. The method of claim 35, wherein the inhibitor comprises a src family kinase-specific antisense oligonucleotide.

41. The method of claim 40, wherein the antisense oligonucleotide is a phosphorodiamidate morpholino oligomer (PMO).

42. The method of claim 35, wherein the inhibitor comprises src family kinase-specific siRNA.

43. The method of claim 35, wherein the inhibitor comprises a small molecule inhibitor of a src family kinase, wherein the small molecule inhibitor is a molecule, or a pharmaceutically acceptable salt thereof, selected from the Formula group consisting of Formula I, Formula I(b), Formula II, Formula III, Formula IV and Formula V, all according to claim 8.

44. The method of claim 35, wherein the cells suitable to support flavivirus infection are selected from the group consisting of primary human hepatocellular carcinoma derived cells or cell-lines derived therefrom, Huh 7 cells, neuroblastoma cells or cell-lines derived therefrom, SKN-MC cells, and combinations thereof.

45. The method of claim 35, wherein infection precedes contacting of the cells with the agent.

46. The method of claim 35, wherein infection is subsequent to contacting of the cells with the agent.

47-79. (canceled)

80. The method of claim 35, wherein the method identifies an agent having therapeutic utility for the treatment of an human immunodeficiency virus infection, and wherein the cells suitable to support human immunodeficiency virus (HIV) infection are selected from the group consisting of myeloid cells, or T-cells, and combinations thereof.

81. The method of claim 80, wherein the cells are of the myeloid cell line THP-1.

82. The method of claim 80, wherein the cells are of the T-cell leukemia cell line MT-2.

83. The method of claim 80, wherein infection precedes contacting of the cells with the agent.

84. The method of claim 80, wherein infection is subsequent to contacting of the cells with the agent.

85. A method for the treatment of a human immunodeficiency virus (HIV) infection or related condition, comprising administration, to a subject in need thereof, of a therapeutically effective amount of an inhibitor of a human immunodeficiency virus-induced cellular gene sequence selected from the group consisting of HMG20B, HRH1, NP, c-YES, corresponding to SEQ ID NOS:1-9, and combinations thereof, whereby the human immunodeficiency virus (HIV) infection or related condition is diminished, at least to some extent, relative to a non-treated subject.

86. The method of claim 85, wherein the human immunodeficiency virus-induced cellular gene sequence is that of HMG20, corresponding to SEQ ID NOS:1-2.

87. The method of claim 85, wherein the human immunodeficiency virus-induced cellular gene sequence is that of HRH1, corresponding to SEQ ID NOS:3-5.

88. The method of claim 85, wherein the human immunodeficiency virus-induced cellular gene sequence is that of NP, corresponding to SEQ ID NOS:6-7.

89. The method of claim 85, wherein the human immunodeficiency virus-induced cellular gene sequence is that of c-YES, corresponding to SEQ ID NOS:8-9.

90. The method of claim 85, wherein the inhibitor comprises a antisense oligonucleotide specific for the respective human immunodeficiency virus-induced cellular gene sequence.

91. The method of claim 90, wherein the antisense oligonucleotide is a phosphorodiamidate morpholino oligomer (PMO).

92. The method of claim 85, wherein the inhibitor comprises siRNA specific for the respective human immunodeficiency virus-induced cellular gene sequence.

93. The method of claim 85, wherein the inhibitor comprises a small molecule inhibitor specific for the respective human immunodeficiency virus-induced cellular gene sequence.

94. A pharmaceutical composition having utility for the treatment of a human immunodeficiency virus (HIV) infection, comprising, along with a pharmaceutically acceptable carrier or excipient, an inhibitor of a human immunodeficiency virus-induced cellular gene sequence selected from the group consisting of HMG20B, HRH1, NP, c-YES, corresponding to SEQ ID NOS:1-9, and combinations thereof, wherein the inhibitor comprises an agent selected from the group consisting of: a antisense oligonucleotide specific for the respective human immunodeficiency virus-induced cellular gene sequence; siRNA specific for the respective human immunodeficiency virus-induced cellular gene sequence; and a small molecule inhibitor specific for the respective human immunodeficiency virus-induced cellular gene sequence.

95. The composition of claim 94, wherein the human immunodeficiency virus-induced cellular gene sequence is that of HMG20, corresponding to SEQ ID NOS:1-2.

96. The composition of claim 94, wherein the human immunodeficiency virus-induced cellular gene sequence is that of HRH1, corresponding to SEQ ID NOS:3-5.

97. The composition of claim 94, wherein the human immunodeficiency virus-induced cellular gene sequence is that of NP, corresponding to SEQ ID NOS:6-7.

98. The composition of claim 94, wherein the human immunodeficiency virus-induced cellular gene sequence is that of c-YES, corresponding to SEQ ID NOS:8-9.

99. The composition of claim 94, wherein the inhibitor comprises a antisense oligonucleotide specific for the respective human immunodeficiency virus-induced cellular gene sequence.

100. The composition of claim 99, wherein the antisense oligonucleotide is a phosphorodiamidate morpholino oligomer (PMO).

101. The composition of claim 94, wherein the inhibitor comprises siRNA specific for the respective human immunodeficiency virus-induced cellular gene sequence.

102. The composition of claim 94, wherein the inhibitor comprises a small molecule inhibitor specific for the respective human immunodeficiency virus-induced cellular gene sequence.

103. A method for identification of an agent having therapeutic utility for the treatment of a human immunodeficiency virus (HIV) infection or related condition, comprising:
obtaining cells suitable to support a human immunodeficiency virus (HIV) infection;
infecting the cells with the human immunodeficiency virus (HIV);
contacting the infected cells with an agent that inhibits a human immunodeficiency virus-induced cellular gene sequence selected from the group consisting of HMG20B, HRH1, NP, c-YES, corresponding to SEQ ID NOS:1-9, and combinations thereof; and
determining whether the human immunodeficiency virus (HIV) infection is diminished relative to control infected cells not contacted by the agent, whereby the therapeutic agent is identified.

104. The method of claim 103, wherein the human immunodeficiency virus-induced cellular gene sequence is that of HMG20, corresponding to SEQ ID NOS:1-2.

105. The method of claim 103, wherein the human immunodeficiency virus-induced cellular gene sequence is that of HRH1, corresponding to SEQ ID NOS:3-5.

106. The method of claim 103, wherein the human immunodeficiency virus-induced cellular gene sequence is that of NP, corresponding to SEQ ID NOS:6-7.

107. The method of claim 103, wherein the human immunodeficiency virus-induced cellular gene sequence is that of c-YES, corresponding to SEQ ID NOS:8-9.

108. The method of claim 103, wherein the inhibitor comprises a antisense oligonucleotide specific for the respective human immunodeficiency virus-induced cellular gene sequence.

109. The method of claim 108, wherein the antisense oligonucleotide is a phosphorodiamidate morpholino oligomer (PMO).

110. The method of claim 103, wherein the inhibitor comprises siRNA specific for the respective human immunodeficiency virus-induced cellular gene sequence.

111. The method of claim 103, wherein the inhibitor comprises a small molecule inhibitor specific for the respective human immunodeficiency virus-induced cellular gene sequence.

112. The method of claim 103, wherein the cells suitable to support human immunodeficiency virus (HIV) infection are selected from the group consisting of myeloid cells, or T-cells, and combinations thereof.

113. The method of claim 103, wherein the cells are of the myeloid cell line THP-1.

114. The method of claim 103, wherein the cells are of the T-cell leukemia cell line MT-2.

115. The method of claim 103, wherein infection precedes contacting of the cells with the agent.

116. The method of claim 103, wherein infection is subsequent to contacting of the cells with the agent.
